(19) United States
(12) Patent Application Publication
(10) Pub. No.: US 2024/0317830 A1
(43) Pub. Date: Sep. 26, 2024

(54) MULTI-DOMAIN PROTEIN VACCINE

(71) Applicants: Edward Fritsch, Concord, MA (US); Dominik Barthelme, Belmont, MA (US); Cody White, Nashua, NH (US); Lingling Zhang, West Roxbury, MA (US)

(72) Inventors: Edward Fritsch, Concord, MA (US); Dominik Barthelme, Belmont, MA (US); Cody White, Nashua, NH (US); Lingling Zhang, West Roxbury, MA (US)

(21) Appl. No.: 17/767,791

(22) PCT Filed: Oct. 8, 2020
(86) PCT No.: PCT/US20/54785

§ 371 Date: Apr. 8, 2022

Related U.S. Application Data
(60) Provisional application No. 62/912,903, filed on Oct. 9, 2019.

(51) Int. Cl. C07K 14/47; A61K 39/00; C07K 14/78; C07K 14/81

(52) U.S. Cl. CPC C07K 14/4748; C07K 14/78; C07K 14/8139; A61K 39/00; C07K 2319/00

(57) ABSTRACT

Disclosed herein is a protein fusion technology that allows the combination of one or more cancer vaccine epitopes with scaffold domains. Also disclosed herein are polypeptide and polynucleic acid compositions encompassed by the protein fusion technology and the methods of using the same.

### CROSS-REFERENCE TO RELATED APPLICATION

This application claims the benefit of priority to U.S. Provisional Application Ser. No. 62/912,903, filed on 9 Oct. 2019; the entire contents of said application is incorporated herein in its entirety by this reference.

### BACKGROUND

Cancer immunotherapy is directed towards utilizing the patient's immune system to treat cancer. It exploits the fact that cancer cells often have molecules on their surface that can be detected by the immune system, known as tumor antigens, which are often proteins or other macromolecules (e.g. carbohydrates). Active immunotherapy directs the immune system to attack tumor cells by targeting tumor antigens. Passive immunotherapies enhance existing anti-tumor responses and include the use of monoclonal antibodies, lymphocytes and cytokines. Active immunotherapy is directed at inducing new immune responses in the patient. Tumor vaccines are typically composed of tumor antigens and immunostimulatory molecules (e.g., adjuvants, cytokines or TLR ligands) that work together to induce antigen-specific cytotoxic T cells (CTLs) that recognize and lyse tumor cells. Some of the critical barriers to developing curative and tumor-specific immunotherapy is the identification and selection of highly specific and restricted tumor antigens efficiently and delivering the antigens to the subject in need thereof in a manner so as to maximize T cell activation of the subject for the generation of a high anti-tumor immunogenicity.

Tumor neoantigens, which arise as a result of genetic change (e.g., inversions, translocations, deletions, missense mutations, splice site mutations, etc.) within malignant cells, represent the most tumor-specific class of antigens and can be patient-specific or shared. Tumor neoantigens are unique to the tumor cell as the mutation and its corresponding protein are present only in the tumor. They also avoid central tolerance and are therefore more likely to be immunogenic. Therefore, tumor neoantigens provide an excellent target for immune recognition including by both humoral and cellular immunity. However, tumor neoantigens have rarely been used in cancer vaccine or immunogenic compositions due to technical difficulties in identifying them, selecting optimized antigens, and producing neoantigens for use in a vaccine or immunogenic composition. Accordingly, there is still a need for developing additional cancer therapeutics.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SUMMARY

In some aspects, provided herein is a composition comprising a nucleic acid molecule encoding a fusion polypeptide, said fusion polypeptide comprising a polypeptide sequence comprising: (a) one or more antigen polypeptides sequences; and (b) two or more scaffold polypeptides sequences, wherein each of the two or more scaffold polypeptides sequences comprises a human polypeptide sequence, a fragment thereof or a variant thereof, and wherein (i) a molecular mass of the two or more scaffold polypeptide sequences is more than 11 kDa or (ii) each of the two or more scaffold polypeptides sequences comprises at least 21 amino acid residues.

In some aspects, provided herein is a composition comprising a fusion polypeptide, said fusion polypeptide comprising a polypeptide sequence comprising: (a) one or more antigen polypeptides sequences; and (b) one or more scaffold polypeptides sequences, wherein the scaffold polypeptides are selected from Stefin A, Titin-127, a fragment thereof, and a variant thereof.

In some embodiments, at least one of the scaffold polypeptides sequences is connected to at least one of the one or more antigen polypeptides sequences through one or more linker sequences. In some embodiments, the fusion polypeptide is configured to facilitate a cleavage of the linker, a cleavage of at least one of the one or more antigen polypeptides, or presentation of at least one of the one or more antigen polypeptides.

In some aspects, provided herein is a composition comprising a fusion polypeptide or a nucleic acid molecule encoding a fusion polypeptide, said fusion polypeptide comprising a polypeptide sequence comprising: (a) one or more antigen polypeptides sequences, and (b) one or more scaffold polypeptides sequences, wherein at least one of the scaffold polypeptides sequences is connected to at least one of the one or more antigen polypeptides sequences through one or more linker sequences, and wherein the fusion polypeptide is configured to facilitate a cleavage of the linker, a cleavage of at least one of the one or more antigen polypeptides, or presentation of at least one of the one or more antigen polypeptides. In some embodiments, the antigen polypeptides are cancer antigens. In some embodiments, each of the scaffold polypeptides sequences comprises a human polypeptide sequence, a fragment thereof, or a variant thereof.

In some embodiments, the scaffold polypeptides comprise recombinant human polypeptides.

In some embodiments, the scaffold polypeptides are not configured to have targeted binding characteristics.

In some embodiments, the scaffold polypeptides are each independently selected from Titin I27, ubiquitin, Stefin A, 10FN-III, Ig-L filamin A, tenascin, a fragment thereof, and a variant thereof.

In some embodiments, the scaffold polypeptides are each independently selected from Stefin A, Titin I27, a fragment thereof, and a variant thereof.

In some embodiments, the scaffold polypeptides lack (i) post-translational modifications, (ii) intra-peptide disulfide bonds, or both.

In some embodiments, the scaffold polypeptides are non-immunogenic.

In some embodiments, the scaffold polypeptides are not configured to have enzymatic activity.

In some embodiments, each of the scaffold polypeptides is Stefin A, a fragment thereof, or a variant thereof.

In some embodiments, the scaffold polypeptides sequences are the same or different.

In some embodiments, the fusion polypeptide comprises at least 2, 3, 4, 5, 6, 7, 8, 9, 10, 11, 12, 13, 14, or 15 scaffold polypeptides.

In some embodiments, the fusion polypeptide comprises at most 5, 6, 7, 8, 9, 10, 11, 12, 13, 14, 15, or 20 scaffold polypeptides.

In some embodiments, the fusion polypeptide comprises 6 scaffold polypeptides.

In some embodiments, a molecular mass of the scaffold polypeptides sequences is at least 11 kDa, 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, or 200 kDa.

In some embodiments, a molecular mass of the scaffold polypeptides sequences is at most 15 kDa, 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, 200 kDa, or 500 kDa.

In some embodiments, a molecular mass of each of the scaffold polypeptides sequences is at least 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 30 kDa, 40 kDa, 50 kDa, 60 kDa, 70 kDa, or 100 kDa.

In some embodiments, a molecular mass of each of the scaffold polypeptides sequences is at most 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 30 kDa, 40 kDa, 50 kDa, 60 kDa, 70 kDa, or 250 kDa.

In some embodiments, each of the scaffold polypeptides sequences comprises at least 21, at least 30, at least 35, at least 40, at least 45, at least 50, at least 55, at least 60, at least 65, at least 70, at least 75, at least 80, at least 85, at least 90, at least 100, at least 125, at least 150, at least 175, or at least 200 amino acid residues.

In some embodiments, each of the scaffold polypeptides sequences comprises at most 30, at most 35, at most 40, at most 45, at most 50, at most 55, at most 60, at most 65, at most 70, at most 75, at most 80, at most 85, at most 90, at most 100, at most 125, at most 150, at most 175, or at most 200 amino acid residues.

In some embodiments, a molecular mass of the fusion polypeptide is more than 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, or 200 kDa.

In some embodiments, a molecular mass of the fusion polypeptide is more than 75 kDa.

In some embodiments, a molecular mass of the fusion polypeptide is no more than 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, 200 kDa, or 500 kDa.

In some embodiments, each of the one or more antigen polypeptides sequences comprises less than 100, less than 75, less than 50, or less than 35 amino acid residues.

In some embodiments, each of the one or more antigen polypeptides sequences comprises more than 8, 9, 10, 11, 12, 13, 14, or 15 amino acid residues.

In some embodiments, at least one of the scaffold polypeptides sequences is linked, via its N-terminus, to the remaining sequences of the fusion polypeptide.

In some embodiments, at least one of the scaffold polypeptides sequences is linked, via its C-terminus, to the remaining sequences of the fusion polypeptide.

In some embodiments, at least two of the scaffold polypeptides sequences are uninterrupted by the antigen sequences or linker sequences.

In some embodiments, the fusion polypeptide comprises, in the N-terminus to C-terminus direction, a polypeptide sequence having a structure of Formula (Ia),

Aₙ-Sₘ, or Sₘ-Aₙ Formula (Ia),

wherein each S independently represents a scaffold polypeptide sequence and each A independently represents an antigen polypeptide sequence, and wherein m is an integer equal or greater than 2, and n is an integer equal or greater than 1. In some embodiments, n is an integer selected from 1 to 5, and m is 2.

In some embodiments, the fusion polypeptide comprises, in the N-terminus to C-terminus direction, a polypeptide sequence having a structure of Formula (Ib),

Sₒ-Aₙ-Sₘ Formula (Ib),

wherein, each S independently represents a scaffold polypeptide sequence and each A independently represents an antigen polypeptide sequence, and wherein each of o, n, and m is independently an integer equal or greater than 1. In some embodiments, o is 1 or 2, m is 1 or 2, and n is an integer selected from 1 to 5. In some embodiments, the fusion polypeptide comprises a polypeptide sequence having a structure of S-A-S, A-S-A-S, S-A-S-A, S-A-S-A-S, S-A-S-A-S-A-S, S-A-S-A-S-A-S-A-S, or S-A-S-A-S-A-S-A-S-A-S.

In some embodiments, the fusion polypeptide sequence comprises two or more linker sequences.

In some embodiments, the fusion polypeptide comprises, in the N-terminus to C-terminus direction, a polypeptide sequence having a structure of Formula (IIa),

(L-A-L)ₙ-Sₘ, or Sₘ-(L-A-L)ₙ Formula (IIa),

wherein each S independently represents a scaffold polypeptide sequence, each A independently represents an antigen polypeptide sequence, and each L independently represents a linker sequence or is absent, and wherein m is an integer equal or greater than 2, and n is an integer equal or greater than 1. In some embodiments, n is an integer selected from 1 to 5, and m is 2.

In some embodiments, the fusion polypeptide comprises, in the N-terminus to C-terminus direction, a polypeptide sequence having a structure of Formula (IIb),

Sₒ-(L-A-L)ₙ-Sₘ Formula (IIb),

wherein each S independently represents a scaffold polypeptide sequence, each A independently represents an antigen polypeptide sequence, and each L independently represents a linker sequence or is absent, and wherein each of o, n, and m is independently an integer equal or greater than 1. In some embodiments, o is 1 or 2, m is 1 or 2, and n is an integer selected from 1 to 5. In some embodiments, the fusion polypeptide comprises a polypeptide sequence having a structure of S-L-A-L-S, S-A-L-S-L-A-L-S, S-L-A-L-S-L-A, S-L-A-L-S-L-A-L-S, S-L-A-L-S-L-A-L-S-L-A-L-S, S-L-A-L-S-L-A-L-S-L-A-L-S-L-A-S, or S-L-A-L-S-L-A-L-S-L-A-L-S-L-A-S-L-A-L-S.

In some embodiments, the scaffold polypeptides and the antigen polypeptides are situated in the fusion polypeptide in an alternating fashion.

In some embodiments, the fusion polypeptide is in a linear format.

In some embodiments, the fusion polypeptide comprises at least 2, 3, 4, 5, 6, 7, 8, 9, 10, or 20 antigen polypeptides.

In some embodiments, the fusion polypeptide comprises at most 2, 3, 4, 5, 6, 7, 8, 9, 10, 20, 50, or 100 antigen polypeptides.

In some embodiments, the fusion polypeptide comprises 1, 2, 3, 4, 5, or 6 antigen polypeptides.

In some embodiments, each of the scaffold polypeptides sequences is connected to one or two of the antigen polypeptides sequences through at least one of the linker sequences.

In some embodiments, the fusion polypeptide is configured to facilitate a cleavage of the linker or a cleavage of at least one of the one or more antigen polypeptides.

In some embodiments, at least one of the linker sequences comprises a cleavage site.

In some embodiments, the cleavage site is cleavable by a peptidase or a protease.

In some embodiments, the cleavage site is cleavable by a cellular peptidase or protease.

In some embodiments, the fusion polypeptide is configured to facilitate presentation of at least one of the one or more antigen polypeptides.

In some embodiments, at least one of the linker sequences comprises a lysine residue, an arginine residue, a serine residue, a threonine residue, an asparagine residue, a histidine residue, an alanine residue, a glutamine residue, an aspartic acid residue, a methionine residue, a tyrosine residue, a glycine residue, a proline residue, a glutamic acid residue, a tryptophan residue, a phenylalanine residue, a valine residue, an isoleucine residue, a cysteine, a leucine residue or any combination thereof.

In some embodiments, at least one of the linker sequences comprises a lysine residue, an arginine residue, or an alanine residue directly connected to an N-terminus of at least one of the antigen polypeptides.

In some embodiments, at least one of the linker sequences comprises a serine residue, a lysine residue, an arginine residue, or an alanine residue directly connected to a C-terminus of at least one of the antigen polypeptides.

In some embodiments, the one or more antigen polypeptides is a HLA class I antigen polypeptide and wherein at least one of the linker sequences comprises a lysine residue, an arginine residue, a serine residue, a threonine residue, an asparagine residue, a histidine residue, an alanine residue, a glutamine residue, an aspartic acid residue, a methionine residue, a tyrosine residue, a glycine residue, a proline residue, a glutamic acid residue, a tryptophan residue, a phenylalanine residue, a valine residue, an isoleucine residue, a leucine residue a cysteine residue or any combination thereof.

In some embodiments, the one or more antigen polypeptides is a HLA class I antigen polypeptide and at least one of the linker sequences comprises a lysine residue, an arginine residue, or an alanine residue directly connected to an N-terminus of at least one of the antigen polypeptides. In some embodiments, the one or more antigen polypeptides is a HLA class I antigen polypeptide and at least one of the linker sequences comprises a serine residue, a lysine residue, an arginine residue, or an alanine residue directly connected to a C-terminus of at least one of the antigen polypeptides. In some embodiments, the one or more antigen polypeptides is an HLA class II antigen polypeptide and at least one of the linker sequences comprises an aspartic acid residue, a methionine residue, a leucine residue, a valine residue, an isoleucine residue, a tyrosine residue, a phenylalanine residue, a tryptophan residue, or any combination thereof. In some embodiments, the one or more antigen polypeptides is an HLA class II antigen polypeptide and at least one of the linker sequences comprises an aspartic acid residue, a methionine residue, a leucine residue, a tyrosine residue, or a phenylalanine residue directly connected to an N-terminus of at least one of the antigen polypeptides. In some embodiments, the one or more antigen polypeptides is an HLA class II antigen polypeptide and at least one of the linker sequences comprises a methionine residue, a leucine residue, a valine residue, an isoleucine residue, a tyrosine residue, a phenylalanine residue, or a tryptophan residue directly connected to a C-terminus of at least one of the antigen polypeptides.

In some embodiments, at least one of the linker sequences comprises an aspartic acid residue, a methionine residue, a leucine residue, a valine residue, an isoleucine residue, a tyrosine residue, a phenylalanine residue, a tryptophan residue, or any combination thereof.

In some embodiments, at least one of the linker sequences comprises an aspartic acid residue, a methionine residue, a leucine residue, a tyrosine residue, or a phenylalanine residue directly connected to an N-terminus of at least one of the antigen polypeptides.

In some embodiments, at least one of the linker sequences comprises a methionine residue, a leucine residue, a valine residue, an isoleucine residue, a tyrosine residue, a phenylalanine residue, or a tryptophan residue directly connected to a C-terminus of at least one of the antigen polypeptides.

In some embodiments, each of the linker sequences comprises at least 2, 3, 4, 5, 6, 7, 8, 9, 10, 15, 20, 25, or 50 amino acid residues.

In some embodiments, each of the linker sequences comprises at most 2, 3, 4, 5, 6, 7, 8, 9, 10, 15, 20, 25, 50, or 100 amino acid residues.

In some embodiments, the linkers are flexible.

In some embodiments, the linker sequences are the same or different.

In some embodiments, the solubility of the fusion polypeptide in an aqueous solvent is at least 1.1, 2, 3, 4, 5, 6, 7, 8, 9, 10, 20, 30, 40 or 50 fold higher than the solubility of its corresponding polypeptide lacking the scaffold polypeptides, measured in the same solvent.

In some embodiments, a solubility of the fusion polypeptide in an aqueous formulation is at least 1.1, 2, 3, 4, 5, 6, 7, 8, 9, 10, 20, 30, 40 or 50 fold higher than a solubility of its corresponding polypeptide lacking the scaffold polypeptides in the aqueous formulation.

In some embodiments, a serum solubility of the fusion polypeptide is at least 1.1, 2, 3, 4, 5, 6, 7, 8, 9, or 10 fold higher than a serum solubility of its corresponding polypeptide lacking the scaffold polypeptides.

In some embodiments, a serum half-life of the fusion polypeptide is at least 1.1, 2, 3, 4, 5, 6, 7, 8, 9, or 10 fold longer than a serum half-life of its corresponding polypeptide lacking the scaffold polypeptides. In some embodiments, a serum half-life of the fusion polypeptide is at least 10, 50, 100, 200, 300, 400, 500, 600, 700, 800, 900, 1000, 5000 or 10000 fold higher than a serum solubility of its corresponding polypeptide lacking the scaffold polypeptides.

In some embodiments, an accumulation of the fusion polypeptide in lymph node following administration to a subject is at least 1.1, 2, 3, 4, 5, 6, 7, 8, 9, 10, 20, 30, 40, 50, 75, 100, 150, 200, 250, 500, 1000, 5000 or 10000 fold higher than an accumulation of its corresponding polypeptide lacking the scaffold polypeptides as measured by an average radiant efficiency.

In some embodiments, an antigen-specific T cell response against the fusion polypeptide following administration to a subject is at least 1.1, 2, 3, 4, 5, 6, 7, 8, 9, or 10, 20, 30, 40, 50, 75, 100, 150, 200, 250 or 300 fold higher than an antigen-specific T cell response against its corresponding polypeptide lacking the scaffold polypeptides as measured by a frequency of antigen specific T cells or an increase in secretion of IFNγ.

In some embodiments, the fusion polypeptide comprises one or more binding moieties configured to bind to an antigen presenting cell, to an adjuvant, or to a reagent.

In some embodiments, the multivalent fusion polypeptide is functionalized by mixing with an anti-scaffold antibody or fragment thereof coupled to a functionalizing agent such as a dendritic cell (DC)-targeting domain, an adjuvant or an immune modulator.

In some embodiments, the antigen presenting cell is a dendritic cell (DC), a macrophage, a Langerhans cell, or a B cell.

In some embodiments, the one or more binding moieties are configured to bind to one or more receptors expressed on a dendritic cell.

In some embodiments, the one or more receptors comprise a C-type lectin receptor, a scavenger receptor, an F4/80 Receptor, a DC-specific transmembrane protein (e.g., DC-STAMP), an Fc receptor, or any combination thereof.

In some embodiments, the one or more receptors comprise Clec9a or XCR1.

In some embodiments, at least one of the binding moieties is comprised in the scaffold polypeptides.

In some embodiments, at least one of the binding moieties is directly connected to an N-terminus or a C-terminus of one of the scaffold polypeptides.

In some embodiments, at least one of the binding moieties is directly connected to an N-terminus or a C-terminus of one of the antigen polypeptides,

In some embodiments, at least one of the binding moieties is connected to a linker polypeptide.

In some embodiments, in the N-terminus to C-terminus direction, at least one of the binding moieties is terminally linked to a first or a last scaffold polypeptide of the scaffold polypeptides. In some embodiments, the composition comprises one or more binding moieties capable of binding to the fusion polypeptide. In some embodiments, the one or more binding moieties bind to the fusion polypeptide. In some embodiments, the one or more binding moieties are conjugated to the fusion polypeptide.

In some embodiments, the one or more cancer antigen polypeptides comprise a plurality of antigen polypeptides. In some embodiments the one or more cancer antigen polypeptides are auto-immune peptides.

In some embodiments, the antigen polypeptides are neoantigen peptides.

In some embodiments, the neoepitope of each peptide is unique. In some embodiments, each of the cancer neoantigen peptides or a portion thereof binds to a protein encoded by an HLA allele expressed by the subject and is encoded by at least one expressed gene of the subject's cancer cells, and wherein at least one of the cancer neoantigen peptides or a portion thereof comprises one or more mutations that are not present in a normal tissue of the subject.

In some embodiments, at least one of the one or more mutations is: (A) a point mutation and the cancer neoantigen peptide binds to the protein encoded by an HLA allele expressed by the subject with an IC50 less than 500 nM and a greater affinity than a corresponding wild-type peptide, (B) a splice-site mutation, (C) a frameshift mutation, (D) a read-through mutation, or (E) a gene-fusion mutation.

In some embodiments, a first cancer neoantigen peptide of the plurality binds to a protein encoded by a first HLA allele expressed by the subject and a second cancer neoantigen peptide of the plurality binds to a protein encoded by a second HLA allele expressed by the subject, wherein the first and second HLA alleles expressed by the subject are different HLA alleles.

In some embodiments, at least one of the cancer neoantigen peptides binds to the protein encoded by the HLA allele expressed by the subject with an IC50 of less than 250 nM.

In some embodiments, the fusion polypeptide is expressed in a host cell. In some embodiments, the fusion polypeptide is a synthetic construct.

In some embodiments, the nucleic acid molecule encodes at least 2, 3, 4, 5, 6, 7, 8, 9, 10, or 20 different fusion polypeptides.

In some embodiments, the nucleic acid molecule encodes at most 2, 3, 4, 5, 6, 7, 8, 9, 10, or 20 different fusion polypeptides.

In some embodiments, the nucleic acid molecule is RNA or DNA.

In one aspect, disclosed herein is a fusion polypeptide encoded by the described nucleic acid molecule.

In one aspect, disclosed herein is a composition comprising the described fusion polypeptide. In some embodiments, the composition comprises one or more binding moieties bound to the fusion polypeptide. In some embodiments, the one or more binding moieties are conjugated to the fusion polypeptide.

In one aspect, disclosed herein is a nucleic acid molecule encoding: two or more scaffold polypeptides interspaced by one or more linkers; and one or more restriction sites located on at least one of the linkers, wherein (i) a molecular mass of the two or more scaffold polypeptide sequences is more than 11 kDa or (ii) each of the two or more scaffold polypeptides sequences comprises at least 21 amino acid residues. In some embodiments, the scaffold polypeptide is encoded by RNA or DNA.

In one aspect, disclosed herein is a plurality of nucleic acid molecules comprising: a first nucleic acid molecule comprising, a nucleic acid sequence encoding a first antigen polypeptide, and a nucleic acid sequence encoding a first scaffold polypeptide; and a second nucleic acid molecule comprising, a nucleic acid sequence comprising a sequence complementary to the nucleic acid sequence encoding a first antigen polypeptide, and a nucleic acid sequence encoding a second scaffold polypeptide.

In another aspect, disclosed herein is a plurality of nucleic acid molecules comprising: a first nucleic acid molecule comprising, a nucleic acid sequence encoding a first antigen polypeptide, a nucleic acid sequence encoding a first scaffold polypeptide, and a nucleic acid sequence encoding a second antigen polypeptide; and a second nucleic acid molecule comprising, a nucleic acid sequence comprising a sequence complementary to the nucleic acid sequence encoding a second antigen polypeptide, and a nucleic acid sequence encoding a second scaffold polypeptide.

In yet another aspect, disclosed herein is a plurality of nucleic acid molecules comprising: a first nucleic acid molecule comprising, a nucleic acid sequence encoding a first antigen polypeptide, a nucleic acid sequence encoding a first scaffold polypeptide, and a nucleic acid sequence encoding a second antigen polypeptide; and a second nucleic acid molecule comprising a nucleic acid sequence comprising a sequence complementary to the nucleic acid sequence encoding a second antigen polypeptide, a nucleic acid sequence encoding a second scaffold polypeptide, and a nucleic acid sequence encoding a third antigen polypeptide.

In some embodiments, the first and the second scaffold sequences are the same.

In some embodiments, the first antigen polypeptide and the second antigen polypeptides are different.

In some embodiments, the first, the second, and the third antigen polypeptides are different.

In some embodiments, the nucleic acid molecules are RNA or DNA.

In one aspect, disclosed herein is a pharmaceutical composition comprising: a pharmaceutically acceptable excipient, carrier, or diluent, and the described composition or the described fusion polypeptide.

In some embodiments, the pharmaceutical composition comprises an adjuvant.

In some embodiments, the adjuvant is polyIC:LC.

In some embodiments, the pharmaceutical composition comprises a pH modifier.

In some embodiments, the pharmaceutical composition comprises a second therapeutic agent. In some embodiments, the second therapeutic agent is an immunomodulator, a cytokine or chemokine or a checkpoint inhibitor. In some embodiments the second therapeutic is administered to a subject in need thereof, prior to administering the pharmaceutical composition comprising the fusion polypeptide or the nucleic acid encoding the fusion polypeptide. In some embodiments the second therapeutic is administered concomitantly with administering the pharmaceutical composition comprising the fusion polypeptide or the nucleic acid encoding the fusion polypeptide. In some embodiments the second therapeutic is administered after administering the pharmaceutical composition comprising the fusion polypeptide or the nucleic acid encoding the fusion polypeptide.

In one aspect, disclosed herein is a method of producing an immunogenic fusion polypeptide, the method comprising, expressing the described nucleic acid molecule in a genetically modified cell, thereby producing the immunogenic fusion polypeptide.

In another aspect, disclosed herein is a method of producing an immunogenic fusion polypeptide, the method comprising: providing a nucleic acid molecule as described herein; inserting one or more nucleic acid molecules encoding one or more antigen polypeptides to at least one of the restriction sites, thereby producing a new nucleic acid molecule; and expressing the new nucleic acid molecule in a genetically modified cell, thereby producing the immunogenic fusion polypeptide.

In some embodiments, the one or more nucleic acid molecules encoding one or more antigen polypeptides are inserted through an isothermal reaction or by restriction enzyme based cloning.

In one aspect, disclosed herein is a method of producing an immunogenic fusion polypeptide, the method comprising: providing a plurality of nucleic acid molecules as described herein; joining the nucleic acid molecules through hybridization; and expressing the joined nucleic acid molecules in a genetically modified cell, thereby producing the immunogenic fusion polypeptide.

In some embodiments, the fusion polypeptide is expressed in a bacterial expression system.

In some embodiments, the bacterial expression system is an Escherichia coli expression system.

In one aspect, provided herein is a method of producing an immunogenic fusion polypeptide, the method comprising, (a) providing a nucleic acid molecule described in any of the paragraphs above, or specifically paragraphs 89-96, (b) inserting one or more nucleic acid molecules encoding one or more antigen polypeptides to at least one of the restriction sites, thereby producing a new nucleic acid molecule, and (c) expressing the new nucleic acid molecule by in vitro translation or in a genetically modified cell, thereby producing the immunogenic fusion polypeptide.

In one aspect, provided herein is a method of producing an immunogenic fusion polypeptide, the method comprising, (a) providing a plurality of nucleic acid molecules as described in any one of the paragraphs described above or specifically paragraphs 89-96, (b) joining the nucleic acid molecules through hybridization; and (c) expressing the joined nucleic acid molecules by in vitro translation or in a genetically modified cell, thereby producing the immunogenic fusion polypeptide. In some embodiments the fusion polypeptide is expressed in a bacterial expression system. In some embodiment, the bacterial expression system is an E. coli expression system.

In some embodiments, the fusion polypeptide is expressed by in vitro translation. In some embodiments, the method further comprises a gap-filling step and/or a ligation step. In some embodiments the gap-filling step comprises a polymerase-mediated gap-filling step.

In one aspect, disclosed herein is a method of treating or preventing cancer in a human subject in need thereof comprising, administering to the subject in need thereof the pharmaceutical composition as described herein.

In some embodiments, the pharmaceutical composition comprises a plurality of neoantigen peptides.

In some embodiments, the pharmaceutical composition comprises a plurality of fusion polypeptides.

In some embodiments, the pharmaceutical composition is administered intravenously or subcutaneously.

In some embodiments, a dose of the fusion polypeptide is divided into at least 2, at least 3, at least 4 or at least 5 sub-doses.

In some embodiments, each sub-dose of the fusion polypeptide comprises 1, 2, 3, 4, 5, or more fusion polypeptides.

In some embodiments, each fusion polypeptide is administered at a dose of from 0.01-100 μg.

In some embodiments, each fusion polypeptide is administered at a dose of from 100 μg-10 mg.

In some embodiments, a total dose of the fusion polypeptides administered is from 0.1-100 mg.

In some embodiments, the cancer is a solid tumor.

In some embodiments, the cancer is melanoma, lung cancer, or bladder cancer.

In one aspect, provided herein is a library comprising a plurality of recombinant expression constructs, wherein each expression construct of the plurality comprises: (a) a promoter sequence; (b) a sequence encoding a fusion polypeptide comprising: (i) a start codon downstream of the promoter sequence; (ii) a first polynucleotide sequence downstream of the start codon, wherein the first polynucleotide sequence comprises a distinct template polynucleotide sequence, wherein the distinct template from polynucleotide sequence (A) is derived from a sample comprising diseased cells from a subject with a disease and (B) encodes a peptide sequence of a protein encoded by the diseased cells from the subject; and (iii) a second polynucleotide sequence downstream of the first polynucleotide sequence, wherein the second polynucleotide sequence comprises (A) a frame check sequence and a sequence encoding one or more affinity tags downstream of the frame check sequence, or (B) a frame check sequence comprising a sequence encoding an affinity tag. In some embodiments, the frame check sequence operates to terminate translation of the fusion polypeptide when the distinct template polynucleotide sequence or copy thereof is out of frame with the sequence encoding an affinity tag. In some embodiments, the frame check sequence has a formula of:

N₁-N₂-N₃-N₄-N₅-N₆-N₇-N₈-N₉:

wherein each N is independently a nucleic acid selected from the group consisting of A, T, U, C and G; each of N₁-N₂-N₃ and N₄-N₅-N₆ and N₇-N₈-N₉ is not a stop codon; and each of N₂-N₃-N₄ and N₆-N₇-N₈ is a stop codon. In some embodiments, the frame check sequence encodes Val-Gly-Ser. In some embodiments, the frame check sequence encodes a linker that links the first polynucleotide sequence to the sequence encoding the affinity tag.

In some embodiments, the sequence encoding an affinity tag is a frame check sequence. In some embodiments, the affinity tag is a fragment crystallizable region (Fc region) or peptide sequence that binds to an Fc receptor, a GST-tag, a His-tag, a peptide sequence that binds to Protein A, a peptide sequence that binds to Protein G, or an epitope of an antibody or binding fragment thereof. In some embodiments, the affinity tag is non-immunogenic. In some embodiments, the affinity tag is human. In some embodiments, the sequence encoding the affinity tag encodes a size-enhancing polypeptide; and/or (ii) each expression construct of the library comprises a sequence encoding a size enhancing polypeptide. In some embodiments, the sequence encoding the size enhancing polypeptide is downstream of the first polynucleotide sequence and/or at least one of the sequences encoding the size enhancing polypeptide is upstream of the distinct template polynucleotide sequence. In some embodiments, the affinity tag is an epitope of a size enhancing polypeptide. In some embodiments, the sequence encoding the size enhancing polypeptide encodes a plurality of at least 2, 3, 4, 5, 6, 7, 8, 9 or 10 or more size enhancing polypeptides. In some embodiments, at least 2, 3, 4, 5, 6, 7, 8, 9, 10 or more size enhancing polypeptides of the plurality of size enhancing polypeptides are the same. In some embodiments, the sequence encoding the size enhancing polypeptides encodes one or more linkers between two or more of the size enhancing polypeptides. In some embodiments, the molecular weight of the plurality of size enhancing polypeptides is at least 15 kDa. In some embodiments, the molecular weight of the plurality of size enhancing polypeptides is no more than 200 kDa. In some embodiments, the molecular weight of the plurality of size enhancing polypeptides is from 40 kDa to 80 kDa or from 50 kDa to 70 kDa.

In some embodiments, the distinct template polynucleotide sequence encodes a peptide sequence of a protein expressed by the disease cells from the subject, wherein the subject is human; wherein the disease is cancer. In some embodiments, the cancer is a melanoma, a bladder cancer or a lung cancer.

In some embodiments, the disease is an autoimmune disease.

In some embodiments, the sample comprises a biopsy, a blood sample or a peripheral blood mononuclear cell (PBMC) sample and wherein the sample comprises cells with the disease. In some embodiments, each expression construct of the plurality comprises the same promoter, the same start codon, the same frame check sequence, the same sequence encoding the size enhancing polypeptide or any combination thereof. In some embodiments, each of the distinct template polynucleotides sequences is at least 24 bps in length, at least 45 bps in length, at most 450 bps in length, at most 250 bps in length, from 24-300 bps in length, from 45-450 bps in length or any combination thereof.

In some embodiments, each of the distinct template polynucleotides sequences is cDNA.

In some embodiments, each of the distinct template polynucleotides sequences is derived from an RNA molecule.

In some embodiments, each of the distinct template polynucleotides sequences is derived from genomic DNA (gDNA). In some embodiments, the library comprises a plurality of distinct template polynucleotides that represent at least 5% up to 100% of an exome.

In some embodiments, wherein the library comprises a plurality of distinct template polynucleotides that encode at least 5% up to 100% of the peptide sequences of a proteome. In some embodiments, the library comprises a plurality of distinct template polynucleotides that encode peptide sequences derived from at least 5% of the proteins of a proteome. In some embodiments, the exome or proteome is the exome or proteome of the diseased cells. In some embodiments, about 5% to about 25% of polypeptides expressed from library comprise the affinity tag. In some embodiments, about 20% to about 40% of polypeptides expressed from library comprise the affinity tag. In some embodiments, about 50% to about 90% of polypeptides expressed from the library that comprise at least 60 amino acid residues do not comprise the affinity tag. In some embodiments, about 85% to about 95% of the polypeptides expressed from the in frame expression constructs comprise a polypeptide that is attached to the affinity tag. In some embodiments, the library comprises at least 100, 1000, 10000, 100000, 1×10⁶, 1×10⁷, 1×10⁸, 1×10⁹ or up to 1×10¹⁰ distinct template polynucleotide sequences. In some embodiments, the distinct template polynucleotides encode peptide sequences of at least 10, 50, 100, 500, 1000, 10000 or 100000 distinct proteins.

In some embodiments, at least about 10, 100, 500, 1000, 5000 or 10000 of the distinct template polynucleotides encode a mutant peptide sequence or mRNA translation products derived from aberrantly transcribed, spliced or translated mRNA in the diseased cell or from gene rearrangements.

In some embodiments, the mutant peptide sequence is a cancer-cell specific mutant peptide sequence. In some embodiments, the mutant peptide sequence comprises a point mutation or an indel.

In some embodiments, the expression construct further comprises one or more scaffold polypeptide sequences upstream of the one or more antigen polypeptide sequences. In some embodiments, the expression construct further comprises a linker sequence downstream of the one or more scaffold polypeptide sequences and upstream of the one or more antigen polypeptide sequences.

In some embodiments, the first polynucleotide sequence further comprises an upstream adaptor sequence immediately upstream of the distinct template polynucleotide sequence and/or a downstream adaptor sequence immediately downstream of the distinct template polynucleotide sequence.

In some embodiments, the fusion protein is multivalent.

Provided herein is a polypeptide library encoded by any one of the preceding claims.

In some embodiments, the polypeptides of the polypeptide library are expressed in a host cell, are expressed using an in vitro translation system, and/or are expressed from a phage vector. In some embodiments, the phage vector is a filamentous phage vector, such as M13 phage vector, or f1 phage vector, or fd phage vector.

In some embodiments, the polypeptides of the library are expressed as parts of virus-like particles (VLP). In some embodiments, the VLP like particles are expressible in a host cell, such as a bacterial cell, such as an Escherichia coli cell. In some embodiments, the VLP self assembles in the host cell. In some embodiments, the host cell is a bacteria.

In some embodiments, the polypeptides of the library are in vitro translated polypeptides.

In some embodiments, the polypeptide library comprises a plurality of isolated polypeptides.

In some embodiments, the polypeptides of the library are isolated or purified or enriched via at least the affinity tag.

In one aspect, provided herein is a personalized recombinant proteome library comprising a plurality of recombinant fusion polypeptides expressed in a host cell, the plurality of recombinant fusion polypeptides comprising a plurality of polypeptide sequences encoded by a plurality of at least 10 distinct template polynucleotide sequences from a sample comprising diseased cells from a subject with a disease, and wherein each of the polypeptide sequences encoded by the plurality of at least 10 distinct template polynucleotides comprises, in a N to C direction (i) a polypeptide sequence encoded by a distinct template polynucleotide of the at least 10 distinct template polynucleotide sequences, and (ii): (A) a frame check sequence and a sequence encoding an affinity tag downstream of the frame check sequence, or (B) a frame check sequence comprising a sequence encoding an affinity tag, and/or a size enhancing polypeptide sequence that is at least 40 kDa.

In one aspect, provided herein is a personalized recombinant proteome library, comprising a plurality of recombinant fusion polypeptides expressed in a host cell, the plurality of recombinant fusion polypeptides comprising a plurality of polypeptide sequences encoded by a plurality of at least 1000 distinct template polynucleotide sequences from a sample comprising diseased cells from a subject with a disease.

In one aspect, provided herein is a vaccine composition comprising the polypeptide library or personalized recombinant proteome library as described above.

In one aspect, provided herein is a method of treatment comprising administering the polypeptide library or personalized recombinant proteome library as described above to the subject.

In one aspect, provided herein is a pharmaceutical composition comprising the polypeptide library or personalized recombinant proteome library as described above and a pharmaceutically acceptable excipient.

In one aspect, provided herein is a method of treatment, comprising administering the pharmaceutical composition described above to the subject.

In one aspect, provided herein is a population of cells comprising a polypeptide library or a personalized recombinant proteome library described herein. In some embodiments, each cell of the population of cells expresses a single distinct polypeptide sequence encoded by the plurality of distinct template polynucleotides.

Provided herein is a method of constructing an expression vector, comprising: (a) providing a plurality of distinct template polynucleotides from a sample comprising diseased cells from a subject with a disease; (b) attaching adaptor sequences to the plurality of distinct template polynucleotides; thereby forming a plurality of distinct adaptor tagged template polynucleotides; (c) amplifying the distinct adaptor tagged template polynucleotides; (d) inserting the amplified distinct adaptor tagged template polynucleotides into a vector, thereby forming a library of recombinant expression constructs; (e) expressing polypeptides encoded by the library of recombinant expression constructs; and enriching for the expressed polypeptides.

In some embodiments, the method comprises contacting the plurality of distinct polynucleotides with a library of exome capture oligonucleotides, wherein said capture oligonucleotides comprise target sequences.

In some embodiments, the exome capture oligonucleotides are immobilized on a surface.

In some embodiments, amplifying comprises amplifying with random primers.

In some embodiments, amplifying comprises amplifying without bias or is not target specific.

In some embodiments, the method comprises hybridizing the plurality of distinct template polynucleotide sequences to a plurality of reference polynucleotide sequences from a reference sample or from non-diseased cells from the subject with a disease.

In some embodiments, the method further comprises selectively enriching duplexed polynucleotides comprising a mismatch from duplexed polynucleotides that do not comprise a mismatch.

In some embodiments, selectively enriching comprises contacting the duplexed polynucleotides to an agent that specifically binds to the duplexed polynucleotides comprising a mismatch.

In some embodiments, the agent is a DNA base mismatch recognizing agent.

In some embodiments, the agent is a MutS protein or a functional fragment thereof. In some embodiments, the MutS protein is derived from a bacterial species, including but not limited to E coli, Salmonella sp., Haemophilus sp., Azotobacter sp., Acinetobacter sp., Bacillus sp., Borrelia sp., Chlamydia sp., Helicobacter sp., Neisseria sp., Deinococcus radiodurans and Streptococcus sp. among others.

In some embodiments, the mismatch comprises a single nucleotide variant, an insertion or a deletion.

In some embodiments, the method further comprises combining (i) selectively enriched duplexed polynucleotides comprising a mismatch with (ii) a plurality of distinct template polynucleotides from a sample comprising diseased cells from a subject with a disease that have not been selectively enriched.

In some embodiments, the plurality of distinct target polynucleotides comprises sequences derived from a whole exome and/or from exomic-intronic boundary sequences.

In some embodiments, the plurality of distinct target polynucleotides comprises sequences derived from a subset of an exome based on expression data of one or more cancer types.

In some embodiments, the plurality of distinct target polynucleotides comprises sequences derived from a whole genome.

In some embodiments, the plurality of distinct target polynucleotides comprises sequences derived from a set of whole genome sequences enriched with a library of exome capture oligonucleotides.

In some embodiments, the method further comprises cloning the plurality of distinct target polynucleotides into an expression vector.

In some embodiments, the set of reference polynucleotides comprises a reference genome exon capture probe set or polynucleotides from a non-tumor sample from the subject.

In some embodiments, the library of exome capture oligonucleotides comprises high frequency human polymorphism sequences.

In some embodiments, the reference sample comprises a non-tumor sample from the subject.

In some embodiments, the method further comprises performing reverse transcription.

In some embodiments, the method does not further comprise sequencing.

In some embodiments, the method does not further comprise predicting or determining binding of an epitope to a protein encoded by an HLA allele and/or presentation of an epitope by a protein encoded by an HLA allele.

In some embodiments, attaching adaptor sequences to the plurality of distinct template polynucleotides comprises attaching strand specific adapter sequences.

In some embodiments, the distinct target polynucleotide sequences are from gDNA.

In some embodiments, the distinct target polynucleotide sequences are from derived from mRNA or exomic sequences.

In some embodiments, enriching comprises enriching for affinity tag containing expressed polypeptides.

In some embodiments, the method further comprises fragmenting polynucleic acids of the sample.

In some embodiments, the method further comprises shearing genomic DNA of the sample.

In some embodiments, the sample is a FFPE sample.

Provided herein is a method of treatment comprising performing the method described above and administering to the subject, the enriched expressed polypeptides.

### BRIEF DESCRIPTION OF THE DRAWINGS

The features of the present disclosure are set forth with particularity in the appended claims. A better understanding of the features and advantages of the present will be obtained by reference to the following detailed description that sets forth illustrative embodiments, in which the principles of the disclosure are utilized, and the accompanying drawings of which:

FIG. 1 illustrates an option, option A, of the polypeptide configurations.

FIG. 2 illustrates another option, option B, of the polypeptide configurations.

FIG. 3 illustrates yet another option, option C, of the polypeptide configurations.

FIG. 4 illustrates some of the considerations for scaffold domain selection and six exemplary scaffold domains.

FIG. 5A illustrates test results of exemplary scaffold domains in a solubility challenge test and the expression level test as hexameric polybody.

FIG. 5B illustrates solubility challenge test results using the indicated scaffold domains to solubilize the Alg8 peptide.

FIG. 5C illustrates solubility challenge test results using Stefin-A as a scaffold domain to solubilize the indicated peptides. S: soluble fraction; I: insoluble fraction.

FIG. 6 illustrates the polybody constructs that were cloned for the expression level test.

FIGS. 7A-7C illustrate the lymph node accumulation test for polybody constructs having one to six scaffold domains. FIG. 7A shows an image of the banding patterns of polybody constructs stained with Coomassie Blue; FIG. 7B shows the injection and measurement schedule for the accumulation test; and FIG. 7C shows the injection sites in mice.

FIGS. 8A-8D illustrate the lymph node accumulation of fluorophore-labeled polybody constructs in comparison with synthetic long peptides. FIG. 8A shows the accumulation of fluorescence in the inguinal lymph nodes in mice; FIG. 8B shows the accumulation of fluorescence in the axillary lymph nodes in mice; and FIG. 8C shows the intensity of fluorescence accumulation of a hexamer polybody in comparison with synthetic long peptides. FIG. 8D shows FACS analysis of lymph nodes.

FIG. 9 illustrates the fluorophore accumulation of polybody constructs in comparison with synthetic long peptides in the left and right inguinal lymph nodes (iLN) and axillary lymph nodes (aLN) in mice.

FIGS. 10A-10C illustrate the in vivo immunogenicity of polybody constructs vs synthetic long peptides using MC38 tumor model epitopes. FIG. 10A and FIG. 10B show the immune response to the polybody constructs and the synthetic long peptides comprising Reps1 and Adpgk epitope, respectively. FIG. 10C shows the configuration of polybody 1 (PB1), polybody 2 (PB2), and polybody 3 (PB3).

FIGS. 11A-11B illustrate exemplary polybody constructs. FIG. 11A shows an exemplary polybody construct comprising non-functional scaffold domains, and FIG. 11B shows an exemplary polybody construct comprising functional and non-functional scaffold domains.

FIGS. 12A-12B illustrate exemplary polybody constructs. FIG. 12A shows an exemplary polybody construct comprising non-functional scaffold domains, and FIG. 12B shows an exemplary polybody construct comprising a functionalized peptide tag.

FIG. 13 illustrates a manufacturing pipeline of the fusion polypeptide.

FIG. 14 illustrates a graphic representation of a method for preparing an exome capture library of random primed cDNA prepared from RNA extracted from formalin fixed paraffin embedded (FFPE) samples, for generating artificial mini-proteome vaccine (AMPVax).

FIG. 15A illustrates a graphic representation of an AmpVax enriched exome capture method. FIG. 15B illustrates a graphic representation of an AmpVax enriched exome capture method using MutS enrichment. FIG. 15C illustrates a graphic representation of an AmpVax enriched exome capture method using normal genomic DNA for MutS bait. FIG. 15D illustrates a graphic representation of the workflow of an AmpVax enriched exome capture method and an AmpVax enriched exome capture method using MutS enrichment.

FIG. 16A illustrates a graphic representation of an exemplary polybody-fusion construct of an exome capture library. FIG. 16B illustrates a graphic representation of an exemplary polybody-fusion construct of an exome capture library. FIG. 16C illustrates a graphic representation of an exemplary polybody-fusion construct of an exome capture library. FIG. 16D illustrates a graphic representation of an exemplary polybody-fusion construct of an exome capture library. FIG. 16E illustrates a graphic representation of an exemplary polybody-fusion construct of an exome capture library.

FIG. 17 illustrates a graphic representation of an exemplary method of constructing plasmids for expressing polybody-fusions.

FIG. 18A illustrates a graphical representation of distribution/evenness following 50× enrichment with ⅕ SNPs and reference exome MutS capture. FIG. 18B illustrates a graphical representation of distribution/evenness following 50× enrichment with ⅕ SNPs and normal DNA MutS capture. FIG. 18C illustrates a graphical comparison of a reference exome without SNP design vs. normal DNA for MutS capture.

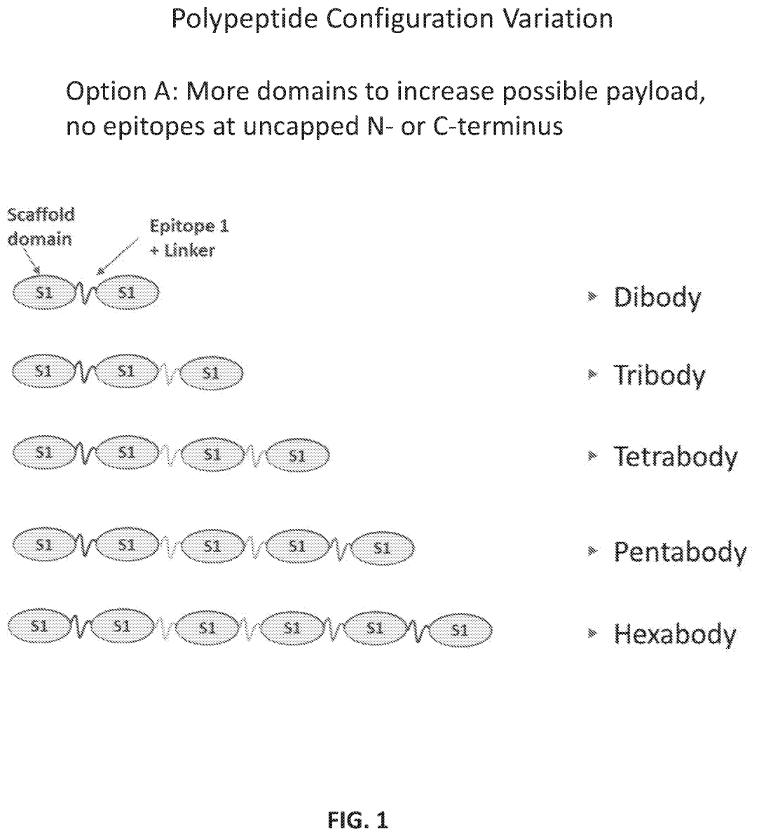

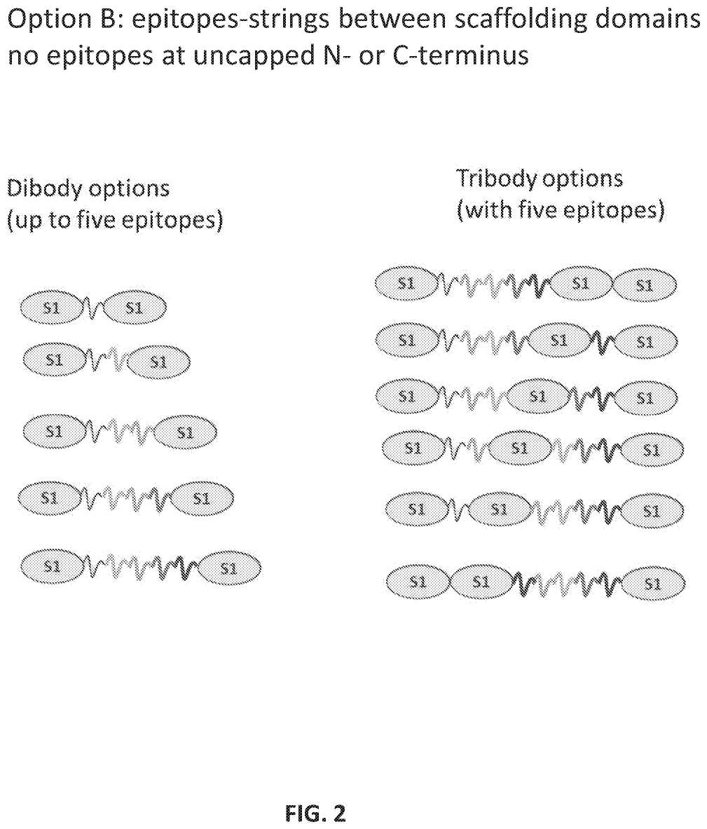

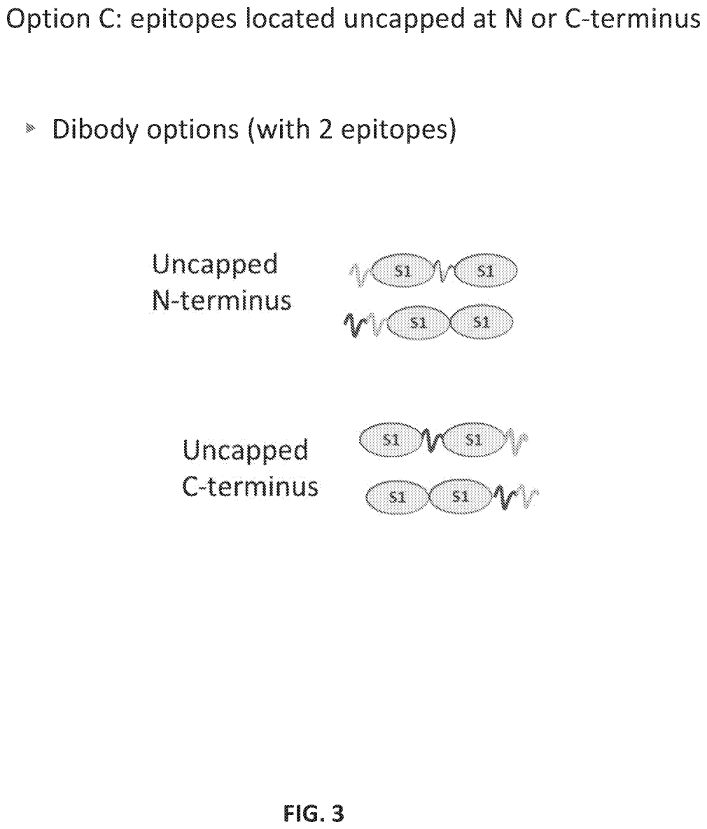

### DETAILED DESCRIPTION

The present disclosure focuses on an important aspect of therapeutic development comprising peptides or nucleic acids encoding therapeutic peptides, which encompasses modification of the peptides for stability and targeted delivery within the recipient of the therapeutic. In some aspects the peptides are modified for increased immunogenicity. In some aspects the peptides are neoantigen peptides. In some aspects the peptides are neoantigens for treating a disease in a subject, wherein the disease is an immune disease. In some aspects the disease is cancer. In some aspects the modifications include peptide fusions. Provided herein are methods and pharmaceutical compositions relating to the delivery of neoantigens in the fused form. Additionally, neoantigen peptides can be delivered efficiently to lymph nodes in the fused form, by which a larger number of naïve T lymphocyte populations can be exposed and primed against the neoantigen peptides.

In one aspect, described herein is a highly modular protein-fusion technology, named polybodies, which allows efficient delivery, tissue uptake and functionality of the neoantigen peptides. In some embodiments, the polybodies protect the neoantigen peptides from enzymatic digestion in serum following subcutaneous injection. In some embodiments, the polybodies help target neoantigen peptides to the lymph node. In some embodiments, the polybodies help lymph node retention of neoantigen peptides. In some embodiments, the polybody help in the effective priming and activation of T lymphocytes.

In some embodiments, modular protein fusion technology allows the combination of multiple cancer vaccine epitopes (e.g. neoantigens, tumor associated antigens) on a single protein construct. In some embodiments, the epitopes are surrounded by scaffold domains. In some embodiments, these scaffold domains keep the antigenic peptides more soluble and prevent them from premature degradation caused by circulating proteases and peptidases in patients. In some embodiments, these scaffolding domains are functionalized to add desired functions to polybodies to further increase the immunogenicity and anti-tumor efficacy of the vaccine.

Described herein are new immunotherapeutic agents and uses thereof based on the discovery of neoantigens arising from mutational events unique to an individual's tumor. Accordingly, the present disclosure described herein provides peptides, polynucleotides encoding the peptides, and peptide binding agents that can be used, for example, to stimulate an immune response to a tumor associated antigen or neoepitope, to create an immunogenic composition or cancer vaccine for use in treating disease.

The following description and examples illustrate embodiments of the present disclosure in detail. It is to be understood that this present disclosure is not limited to the particular embodiments described herein and as such can vary. Those of skill in the art will recognize that there are numerous variations and modifications of this present disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The section headings used herein are for organizational purposes only and are not to be construed as limiting the subject matter described.

Although various features of the present disclosure may be described in the context of a single embodiment, the features may also be provided separately or in any suitable combination. Conversely, although the present disclosure may be described herein in the context of separate embodiments for clarity, the present disclosure may also be implemented in a single embodiment.

The various terminology used herein is for the purpose of describing particular cases only and is not intended to be limiting. In this application, the use of the singular includes the plural unless specifically stated otherwise. As used herein, the singular forms “a”, “an” and “the” are intended to include the plural forms as well, unless the context clearly indicates otherwise. As is understood herein, “or” means “and/or” unless stated otherwise. The terms “and/or” and “any combination thereof” and their grammatical equivalents as used herein, can be used interchangeably. These terms can convey that any combination is specifically contemplated. Solely for illustrative purposes, the following phrases “A, B, and/or C” or “A, B, C, or any combination thereof” can mean “A individually; B individually; C individually; A and B; B and C; A and C; and A, B, and C.” The term “or” can be used conjunctively or disjunctively, unless the context specifically refers to a disjunctive use.

The term “about” or “approximately” can mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, within 5-fold, and more preferably within 2-fold, of a value. Where particular values are described in the application and claims, unless otherwise stated the term “about” meaning within an acceptable error range for the particular value should be assumed.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures. To facilitate an understanding of the present disclosure, a number of terms and phrases are defined below.

Major Histocompatibility Complex or “MHC” is a cluster of genes that plays a role in control of the cellular interactions responsible for physiologic immune responses. In humans, the MHC complex is also known as the human leukocyte antigen (HLA) complex. For a detailed description of the MHC and HLA complexes, see, Paul, Fundamental Immunology, 3^{rd} Ed., Raven Press, New York (1993). “Proteins or molecules of the major histocompatibility complex (MHC)”, “MHC molecules”, “MHC proteins” or “HLA proteins” are to be understood as meaning proteins capable of binding peptides resulting from the proteolytic cleavage of protein antigens and representing potential lymphocyte epitopes, (e.g., T cell epitope and B cell epitope) transporting them to the cell surface and presenting them there to specific cells, in particular cytotoxic T-lymphocytes, T-helper cells, or B cells. The major histocompatibility complex in the genome comprises the genetic region whose gene products expressed on the cell surface are important for binding and presenting endogenous and/or foreign antigens and thus for regulating immunological processes. The major histocompatibility complex is classified into two gene groups coding for different proteins, namely molecules of MHC class I and molecules of MHC class II. The cellular biology and the expression patterns of the two MHC classes are adapted to these different roles.

Human Leukocyte Antigen or “HLA” is a human class I or class II Major Histocompatibility Complex (MHC) protein (see, e.g., Stites, et al., Immunology, 8th Ed., Lange Publishing, Los Altos, Calif. (1994).

“Polypeptide”, “peptide” and their grammatical equivalents as used herein refer to a polymer of amino acid residues. The polymer of amino acid residues is a series of amino acid residues connected one to the other, typically by peptide bonds between the α-amino and carboxyl groups of adjacent amino acid residues. Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The present disclosure further contemplates that expression of polypeptides described herein in an engineered cell can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitination, addition of pyrrolidone carboxylic acid, formation of disulfide bridges, sulfation, myristoylation, palmitoylation, isoprenylation, farnesylation, geranylation, glypiation, lipoylation and iodination.

An immunogenic peptide or an immunogenic epitope or peptide epitope is a peptide that comprises an allele-specific motif such that the peptide will bind an HLA molecule and induce a cell-mediated or humoral response, for example, cytotoxic T lymphocyte (CTL (e.g., CD8⁺)), helper T lymphocyte (Th (e.g., CD4⁺)) and/or B lymphocyte response. Thus, immunogenic peptides described herein are capable of binding to an appropriate HLA molecule and thereafter inducing a CTL (cytotoxic) response, or a HTL (and humoral) response, to the peptide.

Neoantigens are a class of tumor antigens which arise from tumor-specific changes in proteins. Neoantigens encompass, but are not limited to, tumor antigens which arise from, for example, a cancer-specific mutation in a subject, which includes but is not limited to a substitution mutation, a frame shift mutation, a gene fusion, an in-frame deletion or insertion. Neoantigens can also include endogenous retroviral polypeptides and polypeptides containing tumor-specific mutations that are overexpressed.

The term “peptide” and “polypeptide” are used interchangeably, and refer to a series of residues, typically L-amino acids, connected one to the other, typically by peptide bonds between the α-amino and carboxyl groups of adjacent amino acids. In some embodiments, a polypeptide comprises a single translation product comprising multiple discrete epitopes. In some embodiments, a polypeptide comprises a single translation product comprising a single discrete peptide such as an epitope or neo-epitope; and one or more additional non-discrete elements such as one or more additional scaffold proteins. In some embodiments, a polypeptide comprises multiple epitopes. In some embodiments a polypeptide can be a branched multi-peptide structure. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described. In some embodiments, a peptide or polypeptide comprises at least one flanking sequence. The term “flanking sequence” as used herein refers to a fragment or region of the neoantigen peptide that is not a part of the neoepitope. The terms “mutant polypeptide”, “neoantigen polypeptide”, “neoantigenic polypeptide”, “mutant peptide”, “neoantigen peptide” and “neoantigenic peptide”, are used interchangeably and refer to a peptide or polypeptide containing a mutation.

The term “residue” refers to an amino acid residue or amino acid mimetic residue incorporated into a peptide or protein by an amide bond or amide bond mimetic, or nucleic acid (DNA or RNA) that encodes the amino acid or amino acid mimetic.

A “neoepitope”, “tumor specific neoepitope” or “tumor antigen” refers to an epitope or antigenic determinant region that is not present in a reference, such as a non-diseased cell, e.g., a non-cancerous cell or a germline cell, but is found in a diseased cell, e.g., a cancer cell. The term “neoepitope” as used herein refers to an antigenic determinant region within the peptide or neoantigenic peptide. A neoepitope may comprise at least one “anchor residue” and at least one “anchor residue flanking region.” A neoepitope may further comprise a “separation region.” The term “anchor residue” refers to an amino acid residue that binds to specific pockets on HLAs, resulting in specificity of interactions with HLAs. In some cases, an anchor residue may be at a canonical anchor position. In other cases, an anchor residue may be at a non-canonical anchor position. Neoepitopes may bind to HLA molecules through primary and secondary anchor residues protruding into the pockets in the peptide-binding grooves. In the peptide-binding grooves, specific amino acids compose pockets that accommodate the corresponding side chains of the anchor residues of the presented neoepitopes. Peptide-binding preferences exist among different alleles of both of HLA I and HLA II molecules. HLA class I molecules bind short neoepitopes, whose N- and C-terminal ends are anchored into the pockets located at the ends of the neoepitope binding groove. While the majority of the HLA class I binding neoepitopes are of about 9 amino acids, longer neoepitopes can be accommodated by the bulging of their central portion, resulting in binding neoepitopes of about 8 to 12 amino acids. Neoepitopes binding to HLA class II proteins are not constrained in size and can vary from about 16 to 25 amino acids. The neoepitope binding groove in the HLA class II molecules is open at both ends, which enables binding of peptides with relatively longer length. Though the core segment of about 9 amino acid residues contributes the most to the recognition of the neoepitope, the anchor residues flanking this regions are also important for the specificity of the peptide to the HLA class II allele. In some cases, the anchor residue flanking the core region that contributes to the specificity of the peptide is present at the N-terminus of the core region residues. In another case, the anchor residue flanking the core region is present at the C-terminus of the core region residues. In yet another case, the anchor residues flanking the core region that contribute to the specificity of the peptide to HLA is both at the N-terminus and at the C-terminus of the core region residues

A “reference” can be used to correlate and compare the results obtained in the methods of the present disclosure from a tumor specimen. Typically, the “reference” may be obtained on the basis of one or more normal specimens, in particular specimens which are not affected by a cancer disease, either obtained from a patient or one or more different individuals, for example, healthy individuals, in particular individuals of the same species. A “reference” can be determined empirically by testing a sufficiently large number of normal specimens.

An “epitope” is the collective features of a molecule, such as primary, secondary and tertiary peptide structure, and charge, that together form a site recognized by, for example, an immunoglobulin, T cell receptor, HLA molecule, or chimeric antigen receptor. Alternatively, an epitope can be defined as a set of amino acid residues which is involved in recognition by a particular immunoglobulin, or in the context of T cells, those residues necessary for recognition by T cell receptor proteins, chimeric antigen receptors, and/or Major Histocompatibility Complex (MHC) receptors. A “T cell epitope” is to be understood as meaning a peptide sequence which can be bound by the MHC molecules of class I or II in the form of a peptide-presenting MHC molecule or MHC complex and then, in this form, be recognized and bound by T cells, such as T-lymphocytes or T-helper cells. Epitopes can be prepared by isolation from a natural source, or they can be synthesized according to standard protocols in the art. Synthetic epitopes can comprise artificial amino acid residues, “amino acid mimetics,” such as D isomers of naturally-occurring L amino acid residues or non-naturally-occurring amino acid residues such as cyclohexylalanine. Throughout this disclosure, epitopes may be referred to in some cases as peptides or peptide epitopes. It is to be appreciated that proteins or peptides that comprise an epitope or an analog described herein as well as additional amino acid(s) are still within the bounds of the present disclosure. In certain embodiments, the peptide comprises a fragment of an antigen. In certain embodiments, there is a limitation on the length of a peptide of the present disclosure. The embodiment that is length-limited occurs when the protein or peptide comprising an epitope described herein comprises a region (i.e., a contiguous series of amino acid residues) having 100% identity with a native sequence.

The nomenclature used to describe peptides or proteins follows the conventional practice wherein the amino group is presented to the left (the amino- or N-terminus) and the carboxyl group to the right (the carboxy- or C-terminus) of each amino acid residue. When amino acid residue positions are referred to in a peptide epitope they are numbered in an amino to carboxyl direction with position one being the residue located at the amino terminal end of the epitope, or the peptide or protein of which it can be a part. In the formula representing selected specific embodiments of the present disclosure, the amino- and carboxyl-terminal groups, although not specifically shown, are in the form they would assume at physiologic pH values, unless otherwise specified. In the amino acid structure formula, each residue is generally represented by standard three letter or single letter designations However, when three letter symbols or full names are used without capitals, they can refer to L amino acid residues. Glycine has no asymmetric carbon atom and is simply referred to as “Gly” or “G”. The amino acid sequences of peptides set forth herein are generally designated using the standard single letter symbol. (A, Alanine; C, Cysteine; D, Aspartic Acid; E, Glutamic Acid; F, Phenylalanine; G, Glycine; H, Histidine; I, Isoleucine; K, Lysine; L, Leucine; M, Methionine; N, Asparagine; P, Proline; Q, Glutamine; R, Arginine; S, Serine; T, Threonine; V, Valine; W, Tryptophan; and Y, Tyrosine.)

The term mutation refers to a change in the nucleic acid sequence (nucleotide substitution, addition or deletion) compared to a reference. A somatic mutation is genetic alteration acquired by a cell that can be passed to the progeny of the mutated cell in the course of cell division. Somatic mutations differ from germ line mutations, which are inherited genetic alterations that occur in the germ cells (i.e., sperm and eggs). In some embodiments, a mutation is a non-synonymous mutation. The term non-synonymous mutation refers to a mutation, for example, a nucleotide substitution, which does result in an amino acid change such as an amino acid substitution in the translation product. A frameshift occurs when a mutation disrupts the normal phase of a gene's codon periodicity (also known as “reading frame”), resulting in the translation of a non-native protein sequence. It is possible for different mutations in a gene to achieve the same altered reading frame.

A conservative amino acid substitution is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Methods of identifying nucleotide and amino acid conservative substitutions which do not eliminate peptide function are well-known in the art.

As used herein, the term affinity refers to a measure of the strength of binding between two members of a binding pair, for example, an HLA-binding peptide and a class I or II HLA. K_{D} is the dissociation constant and has units of molarity. The affinity constant is the inverse of the dissociation constant. An affinity constant is sometimes used as a generic term to describe this chemical entity. It is a direct measure of the energy of binding. Affinity may be determined experimentally, for example by surface plasmon resonance (SPR) using commercially available Biacore SPR units. Affinity may also be expressed as the inhibitory concentration 50 (IC₅₀), that concentration at which 50% of the peptide is displaced. Likewise, ln(IC₅₀) refers to the natural log of the IC₅₀. K_{off} refers to the off-rate constant, for example, for dissociation of an HLA-binding peptide and a class I or II HLA. Throughout this disclosure, “binding data” results can be expressed in terms of “IC₅₀.” IC₅₀ is the concentration of the tested peptide in a binding assay at which 50% inhibition of binding of a labeled reference peptide is observed. Given the conditions in which the assays are run (i.e., limiting HLA protein and labeled reference peptide concentrations), these values approximate K_{D} values. Assays for determining binding are well known in the art and are described in detail, for example, in PCT publications WO 94/20127 and WO 94/03205, and other publications such Sidney et al., Current Protocols in Immunology 18.3.1 (1998); Sidney, et al., J. Immunol. 154:247 (1995); and Sette, et al., Mol. Immunol. 31:813 (1994). Alternatively, binding can be expressed relative to binding by a reference standard peptide. For example, can be based on its IC₅₀, relative to the IC₅₀ of a reference standard peptide. Binding can also be determined using other assay systems including those using: live cells (e.g., Ceppellini et al., Nature 339:392 (1989); Christnick et al., Nature 352:67 (1991); Busch et al., Int. Immunol. 2:443 (1990); Hill et al., J. Immunol. 147:189 (1991); del Guercio et al., J. Immunol. 154:685 (1995)), cell free systems using detergent lysates (e.g., Cerundolo et al., J. Immunol. 21:2069 (1991)), immobilized purified MHC (e.g., Hill et al., J. Immunol. 152, 2890 (1994); Marshall et al., J. Immunol. 152:4946 (1994)), ELISA systems (e.g., Reay et al., EMBO J. 11:2829 (1992)), surface plasmon resonance (e.g., Khilko et al., J. Biol. Chem. 268:15425 (1993)); high flux soluble phase assays (Hammer et al., J. Exp. Med. 180:2353 (1994)), and measurement of class I MHC stabilization or assembly (e.g., Ljunggren et al., Nature 346:476 (1990); Schumacher et al., Cell 62:563 (1990); Townsend et al., Cell 62:285 (1990); Parker et al., J. Immunol. 149:1896 (1992)). “Cross-reactive binding” is evident in some peptides, that is, the peptides exhibit promiscuous binding to more than one HLA molecule.

The term “derived” and its grammatical equivalents when used to discuss an epitope is a synonym for “prepared” and its grammatical equivalents. A derived epitope can be isolated from a natural source, or it can be synthesized according to standard protocols in the art. Synthetic epitopes can comprise artificial amino acid residues “amino acid mimetics,” such as D isomers of natural occurring L amino acid residues or non-natural amino acid residues such as cyclohexylalanine. A derived or prepared epitope can be an analog of a native epitope.

A “diluent” includes sterile liquids, such as water and oils, including those of petroleum, animal, vegetable or synthetic origin, such as peanut oil, soybean oil, mineral oil, sesame oil and the like. Water is also a diluent for pharmaceutical compositions. Saline solutions and aqueous dextrose and glycerol solutions can also be employed as diluents, for example, in injectable solutions.

A “native” or a “wild type” sequence refers to a sequence found in nature. Such a sequence can comprise a longer sequence in nature.

A “receptor” is to be understood as meaning a biological molecule or a molecule grouping capable of binding a ligand. A receptor may serve, to transmit information in a cell, a cell formation or an organism. The receptor comprises at least one receptor unit, for example, where each receptor unit may consist of a protein molecule. The receptor has a structure which complements that of a ligand and may complex the ligand as a binding partner. The information is transmitted in particular by conformational changes of the receptor following complexation of the ligand on the surface of a cell. In some embodiments, a receptor is to be understood as meaning in particular proteins of MHC classes I and II capable of forming a receptor/ligand complex with a ligand, in particular a peptide or peptide fragment of suitable length.

A “ligand” is to be understood as meaning a molecule which has a structure complementary to that of a receptor and is capable of forming a complex with this receptor. In some embodiments, a ligand is to be understood as meaning a peptide or peptide fragment which has a suitable length and suitable binding motifs in its amino acid sequence, so that the peptide or peptide fragment is capable of forming a complex with proteins of MHC class I or MHC class II.

In some embodiments, a “receptor/ligand complex” is also to be understood as meaning a “receptor/peptide complex” or “receptor/peptide fragment complex”, including a peptide- or peptide fragment-presenting MHC molecule of class I or of class II.

“Synthetic peptide” refers to a peptide that is obtained from a non-natural source, e.g., is man-made. Such peptides can be produced using such methods as chemical synthesis or recombinant DNA technology. “Synthetic peptides” include “fusion proteins.”

The term “motif” refers to a pattern of residues in an amino acid sequence of defined length, for example, a peptide of less than about 15 amino acid residues in length, or less than about 13 amino acid residues in length, for example, from about 8 to about 13 amino acid residues (e.g., 8, 9, 10, 11, 12, or 13) for a class I HLA motif and from about 6 to about 25 amino acid residues (e.g., 6, 7, 8, 9, 10, 11, 12, 13, 14, 15, 16, 17, 18, 19, 20, 21, 22, 23, 24, or 25) for a class II HLA motif, which is recognized by a particular HLA molecule. Motifs are typically different for each HLA protein encoded by a given human HLA allele. These motifs differ in their pattern of the primary and secondary anchor residues. In some embodiments, an MHC class I motif identifies a peptide of 9, 10, or 11 amino acid residues in length.

The term “naturally occurring” and its grammatical equivalents as used herein refer to the fact that an object can be found in nature. For example, a peptide or nucleic acid that is present in an organism (including viruses) and can be isolated from a source in nature and which has not been intentionally modified by man in the laboratory is naturally occurring.

According to the present disclosure, the term “vaccine” relates to a pharmaceutical preparation (pharmaceutical composition) or product that upon administration induces an immune response, for example, a cellular or humoral immune response, which recognizes and attacks a pathogen or a diseased cell such as a cancer cell. A vaccine may be used for the prevention or treatment of a disease. The term “individualized cancer vaccine” or “personalized cancer vaccine” concerns a particular cancer patient and means that a cancer vaccine is adapted to the needs or special circumstances of an individual cancer patient.

A “protective immune response” or “therapeutic immune response” refers to a CTL and/or an HTL response to an antigen derived from a pathogenic antigen (e.g., a tumor antigen), which in some way prevents or at least partially arrests disease symptoms, side effects or progression. The immune response can also include an antibody response which has been facilitated by the stimulation of helper T cells.

“Antigen processing” or “processing” and its grammatical equivalents refers to the degradation of a polypeptide or antigen into procession products, which are fragments of said polypeptide or antigen (e.g., the degradation of a polypeptide into peptides) and the association of one or more of these fragments (e.g., via binding) with MHC molecules for presentation by cells, for example, antigen presenting cells, to specific T cells.

“Antigen presenting cells” (APC) are cells which present peptide fragments of protein antigens in association with MHC molecules on their cell surface. Some APCs may activate antigen specific T cells. Professional antigen-presenting cells are very efficient at internalizing antigen, either by phagocytosis or by receptor-mediated endocytosis, and then displaying a fragment of the antigen, bound to a class II MHC molecule, on their membrane. The T cell recognizes and interacts with the antigen-class II MHC molecule complex on the membrane of the antigen presenting cell. An additional co-stimulatory signal is then produced by the antigen presenting cell, leading to activation of the T cell. The expression of co-stimulatory molecules is a defining feature of professional antigen-presenting cells. The main types of professional antigen-presenting cells are dendritic cells, which have the broadest range of antigen presentation, and are probably the most important antigen presenting cells, macrophages, B-cells, and certain activated epithelial cells. Dendritic cells (DCs) are leukocyte populations that present antigens captured in peripheral tissues to T cells via both MHC class II and I antigen presentation pathways. It is well known that dendritic cells are potent inducers of immune responses and the activation of these cells is a critical step for the induction of antitumoral immunity. Dendritic cells are conveniently categorized as “immature” and “mature” cells, which can be used as a simple way to discriminate between two well characterized phenotypes. However, this nomenclature should not be construed to exclude all possible intermediate stages of differentiation. Immature dendritic cells are characterized as antigen presenting cells with a high capacity for antigen uptake and processing, which correlates with the high expression of Fc receptor (FcR) and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1 BB).

The terms “identical” and its grammatical equivalents as used herein or “sequence identity” in the context of two nucleic acid sequences or amino acid sequences of polypeptides refers to the residues in the two sequences which are the same when aligned for maximum correspondence over a specified comparison window. An exemplary “comparison window”, as used herein, refers to a segment of at least about 20 contiguous positions, usually about 50 to about 200, more usually about 100 to about 150 in which a sequence may be compared to a reference sequence of the same number of contiguous positions after the two sequences are aligned optimally. Methods of alignment of sequences for comparison are well-known in the art. Optimal alignment of sequences for comparison may be conducted by the local homology algorithm of Smith and Waterman, Adv. Appl. Math., 2:482 (1981); by the alignment algorithm of Needleman and Wunsch, J. Mol. Biol., 48:443 (1970); by the search for similarity method of Pearson and Lipman, Proc. Nat. Acad. Sci. U.S.A., 85:2444 (1988); by computerized implementations of these algorithms (including, but not limited to CLUSTAL in the PC/Gene program by Intelligentics, Mountain View Calif., GAP, BESTFIT, BLAST, FASTA, and TFASTA in the Wisconsin Genetics Software Package, Genetics Computer Group (GCG), 575 Science Dr., Madison, Wis., U.S.A.); the CLUSTAL program is well described by Higgins and Sharp, Gene, 73:237-244 (1988) and Higgins and Sharp, CABIOS, 5:151-153 (1989); Corpet et al., Nucleic Acids Res., 16:10881-10890 (1988); Huang et al., Computer Applications in the Biosciences, 8:155-165 (1992); and Pearson et al., Methods in Molecular Biology, 24:307-331 (1994). Alignment is also often performed by inspection and manual alignment. In one class of embodiments, the polypeptides herein have at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a reference polypeptide, or a fragment thereof, e.g., as measured by BLASTP (or CLUSTAL, or any other available alignment software) using default parameters. Similarly, nucleic acids can also be described with reference to a starting nucleic acid, e.g., they can have 50%, 60%, 70%, 75%, 80%, 85%, 90%, 98%, 99% or 100% sequence identity to a reference nucleic acid or a fragment thereof, e.g., as measured by BLASTN (or CLUSTAL, or any other available alignment software) using default parameters. When one molecule is said to have certain percentage of sequence identity with a larger molecule, it means that when the two molecules are optimally aligned, said percentage of residues in the smaller molecule finds a match residue in the larger molecule in accordance with the order by which the two molecules are optimally aligned.

“Substantially identical” and its grammatical equivalents as applied to nucleic acid or amino acid sequences mean that a nucleic acid or amino acid sequence comprises a sequence that has at least 90% sequence identity or more, at least 95%, at least 98% and at least 99%, compared to a reference sequence using the programs described above, e.g., BLAST, using standard parameters. For example, the BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1992)). Percentage of sequence identity is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. In embodiments, the substantial identity exists over a region of the sequences that is at least about 50 residues in length, over a region of at least about 100 residues, and in embodiments, the sequences are substantially identical over at least about 150 residues. In embodiments, the sequences are substantially identical over the entire length of the coding regions.

Vector generally means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure. In some embodiments, an “isolated polynucleotide” encompasses a PCR or quantitative PCR reaction comprising the polynucleotide amplified in the PCR or quantitative PCR reaction.

The term “isolated”, “biologically pure” or their grammatical equivalents refers to material which is substantially or essentially free from components which normally accompany the material as it is found in its native state. Thus, isolated peptides described herein do not contain some or all of the materials normally associated with the peptides in their in situ environment. An “isolated” epitope refers to an epitope that does not include the whole sequence of the antigen from which the epitope was derived. Typically the “isolated” epitope does not have attached thereto additional amino acid residues that result in a sequence that has 100% identity over the entire length of a native sequence. The native sequence can be a sequence such as a tumor-associated antigen from which the epitope is derived. Thus, the term “isolated” means that the material is removed from its original environment (e.g., the natural environment if it is naturally occurring). An “isolated” nucleic acid is a nucleic acid removed from its natural environment. For example, a naturally-occurring polynucleotide or peptide present in a living animal is not isolated, but the same polynucleotide or peptide, separated from some or all of the coexisting materials in the natural system, is isolated. Such a polynucleotide could be part of a vector, and/or such a polynucleotide or peptide could be part of a composition, and still be “isolated” in that such vector or composition is not part of its natural environment. Isolated RNA molecules include in vivo or in vitro RNA transcripts of the DNA molecules described herein, and further include such molecules produced synthetically.

The term “substantially purified” and its grammatical equivalents as used herein refer to a nucleic acid sequence, polypeptide, protein or other compound which is essentially free, i.e., is more than about 50% free of, more than about 70% free of, more than about 90% free of, the polynucleotides, proteins, polypeptides and other molecules that the nucleic acid, polypeptide, protein or other compound is naturally associated with.

The term “substantially pure” as used herein refers to material which is at least 50% pure (i.e., free from contaminants), at least 90% pure, at least 95% pure, at least 98% pure, or at least 99% pure.

The terms “polynucleotide”, “nucleotide”, “nucleic acid”, “polynucleic acid” or “oligonucleotide” and their grammatical equivalents are used interchangeably herein and refer to polymers of nucleotides of any length, and include DNA and RNA, for example, mRNA. Thus, these terms include double and single stranded DNA, triplex DNA, as well as double and single stranded RNA. It also includes modified, for example, by methylation and/or by capping, and unmodified forms of the polynucleotide. The term is also meant to include molecules that include non-naturally occurring or synthetic nucleotides as well as nucleotide analogs. The nucleic acid sequences and vectors disclosed or contemplated herein may be introduced into a cell by, for example, transfection, transformation, or transduction. The nucleotides can be deoxyribonucleotides, ribonucleotides, modified nucleotides or bases, and/or their analogs, or any substrate that can be incorporated into a polymer by DNA or RNA polymerase. In some embodiments, the polynucleotide and nucleic acid can be in vitro transcribed mRNA. In some embodiments, the polynucleotide that is administered using the methods of the present disclosure is mRNA.

Transfection, transformation, or transduction as generally refer to the introduction of one or more exogenous polynucleotides into a host cell by using physical or chemical methods. Many transfection techniques are known in the art and include, for example, calcium phosphate DNA co-precipitation (see, e.g., Murray E. J. (ed.), Methods in Molecular Biology, Vol. 7, Gene Transfer and Expression Protocols, Humana Press (1991)); DEAE-dextran; electroporation; cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)). Phage or viral vectors can be introduced into host cells, after growth of infectious particles in suitable packaging cells, many of which are commercially available.

Nucleic acids and/or nucleic acid sequences are “homologous” when they are derived, naturally or artificially, from a common ancestral nucleic acid or nucleic acid sequence. Proteins and/or protein sequences are “homologous” when their encoding DNAs are derived, naturally or artificially, from a common ancestral nucleic acid or nucleic acid sequence. The homologous molecules can be termed homologs. For example, any naturally occurring proteins, as described herein, can be modified by any available mutagenesis method. When expressed, this mutagenized nucleic acid encodes a polypeptide that is homologous to the protein encoded by the original nucleic acid. Homology is generally inferred from sequence identity between two or more nucleic acids or proteins (or sequences thereof). The precise percentage of identity between sequences that is useful in establishing homology varies with the nucleic acid and protein at issue, but as little as 25% sequence identity is routinely used to establish homology. Higher levels of sequence identity, e.g., 30%, 40%, 50%, 60%, 70%, 80%, 90%, 95% or 99% or more can also be used to establish homology. Methods for determining sequence identity percentages (e.g., BLASTP and BLASTN using default parameters) are described herein and are generally available.

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The terms “effective amount” or “therapeutically effective amount” or “therapeutic effect” refer to an amount of a therapeutic effective to “treat” a disease or disorder in a subject or mammal. The therapeutically effective amount of a drug has a therapeutic effect and as such can prevent the development of a disease or disorder; slow down the development of a disease or disorder; slow down the progression of a disease or disorder; relieve to some extent one or more of the symptoms associated with a disease or disorder; reduce morbidity and mortality; improve quality of life; or a combination of such effects.

The terms “treating” or “treatment” or “to treat” or “alleviating” or “to alleviate” refer to both (1) therapeutic measures that cure, slow down, lessen symptoms of, and/or halt progression of a diagnosed pathologic condition or disorder; and (2) prophylactic or preventative measures that prevent or slow the development of a targeted pathologic condition or disorder. Thus, those in need of treatment include those already with the disorder; those prone to have the disorder; and those in whom the disorder is to be prevented.

“Pharmaceutically acceptable” refers to a generally non-toxic, inert, and/or physiologically compatible composition or component of a composition.

A “pharmaceutical excipient” or “excipient” comprises a material such as an adjuvant, a carrier, pH-adjusting and buffering agents, tonicity adjusting agents, wetting agents, preservatives, and the like. A “pharmaceutical excipient” is an excipient which is pharmaceutically acceptable.

As used herein, the term “scaffold domain,” “scaffolding domain,” and “scaffold polypeptide” are used interchangeably.

In one aspect, described herein are fusion polypeptides comprising multiple epitopes or antigens fused to scaffold proteins. Epitopes are short peptides that can be approximately 5-50 amino acids in length. The fusion polypeptides, or fusion polyproteins or “polybodies” can be generated by combining multiple epitopes into one molecule. Polybodies are useful for generating a therapeutic comprising the epitopes, where the epitopes are the active therapeutic ingredients, e.g., for immunotherapy. Whereas an epitope alone can be rapidly degraded inside the living system without achieving its expected result, the polybody form can be designed to provide advantages over the single epitope form. In polybodies, the scaffold protein stabilizes neoantigens; enhance solubility of epitopes; increase the molecular weight, e.g., greater than 50 kDa to enable lymph node targeting and retention and can be designed to perform specific tissue or cell targeting, or introducing tags for protein purification or identification.

From a manufacturing standpoint, polybody reduces batch manufacturing of single peptides. Absence of post-translational modification allows less expensive rapid recombinant production of scaffolds in E. coli. Scaffolds are relatively small in size with no known toxicities. In some embodiments, scaffold proteins are human proteins, thereby ensuring lack of antigenicity. In some embodiments, functional scaffolds can be incorporated, such as for dendritic cell targeting or activation. Ubiquitin scaffold may potentiate proteasome degradation and processing of antigens. In some embodiments, a scaffold protein may be further modified, for example to include a cell targeting moiety.

### Fusion Polypeptides

In one aspect, disclosed herein are fusion polypeptides comprising one or more antigen polypeptides and one or more scaffold polypeptides. Also disclosed herein are nucleic acid molecules encoding the fusion polypeptides and compositions comprising the fusion polypeptides or the nucleic acid molecules. In some embodiments, the fusion polypeptide comprises a polypeptide sequence, said sequence comprising one or more antigen polypeptides sequences and one or more scaffold polypeptides sequences. In some embodiments, the fusion polypeptide comprises two or more scaffold polypeptides sequences. In some embodiments, each of the scaffold polypeptides sequences comprises a human polypeptide sequence, a fragment thereof or a variant thereof. In some embodiments, a molecular mass of the two or more scaffold polypeptides sequences is more than 11 kDa. In some embodiments, each of the two or more scaffold polypeptides sequences comprises at least 21 amino acid residues. In some embodiments, the scaffold polypeptides are selected from Stefin A, Titin-127, a fragment thereof, and a variant thereof. In some embodiments, at least one of the scaffold polypeptides sequences is connected to at least one of the one or more antigen polypeptides sequences through one or more linker sequences. In some embodiments, the fusion polypeptide is configured to facilitate a cleavage of the linker, a cleavage of at least one of the one or more antigen polypeptides, or presentation of at least one of the one or more antigen polypeptides. In some embodiments, the antigen polypeptides are cancer antigens.

In some embodiments, a molecular mass of the fusion polypeptide is at least 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, or 200 kDa. In some embodiments, a molecular mass of the fusion polypeptide is at least 75 kDa. In some embodiments, a molecular mass of the fusion polypeptide is at most 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, 200 kDa, or 500 kDa. In some embodiments, a molecular mass of the fusion polypeptide is from about 50 to 80 kDa. In some embodiments, a molecular mass of the fusion polypeptide is from about 70 to 80 kDa. The fusion polypeptide can be configured to take a linear or branched format. In some embodiments, the fusion polypeptide is in a linear format. In some embodiments, the fusion polypeptide has multiple branches.

The one or more scaffold polypeptides can be linked to the remaining sequences of the fusion polypeptide or among themselves in various ways. For example, the scaffold polypeptides can be linked via the N- or C-terminus or via an insertion between the N- and C-terminus. In some embodiments, at least one of the scaffold polypeptides sequences is linked, via its N-terminus, to the remaining sequences of the fusion polypeptide. In some embodiments, at least one of the scaffold polypeptides sequences is linked, via its C-terminus, to the remaining sequences of the fusion polypeptide. In some embodiments, each of the one or more scaffold polypeptides is linked to the remaining sequence of the fusion polypeptide via its N-terminus, C-terminus, or both terminuses. In some embodiments, at least two of the scaffold polypeptides sequences are uninterrupted by the antigen sequences or linker sequences. In some embodiments, the scaffold polypeptides are not linked by an insertion between their respective N- and C-terminus.

In some embodiments, the fusion polypeptide comprises, in the N-terminus to C-terminus direction, a polypeptide sequence having a structure of Formula (Ia),

Aₙ-Sₘ, or Sₘ-Aₙ Formula (Ia),

wherein each S independently represents a scaffold polypeptide sequence and each A independently represents an antigen polypeptide sequence, and wherein m is an integer equal or greater than 1, and n is an integer equal or greater than 1. In some embodiments, each of m and n is an integer independently selected from 1 to 10. In some embodiments, each of m and n is an integer independently selected from 1 to 5. In some embodiments, each of m and n is an integer independently selected from 1 to 3. In some embodiments, each of m and n is independently 1, 2, 3, 4, 5, 6, 7, 8, 9, or 10. In some embodiments, m is 1, and n is 2. In some embodiments, n is 1, and m is 2. In some embodiments, the fusion polypeptide comprises a polypeptide sequence, in the N-terminus to C-terminus direction, having a structure of A-A-S, A-A-A-S, A-A-A-A-S, A-A-A-A-A-S, A-A-A-A-A-A-S, or A-A-A-A-A-A-A-S. In some embodiments, the fusion polypeptide comprises a polypeptide sequence, in the N-terminus to C-terminus direction, having a structure of S-A-A, S-A-A-A, S-A-A-A-A, S-A-A-A-A-A, S-A-A-A-A-A-A, or S-A-A-A-A-A-A-A. In some embodiments, the fusion polypeptide comprises a polypeptide sequence, in the N-terminus to C-terminus direction, having a structure of A-S, A-S-S, A-S-S-S, A-S-S-S-S, A-S-S-S-S-S, or A-S-S-S-S-S-S. In some embodiments, the fusion polypeptide comprises a polypeptide sequence, in the N-terminus to C-terminus direction, having a structure of S-A, S-S-A, S-S-S-A, S-S-S-S-A, S-S-S-S-S-A, or S-S-S-S-S-S-A.

In some embodiments, the fusion polypeptide comprises, in the N-terminus to C-terminus direction, a polypeptide sequence having a structure of Formula (Ib),

Sₒ-Aₙ-Sₘ Formula (Ib),

wherein each S independently represents a scaffold polypeptide sequence and each A independently represents an antigen polypeptide sequence, and wherein each of o, n, and m is independently an integer equal or greater than 1. In some embodiments, each of o, n, and m is independently 1, 2, 3, 4, 5, 6, 7, 8, 9, or 10. In some embodiments, each of o, m, and n is an integer independently selected from 1 to 5. In some embodiments, o is 1 or 2, m is 1 or 2, and n is an integer selected from 1 to 5. In some embodiments, o and m equal to 1, and n is an integer from 1 to 5. In some embodiments, the fusion polypeptide comprises a polypeptide sequence, in the N-terminus to C-terminus direction, having a structure of S-A-S, A-S-A-S, S-A-S-A, S-A-S-A-S, S-A-S-A-S-A-S, S-A-S-A-S-A-S-A-S, or S-A-S-A-S-A-S-A-S-A-S. In some embodiments, the fusion polypeptide comprises a polypeptide sequence, in the N-terminus to C-terminus direction, having a structure of S-A-A-S, S-A-A-A-S, S-A-A-A-A-S, S-A-A-A-A-A-S, S-A-A-A-A-A-A-S, or S-A-A-A-A-A-A-A-S.

In some embodiments, the fusion polypeptide comprises, in the N-terminus to C-terminus direction, a polypeptide sequence having a structure of Formula (IIa),

(L-A-L)ₙ-Sₘ, or Sₘ-(L-A-L)ₙ Formula (IIa),

wherein each S independently represents a scaffold polypeptide sequence, each A independently represents an antigen polypeptide sequence, and each L independently represents a linker sequence or is absent, and wherein m is an integer equal or greater than 1, and n is an integer equal or greater than 1. In some embodiments of Formula (IIa), m is an integer equal or greater than 2. In some embodiments of Formula (IIa), n is an integer selected from 1 to 5, and m is 2. In some embodiments, the fusion polypeptide comprises a polypeptide sequence, in the N-terminus to C-terminus direction, having a structure of A-L-S, A-L-A-L-S, A-L-A-L-A-L-S, S-L-A, S-L-A-L-A, or S-L-A-L-A-L-A.

In some embodiments, the fusion polypeptide comprises, in the N-terminus to C-terminus direction, a polypeptide sequence having a structure of Formula (IIb),

Sₒ-(L-A-L)ₙ-Sₘ Formula (IIb),

wherein each S independently represents a scaffold polypeptide sequence, each A independently represents an antigen polypeptide sequence, and each L independently represents a linker sequence or is absent, and wherein each of o, n, and m is independently an integer equal or greater than 1. In some embodiments, o is 1 or 2, m is 1 or 2, and n is an integer selected from 1 to 5. In some embodiments, the fusion polypeptide comprises a polypeptide sequence, in the N-terminus to C-terminus direction, having a structure of S-L-A-L-S, S-A-L-S-L-A-L-S, S-L-A-L-S-L-A, S-L-A-L-S-L-A-L-S, S-L-A-L-S-L-A-L-S-L-A-L-S, S-L-A-L-S-L-A-L-S-L-A-L-S-L-A-S, or S-L-A-L-S-L-A-L-S-L-A-L-S-L-A-S-L-A-L-S.

In some embodiments, the scaffold polypeptides and the antigen polypeptides are situated in the fusion polypeptide in an alternating fashion, with or without a linker sequence in between. For example, in some embodiments, the fusion polypeptide comprises a polypeptide sequence, in the N-terminus to C-terminus direction, having a structure of S-A-L-S-L-A-L-S, A-L-S-L-A-L-S-L-A, A-S-L-A-L-S-L-A-L-S, S-L-A-L-S-L-A-L-S-L-A-L-S-A, A-S-A-S-L-A-L-S, or S-L-A-L-S-L-A-L-S-A-S-A. In some embodiments, each of the scaffold polypeptides sequences is connected to one or two of the antigen polypeptides sequences through at least one of the linker sequences.

The described fusion polypeptide can lead to an improved lymph node accumulation or retention compared to the corresponding polypeptide lacking the scaffold domains. In some embodiments, an accumulation of the fusion polypeptide in lymph node following administration to a subject is at least 1.1, 1.2, 1.3, 1.4, 1.5, 1.6, 1.7, 1.8, 1.9, 2, 3, 4, 5, 6, 7, 8, 9, or 10 fold higher than an accumulation of its corresponding polypeptide lacking the scaffold polypeptides, as measured by an average radiant efficiency. In some embodiments, an accumulation of the fusion polypeptide in lymph node following administration to a subject is at least 1.1, 2, 3, 4, 5, 6, 7, 8, 9, or 10 fold higher than an accumulation of its corresponding polypeptide lacking the scaffold polypeptides, as measured by an average radiant efficiency. For example, the lymph node accumulation can be measured according to the method described in Example 3.1.

The described fusion polypeptide can have an improved solubility in an aqueous solvent, compared to the corresponding polypeptide lacking the scaffold domains. In some embodiments, the solubility of the fusion polypeptide in an aqueous solvent is at least 1.1, 2, 3, 4, 5, 6, 7, 8, 9, 10, 20, 30, 40 or 50 fold higher than the solubility of its corresponding polypeptide lacking the scaffold polypeptides, measured in the same solvent. In some embodiments, a solubility of the fusion polypeptide in an aqueous formulation is at least 1.1, 2, 3, 4, 5, 6, 7, 8, 9, 10, 20, 30, 40 or 50 fold higher than the solubility of its corresponding polypeptide lacking the scaffold polypeptides. The described fusion polypeptide can have an improved stability, e.g., serum stability, compared to the corresponding polypeptide lacking the scaffold domains. In some embodiments, a serum half-life of the fusion polypeptide is at least 1.1, 1.2, 1.3, 1.4, 1.5, 1.6, 1.7, 1.8, 1.9, 2, 3, 4, 5, 6, 7, 8, 9, or 10 fold longer than a serum half-life of its corresponding polypeptide lacking the scaffold polypeptides. In some embodiments, a serum half-life of the fusion polypeptide is at least 1.1, 2, 3, 4, 5, 6, 7, 8, 9, or 10 fold longer than a serum half-life of its corresponding polypeptide lacking the scaffold polypeptides. The described fusion polypeptide can have an improved solubility compared to the corresponding polypeptide lacking the scaffold domains. In some embodiments, a serum solubility of the fusion polypeptide is at least 1.1, 1.2, 1.3, 1.4, 1.5, 1.6, 1.7, 1.8, 1.9, 2, 3, 4, 5, 6, 7, 8, 9, or 10 fold higher than a serum solubility of its corresponding polypeptide lacking the scaffold polypeptides.

The described fusion polypeptide can elicit an enhanced immune response compared to the corresponding polypeptide lacking the scaffold domains. In some embodiments, an antigen-specific T cell response of the fusion polypeptide following administration to a subject is at least 1.1, 1.2, 1.3, 1.4, 1.5, 1.6, 1.7, 1.8, 1.9, 2, 3, 4, 5, 6, 7, 8, 9, 10, 20, 30, 40, 50, 100, 200, or 300 fold higher than an antigen-specific T cell response of its corresponding polypeptide lacking the scaffold polypeptides as measured by an increased splenocytes proliferation. For example, the antigen-specific T cell response can be measured according to the method in Example 3.2.

### Scaffold Polypeptides or Scaffold Domain

The fusion polypeptide can comprise one or more scaffold polypeptides sequences. In some embodiments, the one or more scaffold polypeptides are the same. In some embodiments, the one or more scaffold polypeptides comprise at least two distinct scaffold polypeptide sequences. In certain embodiments, the one or more scaffold polypeptides comprise 2, 3, 4, 5, 6, 7, 8, 9, 10, or more distinct scaffold polypeptide sequences.

In some embodiments, the one or more scaffold polypeptides comprise recombinant human polypeptides. In some embodiments, the one or more scaffold polypeptides comprise a sequence derived from a human protein. In some embodiments, the one or more scaffold polypeptides comprise a sequence having at least 80%, 85%, 90%, 95%, 99%, or 100% sequence identity to a wild type mammalian protein such as a human protein. In some embodiments, the one or more scaffold polypeptides are not configured to have targeted binding characteristics. In other embodiments, at least one of the scaffold polypeptides is configured to bind to a target molecule. In some embodiments, the scaffold polypeptides lack post-translational modifications. In some embodiments, the scaffold polypeptides lack intra-peptide disulfide bonds. In some embodiments, the one or more scaffold polypeptides are non-immunogenic. In other embodiments, at least one of the scaffold polypeptides is immunogenic. In some embodiments, the scaffold polypeptides are not configured to have enzymatic activity. In some embodiments, the one or more scaffold polypeptides lack a signal sequence.

In some embodiments, the fusion polypeptide comprises at least 2, 3, 4, 5, 6, 7, 8, 9, 10, 11, 12, 13, 14, or 15 scaffold polypeptides. In some embodiments, the fusion polypeptide comprises at most 5, 6, 7, 8, 9, 10, 11, 12, 13, 14, 15, or 20 scaffold polypeptides. In some embodiments, the fusion polypeptide comprises from 2 to 6 scaffold polypeptides. In some embodiments, the fusion polypeptide comprises 1, 2, 3, 4, 5, 6, 7, 8, 9, or 10 scaffold polypeptides. In certain specific embodiments, the fusion polypeptide comprises 6 scaffold polypeptides.

In some embodiments, a molecular mass of the scaffold polypeptides sequences in the fusion polypeptide is at least 11 kDa, 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, or 200 kDa. In some embodiments, a molecular mass of the scaffold polypeptides sequences in the fusion polypeptide is at least 75 kDa. In some embodiments, a molecular mass of the scaffold polypeptides sequences in the fusion polypeptide is at most 15 kDa, 20 kDa, 30 kDa, 35 kDa, 40 kDa, 45 kDa, 50 kDa, 55 kDa, 60 kDa, 65 kDa, 70 kDa, 75 kDa, 80 kDa, 85 kDa, 90 kDa, 100 kDa, 110 kDa, 120 kDa, 150 kDa, 200 kDa, or 500 kDa. In some embodiments, a molecular mass of each of the scaffold polypeptides sequences is at least 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 30 kDa, 40 kDa, 50 kDa, 60 kDa, 70 kDa, or 100 kDa. In some embodiments, a molecular mass of each of the scaffold polypeptides sequences is at most 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, 19 kDa, 20 kDa, 30 kDa, 40 kDa, 50 kDa, 60 kDa, 70 kDa, or 250 kDa. In some embodiments, at least one of the scaffold polypeptides has a molecular mass of from 8 to 12 kDa or 10 to 12 kDa. In some embodiments, at least one of the scaffold polypeptides has a molecular mass of about 11 kDa.

In some embodiments, each of the scaffold polypeptides sequences comprises at least 21, at least 30, at least 35, at least 40, at least 45, at least 50, at least 55, at least 60, at least 65, at least 70, at least 75, at least 80, at least 85, at least 90, at least 100, at least 125, at least 150, at least 175, or at least 200 amino acid residues. In some embodiments, each of the scaffold polypeptides sequences comprises at most 30, at most 35, at most 40, at most 45, at most 50, at most 55, at most 60, at most 65, at most 70, at most 75, at most 80, at most 85, at most 90, at most 100, at most 125, at most 150, at most 175, or at most 200 amino acid residues. In some embodiments, at least one of the scaffold polypeptides has at least 80, at least 85, at least 90, or at least 95 amino acid residues. In some embodiments, at least one of the scaffold polypeptides has about 90, 91, 92, 93, 94, 95, 96, 97, 98, 99, 100, 101, 102, 110, or 120 amino acid sequences.

In some embodiments, a scaffold protein is a mammalian protein, e.g., a human protein which can be suitably expressed in a bacterial expression system, such as E. coli expression system, wherein the protein is compact, stable, and is either a secreted protein or a cytoplasmic protein. In some embodiments, the scaffold protein is a human protein. Mammalian cells contain an estimated one billion protein molecules. A large fraction of these proteins, can be found to function as scaffolding proteins that help increase efficiency and support in biomolecular interactions inside the body, where the interactions promote an aspect of cellular behavior, such as exerting signal transduction, intracellular trafficking of biomolecules, enzymatic activity or structural reshaping promoting cellular motility. In the present disclosure we provide an efficient and elegant way of using human scaffold proteins to turn short epitope peptides into means for generating active immune response. In some embodiments, the scaffold proteins are selected so as to impart stability to the epitope peptides. In some embodiments the scaffold proteins are selected to impart solubility and increased serum half-life to the epitope peptides. In some embodiments the scaffold proteins are selected to impart structural advantage in rendering immunogenicity, for example by increasing availability for association with a cognate MHC molecule. The scaffold protein may be selected so that it is non-toxic. The scaffold proteins can be any protein having a scaffolding function or ability to suit any or all of these objectives.

For example, known cellular scaffold proteins assist other functional proteins to reduce entropy of a reaction. In some embodiments, a scaffold protein helps tether one or more proteins, enzymes, peptides, or other biomolecules, increase the local concentration or immobilize temporarily for a functional outcome. For example, Cullin scaffold proteins tether E2 ubiquitin; the linker for activation of T cells (LAT) and SH2-domain containing leukocyte protein of 76 kD (SLP-76) proteins help organize TCR signaling.

In some embodiments, at least one of the one or more scaffold polypeptides is a member of the cystatin superfamily, a variant thereof, or a fragment thereof. In some embodiments, the member of the cystatin superfamily is a type 1 cystatin, type 2 cystatin, or type 3 cystatin. In some embodiments, at least one of the one or more scaffold polypeptides is Stefin A such as human Stefin A. In some embodiments, all of the scaffold polypeptides are Stefin A. In some embodiments, the one or more scaffold polypeptides comprise a sequence having at least 80%, 85%, 90%, 95%, 99%, or 100% sequence identity to a human Stefin A protein sequence.

In some embodiments, the one or more scaffold polypeptides comprise a sequence of a mammalian titin protein, a variant thereof, or a fragment thereof. The sequence of the titin protein can be a sequence from any domain of the titin protein, e.g., the Ig-like domains, the FnIII-like domains, and the Pseudokinase domain. In some embodiments, the sequence of the titin protein is a sequence of the I-band. In some embodiments, the one or more scaffold polypeptides comprise a sequence having at least 80%, 85%, 90%, 95%, 99%, or 100% sequence identity to a titin Ig-like domain sequence. In some embodiments, the sequence from the titin protein is an 127 domain sequence, an I1 domain sequence, a Z1 domain sequence, a Z2 domain sequence, or a M5 domain sequence. In some embodiments, the sequence from the titin protein is an 127 domain sequence. In some embodiments, the one or more scaffold polypeptides comprise titin 127, a variant thereof, or a fragment thereof.

In some embodiments, the one or more scaffold polypeptides comprise a sequence of a glycoprotein, such as fibronectin, a variant thereof, or a fragment thereof. In some embodiments, the glycoprotein is fibronectin. The sequence of fibronectin can be a sequence from any of its subunits, e.g., fibronectin type I, type II, or type II domain. In some embodiments, the one or more scaffold polypeptides comprise a sequence having at least 80%, 85%, 90%, 95%, 99%, or 100% sequence identity to a sequence of FnIII, e.g., 1FNIII, 9FNIII, 10FNIII, or 11FNIII. In some embodiments, the sequence of FNIII is 10FN-III domain sequence.

In some embodiments, the one or more scaffold polypeptides comprise a polypeptide sequence selected from: thioredoxin, 10FNIII, lipocalins, capsid polypeptides of adeno-associated viruses, alpha-amylase inhibitors, Stefin A, ubiquitin, Ig-L filamin A, tenascin, inactivated staphylococcal nuclease, green fluorescent protein, isolated protein folds such as the Z domain of staphylococcal protein A, ankyrin repeats, bilin binding protein, a fragment thereof, and a variant thereof. In some embodiments, the one or more scaffold polypeptides are each independently selected from Titin 127, ubiquitin, Stefin A, 10FN-III, Ig-L filamin A, tenascin, a fragment thereof, and a variant thereof. In some embodiments, the scaffold polypeptides are each independently selected from Stefin A, Titin I27, a fragment thereof, and a variant thereof. In some embodiments, each of the scaffold polypeptides is Stefin A, a fragment thereof, or a variant thereof.

A scaffold protein can be full length proteins or fragments or specific domains. In some embodiments, a scaffold can be a Protein A domain, a Src homology domain, a PDZ domain, a WW domain, a zinc finger domain, or derivatives thereof.

In some embodiments, the scaffold protein may be modified to suit the need. In some embodiment, the scaffold is mutated at 1, 2, 3, or more residues to reduce toxicity, to reduce biological interaction, or to reduce immunogenicity. In some embodiment, the scaffold is mutated at 1, 2, 3, or more residues to include a binding site or a specificity determinant moiety, for example a targeting moiety, such as incorporating dendritic cell (DC) targeting CLEC9A moiety.

### Antigen Polypeptide

The fusion polypeptide described herein can comprise one or more antigen polypeptides. In some embodiments, the fusion polypeptide comprises at least 2, 3, 4, 5, 6, 7, 8, 9, 10, or 20 antigen polypeptides. In some embodiments, the fusion polypeptide comprises at most 2, 3, 4, 5, 6, 7, 8, 9, 10, 20, 50, or 100 antigen polypeptides. In some embodiments, the fusion polypeptide comprises 1, 2, 3, 4, 5, or 6 antigen polypeptides. In some embodiments, the fusion polypeptide comprises 5 or 6 antigen polypeptides.

In some embodiments, the fusion polypeptide comprises a plurality of antigen polypeptides. In some embodiments, each of the antigen polypeptide sequences is distinct from other antigen polypeptide sequences on the fusion polypeptide. In some embodiments, all of the antigen polypeptides of a fusion polypeptide have the same sequence. In some embodiments, at least one of the antigen polypeptides is a synthetic polypeptide.

The one or more antigen polypeptides can comprise any antigen polypeptide that induces an immune response, e.g., an exogenous antigen, an endogenous antigen, an autoantigen, a neoantigen, or a combination thereof. In some embodiments, at least one antigen binds to a class I HLA protein to form a class I HLA-peptide complex. In some embodiments, at least one antigen binds to a class II HLA a protein to form a class II HLA-peptide complex. In some embodiments, at least one antigen activates CD8⁺ T cells. In some embodiments, at least one antigen activates CD4⁺ T cells. In some embodiments, the one or more antigen polypeptides comprise a first antigen polypeptide and a second antigen polypeptide. In some embodiments, the first antigen polypeptide binds to a class I HLA protein to form a class I HLA-peptide complex. In some embodiments, the second antigen polypeptide binds to a class II HLA a protein to form a class II HLA-peptide complex. In some embodiments, the second antigen polypeptide binds to a class I HLA protein to form a class I HLA-peptide complex. In some embodiments, the first antigen polypeptide binds to a class II HLA protein to form a class II HLA-peptide complex. In some embodiments, a single polypeptide may comprise a class I HLA binding epitope and a class II HLA binding epitope. In some embodiments, the first antigen polypeptide activates CD8⁺ T cells. In some embodiments, the second antigen polypeptide activates CD4⁺ T cells. In some embodiments, a linker sequence is directly connected to a scaffold polypeptide sequence and an antigen polypeptide sequence. In some embodiments, a linker sequence is directly connected to a scaffold polypeptide sequence and another linker sequence. In some embodiments, a linker sequence is directly connected to an antigen polypeptide sequence and another linker sequence. In some embodiments, a linker sequence is directly connected to two scaffold polypeptide sequences. In some embodiments, a linker sequence is directly connected to two antigen polypeptide sequences. In some embodiments, a linker sequence is directly connected to two other linker sequences. In some embodiments, a linker sequence can be directly connected to three or more of the following: scaffold polypeptide sequence(s), antigen polypeptide sequence(s), linker sequence(s), or any combination thereof.

In some embodiments, the one or more linker sequences are the same. In some embodiments, each of the one or more linker sequences is unique. In some embodiments, the fusion polypeptide comprises at least two distinct linker sequences. In some embodiments, each of the linker sequences comprises at least 2, 3, 4, 5, 6, 7, 8, 9, 10, 15, 20, 25, or 50 amino acid residues. In some embodiments, each of the linker sequences comprises at most 2, 3, 4, 5, 6, 7, 8, 9, 10, 15, 20, 25, 50, or 100 amino acid residues. In some embodiments, one or more linker sequences comprise from 5 to 20 amino acid residues. In some embodiments, the linker sequences are non-immunogenic. In some embodiments, the linker sequences are not configured to form a disulfide bond. In some embodiments, a linker comprises 1 to 100 amino acid residues, 2 to 50 amino acid residues, or 2 to 25 amino acid residues. In some embodiments, the linkers are flexible. In some embodiments, at least one of the linkers is flexible. In some embodiments, a flexible linker comprises small, non-polar amino acids, e.g., Gly, Ser, and Thr. In some embodiments, a flexible linker comprises stretches of Gly and Ser residues, e.g., Gly-Gly-Gly-Gly-Ser. In some embodiments, a flexible linker comprises a sequence of KESGSVSSEQLAQFRSLD. In some embodiments, a flexible linker comprises a sequence of EGKSSGSGSESKST. In some embodiments, a flexible linker comprises a sequence of (Gly)z, and z is an integer from 5 to 10. In some embodiments, a flexible linker comprises a sequence of (Gly)₈. In some embodiments, a flexible linker comprises a sequence of GSAGSAAGSGEF. In some embodiments, the linkers are rigid. In some embodiments, at least one of the linkers is rigid. In some embodiments, a rigid linker can form an alpha helix. In some embodiments, a rigid linker comprises a sequence of EAAAK. In some embodiments, a rigid linker comprises a sequence of Glu-Pro. In some embodiments, a rigid linker comprises a sequence of Lys-Pro. Exemplary linker sequences can include the ones disclosed in Chen, et al, Adv Drug Deliv Rev. 2013 Oct. 15; 65(10): 1357-1369, “Fusion Protein Linkers: Property, Design and Functionality.”

In some embodiments, the fusion polypeptide is configured to facilitate a cleavage of the linker. In some embodiments, the fusion polypeptide is configured to facilitate a cleavage of at least one of the one or more antigen polypeptides. In some embodiments, the linkers are cleavable. In some embodiments, at least one of the linkers is cleavable. A cleavable linker can comprise one or more cleavable sites. In some embodiments, at least one of the linker sequences comprises a cleavage site. In some embodiments, the cleavage site is cleavable by a cellular reducing agent. For example, the cleavable site can comprise a disulfide bond, e.g., a disulfide bond formed between two cysteine residues on the linker. In some embodiments, the cleavage site is cleavable by a peptidase or a protease. In some embodiments, the cleavage site is cleavable by a protease, e.g., Kex1, Ste13, and Kex2.

In some embodiments, the fusion polypeptide is configured to facilitate presentation of at least one of the one or more antigen polypeptides. In some embodiments, the linkers comprise a context sequence that can facilitate the presentation of the antigen polypeptides. In some embodiments, at least one of the linker sequences comprises a lysine residue, an arginine residue, a serine residue, an asparagine residue, a histidine residue, an alanine residue, a glutamine residue, an aspartic acid residue, a methionine residue, a tyrosine residue, or any combination thereof. In some embodiments, at least one of the linker sequences comprises a lysine residue, an arginine residue, or an alanine residue directly connected to the N-terminus of at least one of the antigen polypeptides. In some embodiments, at least one of the linker sequences comprises a serine residue, a lysine residue, an arginine residue, or an alanine residue directly connected to the C-terminus of at least one of the antigen polypeptides. In some embodiments, at least one of the linker sequences comprises an aspartic acid residue, a methionine residue, a leucine residue, a valine residue, an isoleucine residue, a tyrosine residue, a phenylalanine residue, a tryptophan residue, or any combination thereof. In some embodiments, at least one of the linker sequences comprises an aspartic acid residue, a methionine residue, a leucine residue, a tyrosine residue, or a phenylalanine residue directly connected to the N-terminus of at least one of the antigen polypeptides. In some embodiments, at least one of the linker sequences comprises a methionine residue, a leucine residue, a valine residue, an isoleucine residue, a tyrosine residue, a phenylalanine residue, or a tryptophan residue directly connected to the C-terminus of at least one of the antigen polypeptides.

In some embodiments, at least one of the linkers is functional. For example, a linker can be configured to improve the expression level of the fusion polypeptide, to improve the bioactivity of the fusion polypeptide, or to enable the fusion polypeptide to target specific sites in vivo. For another example, a linker can be configured to affect the PK and PD properties of the fusion polypeptide.

### Targeting Function

In some embodiments, the fusion polypeptide is configured to target certain sites such as an in vivo target site. For example, the fusion polypeptide can have one or more binding moieties that have high affinity or bind to the target site. Exemplary target sites can include, but are not limited to, an agent, a drug, a protein or polypeptide, and a cell such as an antigen presenting cell. In some embodiments, the fusion polypeptide comprises one or more binding moieties configured to bind to an antigen presenting cell, to an adjuvant, or to a reagent. In some embodiments, the antigen presenting cell is a dendritic cell (DC), a macrophage, a Langerhans cell, or a B cell.

In some embodiments, the one or more binding moieties are configured to bind to one or more receptors expressed on a cell, such as a dendritic cell.

In some embodiments, the one or more receptors comprise a Calcium-dependent (C-type) lectin receptor, a scavenger receptor, an F4/80 Receptor, a DC-specific transmembrane protein (DC-STAMP), an Fc receptor, or any combination thereof.

In some embodiments, the C-type lectin receptor is a mannose receptor, a dendritic cell-specific intercellular adhesion molecule-3-grabbing nonintegrin (DC-SIGN) receptor, an L-SIGN or DC-SIGNR receptor, a liver and lymph node sinusoidal cell type lectin (LSECtin) receptor, a C-type lectin immune receptor (CIRE), a langerin receptor, a human macrophage galactose- and N-acetylgalactosamine-specific C-type lectin receptor, a Dectin-1 or beta-glucan receptor, an NK lectin group receptor-1, a myeloid inhibitory C-type lectin receptor, a C-type lectin-like receptor 2 (CLEC2), a CLEC12B receptor, a lectin-like receptor for oxidized density lipoprotein-1, a DC immunoreceptor subfamily receptor, a DC immunoreceptor, a Dectin-2 receptor, or a Blood DC antigen.

In some embodiments, the one or more receptors comprise Clec9a.

In some embodiments, the one or more receptors comprise a chemokine receptor.

In some embodiments, the one or more receptors comprise XCR1 receptor.

In some embodiments, at least one of the binding moieties is comprised in the scaffold polypeptides.

In some embodiments, at least one of the binding moieties is directly connected to an N-terminus or a C-terminus of one of the scaffold polypeptides.

In some embodiments, at least one of the binding moieties is directly connected to an N-terminus or a C-terminus of one of the antigen polypeptides.

In some embodiments, in the N-terminus to C-terminus direction, at least one of the binding moieties is terminally linked to a first or a last scaffold polypeptide of the scaffold polypeptides.

In some embodiments, the nucleic acid molecule encodes at least 2, 3, 4, 5, 6, 7, 8, 9, 10, or 20 different fusion polypeptides.

In some embodiments, the nucleic acid molecule encodes at most 2, 3, 4, 5, 6, 7, 8, 9, 10, or 20 different fusion polypeptides.

In some embodiments, the nucleic acid molecule is RNA or DNA.

In one aspect, disclosed herein is a fusion polypeptide encoded by the described nucleic acid molecule.

In one aspect, disclosed herein is a composition comprising the described fusion polypeptide. In some embodiments, the composition comprises one or more binding moieties capable of conjugating with the fusion polypeptide. In some embodiments, the one or more binding moieties are conjugated with the fusion polypeptide.

In one aspect, disclosed herein is a nucleic acid molecule encoding: two or more scaffold polypeptides interspaced by one or more linkers; and one or more restriction sites located on at least one of the linkers, wherein (i) a molecular mass of the two or more scaffold polypeptide sequences is more than 11 kDa or (ii) each of the two or more scaffold polypeptides sequences comprises at least 21 amino acid residues.

In one aspect, disclosed herein is a plurality of nucleic acid molecules comprising: a first nucleic acid molecule comprising, a nucleic acid sequence encoding a first antigen polypeptide, and a nucleic acid sequence encoding a first scaffold polypeptide; and a second nucleic acid molecule comprising, a nucleic acid sequence comprising a sequence complementary to the nucleic acid sequence encoding a first antigen polypeptide, and a nucleic acid sequence encoding a second scaffold polypeptide.

In another aspect, disclosed herein is a plurality of nucleic acid molecules comprising: a first nucleic acid molecule comprising, a nucleic acid sequence encoding a first antigen polypeptide, a nucleic acid sequence encoding a first scaffold polypeptide, and a nucleic acid sequence encoding a second antigen polypeptide; and a second nucleic acid molecule comprising, a nucleic acid sequence comprising a sequence complementary to the nucleic acid sequence encoding a second antigen polypeptide, and a nucleic acid sequence encoding a second scaffold polypeptide.

In yet another aspect, disclosed herein is a plurality of nucleic acid molecules comprising: a first nucleic acid molecule comprising, a nucleic acid sequence encoding a first antigen polypeptide, a nucleic acid sequence encoding a first scaffold polypeptide, and a nucleic acid sequence encoding a second antigen polypeptide; and a second nucleic acid molecule comprising a nucleic acid sequence comprising a sequence complementary to the nucleic acid sequence encoding a second antigen polypeptide, a nucleic acid sequence encoding a second scaffold polypeptide, and a nucleic acid sequence encoding a third antigen polypeptide.

In some embodiments, the first and the second scaffold sequences are the same.

In some embodiments, the first antigen polypeptide and the second antigen polypeptides are different.

In some embodiments, the first, the second, and the third antigen polypeptides are different.

In some embodiments, the nucleic acid molecules are RNA or DNA.

In one aspect, disclosed herein is a pharmaceutical composition comprising: a pharmaceutically acceptable excipient, carrier, or diluent, and the described composition or the described fusion polypeptide.

In some embodiments, the pharmaceutical composition comprises an adjuvant.

In some embodiments, the adjuvant is polyIC:LC.

In some embodiments, the pharmaceutical composition comprises a pH modifier.

In some embodiments, the pharmaceutical composition comprises a second therapeutic agent, such as an immunomodulator, a cytokine, a chemokine, or a check point inhibitor.

In one aspect, disclosed herein is a method of producing an immunogenic fusion polypeptide, the method comprising, expressing the described nucleic acid molecule in a genetically modified cell, thereby producing the immunogenic fusion polypeptide.

In another aspect, disclosed herein is a method of producing an immunogenic fusion polypeptide, the method comprising: providing a nucleic acid molecule as described herein; inserting one or more nucleic acid molecules encoding one or more antigen polypeptides to at least one of the restriction sites, thereby producing a new nucleic acid molecule; and expressing the new nucleic acid molecule in a genetically modified cell, thereby producing the immunogenic fusion polypeptide.

In some embodiments, the one or more nucleic acid molecules encoding one or more antigen polypeptides are inserted through an isothermal reaction or by restriction enzyme based cloning.

In one aspect, disclosed herein is a method of producing an immunogenic fusion polypeptide, the method comprising: providing a plurality of nucleic acid molecules as described herein; joining the nucleic acid molecules through hybridization; and expressing the joined nucleic acid molecules in a genetically modified cell, thereby producing the immunogenic fusion polypeptide.

In some embodiments, the fusion polypeptide is expressed in a bacterial expression system.

In some embodiments, the bacterial expression system is an Escherichia coli expression system.

In one aspect, disclosed herein is a method of treating or preventing cancer in a human subject in need thereof comprising, administering to the subject in need thereof the pharmaceutical composition as described herein.

In some embodiments, the pharmaceutical composition comprises a plurality of neoantigen peptides.

In some embodiments, the pharmaceutical composition comprises a plurality of fusion polypeptides.

In some embodiments, the pharmaceutical composition is administered intravenously or subcutaneously.

In some embodiments, a dose of the fusion polypeptide is divided into at least 2, at least 3, at least 4 or at least 5 sub-doses.

In some embodiments, each sub-dose of the fusion polypeptide comprises 1, 2, 3, 4, 5, or more fusion polypeptides.

In some embodiments, each fusion polypeptide is administered at a dose of from 0.01-100 g.

In some embodiments, each fusion polypeptide is administered at a dose of from 100 □g-10 mg.

In some embodiments, a total dose of the fusion polypeptides administered is from 0.1-100 mg.

### Neoantigens and Uses Thereof

One of the critical barriers to developing curative and tumor-specific immunotherapy is the identification and selection of highly specific and restricted tumor antigens to avoid autoimmunity. Tumor neoantigens, which arise as a result of genetic change (e.g., inversions, translocations, deletions, missense mutations, splice site mutations, etc.) within malignant cells, represent the most tumor-specific class of antigens. Neoantigens have rarely been used in cancer vaccine or immunogenic compositions due to technical difficulties in identifying them, selecting optimized antigens, and producing neoantigens for use in a vaccine or immunogenic composition. These problems may be addressed by: identifying mutations in neoplasias/tumors which are present at the DNA level in tumor but not in matched germline samples from a high proportion of subjects having cancer; analyzing the identified mutations with one or more peptide-MHC binding prediction algorithms to generate a plurality of neoantigen T cell epitopes that are expressed within the neoplasia/tumor and that bind to a high proportion of patient HLA alleles; and synthesizing the plurality of neoantigenic peptides selected from the sets of all neoantigen peptides and predicted binding peptides for use in a cancer vaccine or immunogenic composition suitable for treating a high proportion of subjects having cancer.

For example, translating peptide sequencing information into a therapeutic vaccine may include prediction of mutated peptides that can bind to HLA molecules of a high proportion of individuals. Efficiently choosing which particular mutations to utilize as immunogen requires the ability to predict which mutated peptides would efficiently bind to a high proportion of patient's HLA alleles. Recently, neural network based learning approaches with validated binding and non-binding peptides have advanced the accuracy of prediction algorithms for the major HLA-A and -B alleles. However, even using advanced neural network-based algorithms to encode HLA-peptide binding rules, several factors limit the power to predict peptides presented on HLA alleles.

Another example of translating peptide sequencing information into a therapeutic vaccine may include formulating the drug as a multi-epitope vaccine of long peptides. Targeting as many mutated epitopes as practically possible takes advantage of the enormous capacity of the immune system, prevents the opportunity for immunological escape by down-modulation of an immune targeted gene product, and compensates for the known inaccuracy of epitope prediction approaches. Provided herein is a polypeptide comprising multiple epitopes, for generating a therapeutic product, or a vaccine, wherein the polypeptide is expressed from a polynucleotide in a host cell or by in vitro translation. In some embodiments, the polypeptide can be manufactured synthetically. As described, the polypeptide comprising multiple epitopes can further comprise one or more scaffold proteins. In some embodiments, provided herein are synthetic polybodies comprising multiple epitopes, scaffold proteins and linkers, for use as a therapeutic vaccine.

Yet another example of translating peptide sequencing information into a therapeutic vaccine may include a combination with a strong vaccine adjuvant. Effective vaccines may require a strong adjuvant to initiate an immune response. For example, poly-ICLC, an agonist of TLR3 and the RNA helicase-domains of MDA5 and RIG3, has shown several desirable properties for a vaccine adjuvant. These properties include the induction of local and systemic activation of immune cells in vivo, production of stimulatory chemokines and cytokines, and stimulation of antigen-presentation by DCs. Furthermore, poly-ICLC can induce durable CD4⁺ and CD8⁺ responses in humans. Importantly, striking similarities in the upregulation of transcriptional and signal transduction pathways were seen in subjects vaccinated with poly-ICLC and in volunteers who had received the highly effective, replication-competent yellow fever vaccine. Furthermore, >90% of ovarian carcinoma patients immunized with poly-ICLC in combination with a NYESO-1 peptide vaccine (in addition to Montanide) showed induction of CD4⁺ and CD8⁺ T cell, as well as antibody responses to the peptide in a recent phase 1 study. At the same time, poly-ICLC has been extensively tested in more than 25 clinical trials to date and exhibited a relatively benign toxicity profile.

In some aspects, provided herein is a composition comprising: a first peptide comprising a first neoepitope of a protein and a second peptide comprising a second neoepitope of the same protein, a polynucleotide encoding the first peptide and the second peptide, one or more APCs comprising the first peptide and the second peptide, or a first T cell receptor (TCR) specific for the first neoepitope in complex with an HLA protein and a second TCR specific for the second neoepitope in complex with an HLA protein; wherein the first peptide is different from the second peptide, and wherein the first neoepitope comprises a mutation and the second neoepitope comprises the same mutation.

In some aspects, provided herein is a composition comprising: a first peptide comprising a first neoepitope of a region of a protein and a second peptide comprising a second neoepitope of the region of the same protein, wherein the first neoepitope and the second neoepitope comprise at least one amino acid of the region that is the same, a polynucleotide encoding the first peptide and the second peptide, on or more APCs comprising the first peptide and the second peptide, or a first T cell receptor (TCR) specific for the first neoepitope in complex with an HLA protein and a second TCR specific for the second neoepitope in complex with an HLA protein; wherein the first peptide is different from the second peptide, and wherein the first neoepitope comprises a first mutation and the second neoepitope comprises a second mutation.

In some embodiments, the first mutation and the second mutation are the same. In some embodiments, the first peptide and the second peptide are different molecules. In some embodiments, the first neoepitope comprises a first neoepitope of a region of the same protein, wherein the second neoepitope comprises a second neoepitope of the region of the same protein. In some embodiments, the first neoepitope and the second neoepitope comprise at least one amino acid of the region that is the same. In some embodiments, the region of the protein comprises at least 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, 600, 700, 800, 900, or 1,000 contiguous amino acids of the protein. In some embodiments, the region of the protein comprises at most 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, 500, 600, 700, 800, 900, or 1,000 contiguous amino acids of the protein. In some embodiments, the first neoepitope binds to a class I HLA protein to form a class I HLA-peptide complex. In some embodiments, the second neoepitope binds to a class II HLA protein to form a class II HLA-peptide complex. In some embodiments, the second neoepitope binds to a class I HLA protein to form a class I HLA-peptide complex. In some embodiments, the first neoepitope binds to a class II HLA protein to form a class II HLA-peptide complex. In some embodiments, the first neoepitope is a first neoepitope peptide processed from the first peptide and/or the second neoepitope is a second neoepitope peptide processed from the second peptide. In some embodiments, the first neoepitope is shorter in length than first peptide and/or the second neoepitope is shorter in length than second peptide. In some embodiments, the first neoepitope peptide is processed by an antigen presenting cell (APC) comprising the first peptide and/or the second neoepitope peptide is processed by an APC comprising the second peptide. In some embodiments, the first neoepitope activates CD8⁺ T cells. In some embodiments, the second neoepitope activates CD4⁺ T cells. In some embodiments, the second neoepitope activates CD8⁺ T cells. In some embodiments, the first neoepitope activates CD4⁺ T cells. In some embodiments, a TCR of a CD4⁺ T cell binds to a class II HLA-peptide complex comprising the first or second peptide. In some embodiments, a TCR of a CD8⁺ T cell binds to a class I HLA-peptide complex comprising the first or second peptide. In some embodiments, a TCR of a CD4⁺ T cell binds to a class I HLA-peptide complex comprising the first or second peptide. In some embodiments, a TCR of a CD8⁺ T cell binds to a class II HLA-peptide complex comprising the first or second peptide. In some embodiments, the one or more APCs comprise a first APC comprising the first peptide and a second APC comprising the second peptide. In some embodiments, the mutation is selected from the group consisting of a point mutation, a splice-site mutation, a frame shift mutation, a read-through mutation, a gene fusion mutation and any combination thereof.

In some embodiments, a single polypeptide comprises the first peptide and the second peptide, or a single polynucleotide encodes the first peptide and the second peptide. In some embodiments, the first peptide and the second peptide are encoded by a sequence transcribed from a same transcription start site. In some embodiments, the first peptide is encoded by a sequence transcribed from a first transcription start site and the second peptide is encoded by a sequence transcribed from a second transcription start site. In some embodiments, the single polypeptide has a length of at least 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the polypeptide comprises a first sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a first corresponding wild-type sequence; and a second sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a corresponding second wild-type sequence. In some embodiments, the polypeptide comprises a first sequence of at least 8 or 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a corresponding first wild-type sequence; and a second sequence of at least 16 or 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, sequence identity to a corresponding second wild-type sequence. In some embodiments, the second peptide is longer than the first peptide. In some embodiments, the first peptide is longer than the second peptide. In some embodiments, the first peptide has a length of at least 9; 10; 11; 12; 13; 14; 15; 16; 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the second peptide has a length of at least 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the first peptide comprises a sequence of at least 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a corresponding wild-type sequence. In some embodiments, the second peptide comprises a sequence of at least 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a corresponding wild-type sequence. In some embodiments, the second neoepitope is longer than the first neoepitope. In some embodiments, the first neoepitope has a length of at least 8 amino acids. In some embodiments, the first neoepitope has a length of from 8 to 12 amino acids. In some embodiments, the first neoepitope comprises a sequence of at least 8 contiguous amino acids, wherein at least 2 of the 8 contiguous amino acids are different at corresponding positions of a wild-type sequence. In some embodiments, the second neoepitope has a length of at least 16 amino acids. In some embodiments, the second neoepitope has a length of from 16 to 25 amino acids. In some embodiments, the second neoepitope comprises a sequence of at least 16 contiguous amino acids, wherein at least 2 of the 16 contiguous amino acids are different at corresponding positions of a wild-type sequence.

In some embodiments, the first peptide comprises at least one additional mutation. In some embodiments, one or more of the at least one additional mutation is not a mutation in the first neoepitope. In some embodiments, one or more of the at least one additional mutation is a mutation in the first neoepitope. In some embodiments, the second peptide comprises at least one additional mutation. In some embodiments, one or more of the at least one additional mutation is not a mutation in the second neoepitope. In some embodiments, one or more of the at least one additional mutation is a mutation in the second neoepitope. In some embodiments, the first peptide, the second peptide or both comprise at least one flanking sequence, wherein the at least one flanking sequence is upstream or downstream of the neoepitope. In some embodiments, the at least one flanking sequence has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a corresponding wild-type sequence. In some embodiments, the at least one flanking sequence comprises a non-wild-type sequence. In some embodiments, the at least one flanking sequence is a N-terminus flanking sequence. In some embodiments, the at least one flanking sequence is a C-terminus flanking sequence. In some embodiments, the at least one flanking sequence of the first peptide has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the at least one flanking sequence of the second peptide. In some embodiments, the at least one flanking region of the first peptide is different from the at least one flanking region of the second peptide. In some embodiments, the at least one flanking residue comprises the mutation.

In some embodiments, the composition comprises one or more additional peptides, wherein the one or more additional peptides comprise a third neoepitope. In some embodiments, the first and/or second neoepitope binds to an HLA protein with a greater affinity than a corresponding wild-type sequence. In some embodiments, the first and/or second neoepitope binds to an HLA protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class I protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class II protein with a K_{D} or an IC₅₀ less than 1000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to a protein encoded by an HLA allele expressed by a subject. In some embodiments, the mutation is not present in non-cancer cells of a subject. In some embodiments, the first and/or second neoepitope is encoded by a gene or an expressed gene of a subject's cancer cells.

In some embodiments, the composition comprising polybodies having multiple epitopes are capable of activating a first T cell comprising a first TCR. In some embodiments, the composition having multiple epitopes are capable of activating a second T cell comprising a second TCR. In some embodiments, the first and/or second T cell is a cytotoxic T cell. In some embodiments, the first and/or second T cell is a gamma delta T cell. In some embodiments, the first and/or second T cell is a helper T cell. In some embodiments, the first T cell is a T cell stimulated, expanded or induced with the first neoepitope and/or the second T cell is a T cell stimulated, expanded or induced with the second neoepitope.

In some embodiments, the first and/or second TCR binds to an HLA-peptide complex with a K_{D} or an IC₅₀ of less than 1000 nM, 900 nM, 800 nM, 700 nM, 600 nM, 500 nM, 250 nM, 150 nM, 100 nM, 50 nM, 25 nM or 10 nM. In some aspects, provided herein is a vector comprising a polynucleotide encoding a first and a second peptide described herein. In some embodiments, the polynucleotide is operably linked to a promoter. In some embodiments, the vector is a self-amplifying RNA replicon, plasmid, phage, transposon, cosmid, virus, or virion. In some embodiments, the vector is a viral vector. In some embodiments, the vector is derived from a retrovirus, lentivirus, adenovirus, adeno-associated virus, herpes virus, pox virus, alpha virus, vaccina virus, hepatitis B virus, human papillomavirus or a pseudotype thereof. In some embodiments, the vector is a non-viral vector. In some embodiments, the non-viral vector is a nanoparticle, a cationic lipid, a cationic polymer, a metallic nanopolymer, a nanorod, a liposome, a micelle, a microbubble, a cell-penetrating peptide, or a liposphere.

In some aspects, provided herein is a pharmaceutical composition comprising: a composition described herein, or a vector described herein; and a pharmaceutically acceptable excipient.

In some embodiments, the plurality of cells is autologous cells. In some embodiments, the plurality of APC cells is autologous cells. In some embodiments, the plurality of T cells is autologous cells. In some embodiments, the pharmaceutical composition further comprises an immunomodulatory agent or an adjuvant. In some embodiments, the immunomodulatory agent is a cytokine. In some embodiments, the adjuvant is Hiltonol.

In some aspects, provided herein is a method of treating cancer, the method comprising administering to a subject in need thereof a pharmaceutical composition described herein.

In some aspects, provided herein is a method of preventing resistance to a cancer therapy, the method comprising administering to a subject in need thereof a pharmaceutical composition described herein.

In some aspects, provided herein is a method of inducing an immune response, the method comprising administering to a subject in need thereof a pharmaceutical composition described herein.

In some embodiments, the immune response is a humoral response.

In some embodiments, provided herein are polypeptides comprising one or more epitope peptides, and scaffold proteins that together form polybodies. In some embodiments, the polybodies are homopolymers. In some embodiments, the polybodies comprise a plurality of a first peptide. In some embodiments, the polybodies comprise a plurality of a second peptide. In some embodiments, the polybodies comprise a plurality of a third peptide. In some embodiments, the first peptide and the second peptide are administered simultaneously, separately or sequentially. In some embodiments, the first peptide is sequentially administered after the second peptide. In some embodiments, the second peptide is sequentially administered after the first peptide. In some embodiments, the first peptide is sequentially administered after a time period sufficient for the second peptide to activate the T cells. In some embodiments, the second peptide is sequentially administered after a time period sufficient for the first peptide to activate the T cells. In some embodiments, the first peptide is sequentially administered after the second peptide to restimulate the T cells. In some embodiments, the second peptide is sequentially administered after the first peptide to restimulate the T cells. In some embodiments, the first peptide is administered to stimulate the T cells and the second peptide is administered after the first peptide to restimulate the T cells. In some embodiments, the second peptide is administered to stimulate the T cells and the first peptide is administered after the second peptide to restimulate the T cells.

In some embodiments provided herein are polypeptides comprising one or more epitope peptides, and scaffold proteins that together form polybodies. In some embodiments, the polybodies are heteropolymers. In some embodiments, the polybodies comprise a plurality of a first peptide, and a plurality of a second peptide, and/or a third peptide, and/or a forth peptide, and/or a fifth peptide, and/or a sixth peptide, and/or more than six peptides. In some embodiments, the polybodies comprise a plurality of a third peptide. In some embodiments, the subject has cancer, wherein the cancer is selected from the group consisting of melanoma, ovarian cancer, lung cancer, prostate cancer, breast cancer, colorectal cancer, endometrial cancer, and chronic lymphocytic leukemia (CLL). In some embodiments, the subject has a breast cancer that is resistant to anti-estrogen therapy. In some embodiments, the breast cancer expresses an estrogen receptor with a mutation. In some embodiments, the subject has a CLL that is resistant to ibrutinib therapy. In some embodiments, the CLL expresses a Bruton tyrosine kinase with a mutation, such as a C481S mutation. In some embodiments, the subject has a lung cancer that is resistant to a tyrosine kinase inhibitor. In some embodiments, the lung cancer expresses an epidermal growth factor receptor (EGFR) with a mutation, such as a T790M, L792F, or C797S mutation. In some embodiments, the plurality of APC cells comprising the first peptide and the plurality of APC cells comprising the second peptide are administered simultaneously, separately or sequentially. In some embodiments, the method further comprises administering at least one additional therapeutic agent or modality. In some embodiments, the at least one additional therapeutic agent or modality is surgery, a checkpoint inhibitor, an antibody or fragment thereof, a chemotherapeutic agent, radiation, a vaccine, a small molecule, a T cell, a vector, and APC, a polynucleotide, an oncolytic virus or any combination thereof. In some embodiments, the at least one additional therapeutic agent is an anti-PD-1 agent and anti-PD-L1 agent, an anti-CTLA-4 agent, or an anti-CD40 agent. In some embodiments, the additional therapeutic agent is administered before, simultaneously, or after administering a pharmaceutical composition according described herein. In some embodiments, the additional therapeutic is a chemokine or a cytokine. Exemplary cytokines are interleukins, such as IL-1, IL-12, or a combination of more than two cytokines, more than three cytokines or more four cytokines and so on.

### Peptides

The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

In some embodiments, sequencing methods are used to identify tumor specific mutations. Any suitable sequencing method can be used according to the present disclosure, for example, Next Generation Sequencing (NGS) technologies. Third Generation Sequencing methods might substitute for the NGS technology in the future to speed up the sequencing step of the method. For clarification purposes: the terms “Next Generation Sequencing” or “NGS” in the context of the present disclosure mean all novel high throughput sequencing technologies which, in contrast to the “conventional” sequencing methodology known as Sanger chemistry, read nucleic acid templates randomly in parallel along the entire genome by breaking the entire genome into small pieces. Such NGS technologies (also known as massively parallel sequencing technologies) are able to deliver nucleic acid sequence information of a whole genome, exome, transcriptome (all transcribed sequences of a genome) or methylome (all methylated sequences of a genome) in very short time periods, e.g. within 1-2 weeks, for example, within 1-7 days or within less than 24 hours and allow single cell sequencing approaches. Multiple NGS platforms which are commercially available, or which are mentioned in the literature can be used in the context of the present disclosure e.g. those described in detail in WO 2012/159643.

In certain embodiments, the peptide described herein can comprise, but is not limited to, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 31, about 32, about 33, about 34, about 35, about 36, about 37, about 38, about 39, about 40, about 41, about 42, about 43, about 44, about 45, about 46, about 47, about 48, about 49, about 50, about 60, about 70, about 80, about 90, about 100, about 110, about 120, about 150, about 200, about 300, about 350, about 400, about 450, about 500, about 600, about 700, about 800, about 900, about 1,000, about 1,500, about 2,000, about 2,500, about 3,000, about 4,000, about 5,000, about 7,500, about 10,000 amino acids or greater amino acid residues, and any range derivable therein. In specific embodiments, a neoantigenic peptide molecule is equal to or less than 100 amino acids.

In some embodiments, the peptides can be from about 8 and about 50 amino acid residues in length, or from about 8 and about 30, from about 8 and about 20, from about 8 and about 18, from about 8 and about 15, or from about 8 and about 12 amino acid residues in length. In some embodiments, the peptides can be from about 8 and about 500 amino acid residues in length, or from about 8 and about 450, from about 8 and about 400, from about 8 and about 350, from about 8 and about 300, from about 8 and about 250, from about 8 and about 200, from about 8 and about 150, from about 8 and about 100, from about 8 and about 50, or from about 8 and about 30 amino acid residues in length.

In some embodiments, the peptides can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or more amino acid residues in length. In some embodiments, the peptides can be at least 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or more amino acid residues in length. In some embodiments, the peptides can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, or less amino acid residues in length. In some embodiments, the peptides can be at most 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 70, 80, 90, 100, 150, 200, 250, 300, 350, 400, 450, 500, or less amino acid residues in length.

In some embodiments, the peptides has a total length of at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 40, at least 50, at least 60, at least 70, at least 80, at least 90, at least 100, at least 150, at least 200, at least 250, at least 300, at least 350, at least 400, at least 450, or at least 500 amino acids.

In some embodiments, the peptides has a total length of at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 40, at most 50, at most 60, at most 70, at most 80, at most 90, at most 100, at most 150, at most 200, at most 250, at most 300, at most 350, at most 400, at most 450, or at most 500 amino acids.

A longer peptide can be designed in several ways. In some embodiments, when HLA-binding peptides are predicted or known, a longer peptide comprises (1) individual binding peptides with extensions of 2-5 amino acids toward the N- and C-terminus of each corresponding gene product; or (2) a concatenation of some or all of the binding peptides with extended sequences for each. In other embodiments, when sequencing reveals a long (>10 residues) neoepitope sequence present in the tumor (e.g., due to a frameshift, read-through or intron inclusion that leads to a novel peptide sequence), a longer peptide could consist of the entire stretch of novel tumor-specific amino acids as either a single longer peptide or several overlapping longer peptides. In some embodiments, use of a longer peptide is presumed to allow for endogenous processing by patient cells and can lead to more effective antigen presentation and induction of T cell responses. In some embodiments, two or more peptides can be used, where the peptides overlap and are tiled over the long neoantigenic peptide.

In some embodiments, the peptides can have a pI value of from about 0.5 to about 12, from about 2 to about 10, or from about 4 to about 8. In some embodiments, the peptides can have a pI value of at least 4.5, 5, 5.5, 6, 6.5, 7, 7.5, or more. In some embodiments, the peptides can have a pI value of at most 4.5, 5, 5.5, 6, 6.5, 7, 7.5, or less.

In some embodiments, the peptide described herein can be in solution, lyophilized, or can be in crystal form. In some embodiments, the peptide described herein can be prepared synthetically, by recombinant DNA technology or chemical synthesis, or can be isolated from natural sources such as native tumors or pathogenic organisms. Neoepitopes can be synthesized individually or joined directly or indirectly in the peptide. Although the peptide described herein can be substantially free of other naturally occurring host cell proteins and fragments thereof, in some embodiments, the peptide can be synthetically conjugated to be joined to native fragments or particles.

In some embodiments, the peptide described herein can be prepared in a wide variety of ways. In some embodiments, the peptides can be expressed in a host cell, such as a bacteria. In some embodiments, the peptides can be synthesized in solution or on a solid support according to conventional techniques. Various automatic synthesizers are commercially available and can be used according to known protocols. See, for example, Stewart & Young, Solid Phase Peptide Synthesis, 2d. Ed., Pierce Chemical Co., 1984. Further, individual peptides can be joined using chemical ligation to produce larger peptides that are still within the bounds of the present disclosure.

Alternatively, recombinant DNA technology can be employed wherein a nucleotide sequence which encodes the peptide inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1989). Thus, recombinant peptides, which comprise one or more neoantigenic peptides described herein, can be used to present the appropriate T cell epitope.

In some embodiments, the peptide is encoded by a gene with a point mutation resulting in an amino acid substitution of the native peptide. In some embodiments, the peptide is encoded by a gene with a point mutation resulting in frame shift mutation. A frameshift occurs when a mutation disrupts the normal phase of a gene's codon periodicity (also known as “reading frame”), resulting in the translation of a non-native protein sequence. It is possible for different mutations in a gene to achieve the same altered reading frame. In some embodiments, the peptide is encoded by a gene with a mutation resulting in fusion polypeptide, in-frame deletion, insertion, expression of endogenous retroviral polypeptides, and tumor-specific overexpression of polypeptides. In some embodiments, the peptide is encoded by a fusion of a first gene with a second gene. In some embodiments, the peptide is encoded by an in-frame fusion of a first gene with a second gene. In some embodiments, the peptide is encoded by a fusion of a first gene with an exon of a splice variant of the first gene. In some embodiments, the peptide is encoded by a fusion of a first gene with a cryptic exon of the first gene. In some embodiments, the peptide is encoded by a fusion of a first gene with a second gene, wherein the peptide comprises an amino acid sequence encoded by an out of frame sequence resulting from the fusion.

In some aspects, the present disclosure provides a composition comprising at least two or more than two peptides. In some embodiments, the composition described herein contains at least two distinct peptides. In some embodiments, the composition described herein contains a first peptide comprising a first neoepitope and a second peptide comprising a second neoepitope. In some embodiments, the first and second peptides are derived from the same protein. The at least two distinct peptides may vary by length, amino acid sequence or both. The peptides can be derived from any protein known to or have been found to contain a tumor specific mutation. In some embodiments, the composition described herein comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, the composition described herein comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

In some embodiments, the peptide can be derived from a protein with a substitution mutation, e.g., the KRAS G12C, G12D, G12V, Q61H or Q61L mutation, or the NRAS Q61K or Q61R mutation. The substitution may be positioned anywhere along the length of the peptide. For example, it can be located in the N terminal third of the peptide, the central third of the peptide or the C terminal third of the peptide. In another embodiment, the substituted residue is located 2-5 residues away from the N terminal end or 2-5 residues away from the C terminal end. The peptides can be similarly derived from tumor specific insertion mutations where the peptide comprises one or more, or all of the inserted residues. In some embodiments, the first neoepitope and/or the second neoepitope binds to an HLA protein with a greater affinity than a corresponding neoepitope without the substitution. In some embodiments, the first neoepitope and/or the second neoepitope binds to an HLA protein with a greater affinity than a corresponding wild-type sequence without the substitution.

In some embodiments, the first peptide comprises at least one an additional mutation. In some embodiments, one or more of the at least one additional mutation is not a mutation in the first neoepitope. In some embodiments, one or more of the at least one additional mutation is a mutation in the first neoepitope. In some embodiments, the second peptide comprises at least one additional mutation. In some embodiments, one or more of the at least one additional mutation is not a mutation in the second neoepitope. In some embodiments, one or more of the at least one additional mutation is a mutation in the second neoepitope.

In some aspects, the present disclosure provides a composition comprising a single polypeptide comprises the first peptide and the second peptide, or a single polynucleotide encodes the first peptide and the second peptide. In some embodiments, the composition provided herein comprises one or more additional peptides, wherein the one or more additional peptides comprise a third neoepitope. In some embodiments, the first peptide and the second peptide are encoded by a sequence transcribed from the same transcription start site. In some embodiments, the first peptide is encoded by a sequence transcribed from a first transcription start site and the second peptide is encoded by a sequence transcribed from a second transcription start site. In some embodiments, wherein the polypeptide has a length of at least 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the polypeptide comprises a first sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence; and a second sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence. In some embodiments, the polypeptide comprises a first sequence of at least 8 or 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence; and a second sequence of at least 16 or 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence.

In some embodiments, the second peptide is longer than the first peptide. In some embodiments, the first peptide is longer than the second peptide. In some embodiments, the first peptide has a length of at least 9; 10; 11; 12; 13; 14; 15; 16; 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the second peptide has a length of at least 17; 18; 19; 20; 21; 22; 23; 24; 25; 26; 27; 28; 29; 30; 40; 50; 60; 70; 80; 90; 100; 150; 200; 250; 300; 350; 400; 450; 500; 600; 700; 800; 900; 1,000; 1,500; 2,000; 2,500; 3,000; 4,000; 5,000; 7,500; or 10,000 amino acids. In some embodiments, the first peptide comprises a sequence of at least 9 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a corresponding wild-type sequence. In some embodiments, the second peptide comprises a sequence of at least 17 contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a corresponding wild-type sequence.

In some embodiments, the first peptide, the second peptide or both comprise at least one flanking sequence, wherein the at least one flanking sequence is upstream or downstream of the neoepitope. In some embodiments, the at least one flanking sequence has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild-type sequence. In some embodiments, the at least one flanking sequence comprises a non-wild-type sequence. In some embodiments, the at least one flanking sequence is a N-terminus flanking sequence. In some embodiments, the at least one flanking sequence is a C-terminus flanking sequence. In some embodiments, the at least one flanking sequence of the first peptide has at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the at least one flanking sequence of the second peptide. In some embodiments, the at least one flanking region of the first peptide is different from the at least one flanking region of the second peptide. In some embodiments, the at least one flanking residue comprises the mutation.

In some embodiments, a peptide comprises a neoepitope sequence comprising at least one mutant amino acid. In some embodiments, a peptide comprises a neoepitope sequence comprising at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more mutant amino acids. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid. In some embodiments, a peptide comprises a neoepitope sequence derived from a protein comprising at least one mutant amino acid and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid. In some embodiments, a peptide comprises a neoepitope sequence derived from a protein comprising at least one mutant amino acid; at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids upstream of the least one mutant amino acid; and at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more non-mutant amino acids downstream of the least one mutant amino acid.

In some embodiments, a peptide comprises a neoepitope sequence derived from a protein comprising at least one mutant amino acid and a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid, a sequence upstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

In some embodiments, a peptide comprises a neoepitope sequence derived from a protein comprising at least one mutant amino acid and a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence. In some embodiments, a peptide comprises a neoepitope sequence derived from a protein comprising at least one mutant amino acid, a sequence upstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence, and a sequence downstream of the least one mutant amino acid comprising least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more contiguous amino acids with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a corresponding wild type sequence.

### Peptide Modification

In some embodiments, the present disclosure includes modified peptides. A modification can include a covalent chemical modification that does not alter the primary amino acid sequence of the antigenic peptide itself. Modifications can produce peptides with desired properties, for example, prolonging the in vivo half-life, increasing the stability, reducing the clearance, altering the immunogenicity or allergenicity, enabling the raising of particular antibodies, cellular targeting, antigen uptake, antigen processing, HLA affinity, HLA stability or antigen presentation. In some embodiments, a peptide may comprise one or more sequences that enhance processing and presentation of epitopes by APCs, for example, for generation of an immune response.

In some embodiments, the peptide may be modified to provide desired attributes. For instance, the ability of the peptides to induce CTL activity can be enhanced by linkage to a sequence which contains at least one epitope that is capable of inducing a T helper cell response. In some embodiments, immunogenic peptides/T helper conjugates are linked by a spacer molecule. In some embodiments, a spacer comprises relatively small, neutral molecules, such as amino acids or amino acid mimetics, which are substantially uncharged under physiological conditions. Spacers can be selected from, e.g., Ala, Gly, or other neutral spacers of nonpolar amino acids or neutral polar amino acids. It will be understood that the optionally present spacer need not be comprised of the same residues and thus may be a hetero- or homo-oligomer. The neoantigenic peptide may be linked to the T helper peptide either directly or via a spacer either at the amino or carboxy terminus of the peptide. The amino terminus of either the neoantigenic peptide or the T helper peptide may be acylated. Examples of T helper peptides include tetanus toxoid residues 830-843, influenza residues 307-319, and malaria circumsporozoite residues 382-398 and residues 378-389.

The peptide sequences of the present disclosure may optionally be altered through changes at the DNA level, particularly by mutating the DNA encoding the peptide at preselected bases such that codons are generated that will translate into the desired amino acids.

The peptide may also be modified by extending or decreasing the compound's amino acid sequence, e.g., by the addition or deletion of amino acids. The peptides or analogs can also be modified by altering the order or composition of certain residues. It will be appreciated by the skilled artisan that certain amino acid residues essential for biological activity, e.g., those at critical contact sites or conserved residues, may generally not be altered without an adverse effect on biological activity.

In some embodiments, the peptide may be modified using a series of peptides with single amino acid substitutions to determine the effect of electrostatic charge, hydrophobicity, etc. on HLA binding. For instance, a series of positively charged (e.g., Lys or Arg) or negatively charged (e.g., Glu) amino acid substitutions may be made along the length of the peptide revealing different patterns of sensitivity towards various HLA molecules and T cell receptors. In addition, multiple substitutions using small, relatively neutral moieties such as Ala, Gly, Pro, or similar residues may be employed. The substitutions may be homo-oligomers or hetero-oligomers. The number and types of residues which are substituted or added depend on the spacing necessary between essential contact points and certain functional attributes which are sought (e.g., hydrophobicity versus hydrophilicity). Increased binding affinity for an HLA molecule or T cell receptor may also be achieved by such substitutions, compared to the affinity of the parent peptide. In any event, such substitutions should employ amino acid residues or other molecular fragments chosen to avoid, for example, steric and charge interference which might disrupt binding. Amino acid substitutions are typically of single residues. Substitutions, deletions, insertions or any combination thereof may be combined to arrive at a final peptide.

In some embodiments, a peptide described herein can be modified by terminal-NH₂ acylation, e.g., by alkanoyl (C₁-C₂₀) or thioglycolyl acetylation, terminal-carboxyl amidation, e.g., ammonia, methylamine, etc. In some embodiments these modifications can provide sites for linking to a support or other molecule. In some embodiments, the peptide described herein can contain modifications such as but not limited to glycosylation, side chain oxidation, biotinylation, phosphorylation, addition of a surface active material, e.g. a lipid, or can be chemically modified, e.g., acetylation, etc. Moreover, bonds in the peptide can be other than peptide bonds, e.g., covalent bonds, ester or ether bonds, disulfide bonds, hydrogen bonds, ionic bonds, etc.

In some embodiments, a peptide described herein can comprise carriers such as those well known in the art, e.g., thyroglobulin, albumins such as human serum albumin, tetanus toxoid, polyamino acid residues such as poly L-lysine and poly L-glutamic acid, influenza virus proteins, hepatitis B virus core protein, and the like.

The peptides can be further modified to contain additional chemical moieties not normally part of a protein. Those derivatized moieties can improve the solubility, the biological half-life, absorption of the protein, or binding affinity. The moieties can also reduce or eliminate any desirable side effects of the peptides and the like. An overview for those moieties can be found in Remington's Pharmaceutical Sciences, 20th ed., Mack Publishing Co., Easton, PA (2000). For example, neoantigenic peptides having the desired activity may be modified as necessary to provide certain desired attributes, e.g. improved pharmacological characteristics, while increasing or at least retaining substantially all of the biological activity of the unmodified peptide to bind the desired HLA molecule and activate the appropriate T cell. For instance, the peptide may be subject to various changes, such as substitutions, either conservative or non-conservative, where such changes might provide for certain advantages in their use, such as improved HLA binding. Such conservative substitutions may encompass replacing an amino acid residue with another amino acid residue that is biologically and/or chemically similar, e.g., one hydrophobic residue for another, or one polar residue for another. The effect of single amino acid substitutions may also be probed using D-amino acids. Such modifications may be made using well known peptide synthesis procedures, as described in e.g., Merrifield, Science 232:341-347 (1986), Barany & Merrifield, The Peptides, Gross & Meienhofer, eds. (N.Y., Academic Press), pp. 1-284 (1979); and Stewart & Young, Solid Phase Peptide Synthesis, (Rockford, III., Pierce), 2d Ed. (1984).

In some embodiments, the peptide described herein may be conjugated to large, slowly metabolized macromolecules such as proteins; polysaccharides, such as sepharose, agarose, cellulose, cellulose beads; polymeric amino acids such as polyglutamic acid, polylysine; amino acid copolymers; inactivated virus particles; inactivated bacterial toxins such as toxoid from diphtheria, tetanus, cholera, leukotoxin molecules; inactivated bacteria; and dendritic cells.

Changes to the peptide that may include, but are not limited to, conjugation to a carrier protein, conjugation to a ligand, conjugation to an antibody, PEGylation, polysialylation HESylation, recombinant PEG mimetics, Fc fusion, albumin fusion, nanoparticle attachment, nanoparticulate encapsulation, cholesterol fusion, iron fusion, acylation, amidation, glycosylation, side chain oxidation, phosphorylation, biotinylation, the addition of a surface active material, the addition of amino acid mimetics, or the addition of unnatural amino acids.

Additional suitable components and molecules for conjugation include, for example, molecules for targeting to the lymphatic system, thyroglobulin; albumins such as human serum albumin (HAS); tetanus toxoid; Diphtheria toxoid; polyamino acids such as poly(D-lysine:D-glutamic acid); VP6 polypeptides of rotaviruses; influenza virus hemagglutinin, influenza virus nucleoprotein; Keyhole Limpet Hemocyanin (KLH); and hepatitis B virus core protein and surface antigen; or any combination of the foregoing.

Another type of modification is to conjugate (e.g., link) one or more additional components or molecules at the N- and/or C-terminus of a polypeptide sequence, such as another protein (e.g., a protein having an amino acid sequence heterologous to the subject protein), or a carrier molecule. Thus, an exemplary polypeptide sequence can be provided as a conjugate with another component or molecule. In some embodiments, fusion of albumin to the peptide or protein of the present disclosure can, for example, be achieved by genetic manipulation, such that the DNA coding for HSA, or a fragment thereof, is joined to the DNA coding for the one or more polypeptide sequences. Thereafter, a suitable host can be transformed or transfected with the fused nucleotide sequences in the form of, for example, a suitable plasmid, so as to express a fusion polypeptide. The expression may be effected in vitro from, for example, prokaryotic or eukaryotic cells, or in vivo from, for example, a transgenic organism. In some embodiments of the present disclosure, the expression of the fusion protein is performed in mammalian cell lines, for example, CHO cell lines. Furthermore, albumin itself may be modified to extend its circulating half-life. Fusion of the modified albumin to one or more polypeptides can be attained by the genetic manipulation techniques described above or by chemical conjugation; the resulting fusion molecule has a half-life that exceeds that of fusions with non-modified albumin (see, e.g., WO2011/051489). Several albumin-binding strategies have been developed as alternatives for direct fusion, including albumin binding through a conjugated fatty acid chain (acylation). Because serum albumin is a transport protein for fatty acids, these natural ligands with albumin-binding activity have been used for half-life extension of small protein therapeutics.

Additional candidate components and molecules for conjugation include those suitable for isolation or purification. Non-limiting examples include binding molecules, such as biotin (biotin-avidin specific binding pair), an antibody, a receptor, a ligand, a lectin, or molecules that comprise a solid support, including, for example, plastic or polystyrene beads, plates or beads, magnetic beads, test strips, and membranes. Purification methods such as cation exchange chromatography may be used to separate conjugates by charge difference, which effectively separates conjugates into their various molecular weights. The content of the fractions obtained by cation exchange chromatography may be identified by molecular weight using conventional methods, for example, mass spectroscopy, SDS-PAGE, or other known methods for separating molecular entities by molecular weight.

In some embodiments, the amino- or carboxyl-terminus of the peptide or protein sequence of the present disclosure can be fused with an immunoglobulin Fc region (e.g., human Fc) to form a fusion conjugate (or fusion molecule). Fc fusion conjugates have been shown to increase the systemic half-life of biopharmaceuticals, and thus the biopharmaceutical product may require less frequent administration. Fc binds to the neonatal Fc receptor (FcRn) in endothelial cells that line the blood vessels, and, upon binding, the Fc fusion molecule is protected from degradation and re-released into the circulation, keeping the molecule in circulation longer. This Fc binding is believed to be the mechanism by which endogenous IgG retains its long plasma half-life. More recent Fc-fusion technology links a single copy of a biopharmaceutical to the Fc region of an antibody to optimize the pharmacokinetic and pharmacodynamics properties of the biopharmaceutical as compared to traditional Fc-fusion conjugates.

The present disclosure contemplates the use of other modifications, currently known or developed in the future, of the peptides to improve one or more properties. One such method for prolonging the circulation half-life, increasing the stability, reducing the clearance, or altering the immunogenicity or allergenicity of the peptide.

Peptide stability can be assayed in a number of ways. For instance, peptidases and various biological media, such as human plasma and serum, have been used to test stability. See, e.g., Verhoef, et al., Eur. J. Drug Metab. Pharmacokinetics 11:291 (1986). Half-life of the peptides described herein is conveniently determined using a 25% human serum (v/v) assay. The protocol is as follows: pooled human serum (Type AB, non-heat inactivated) is dilapidated by centrifugation before use. The serum is then diluted to 25% with RPMI-1640 or another suitable tissue culture medium. At predetermined time intervals, a small amount of reaction solution is removed and added to either 6% aqueous trichloroacetic acid (TCA) or ethanol. The cloudy reaction sample is cooled (4° C.) for 15 minutes and then spun to pellet the precipitated serum proteins. The presence of the peptides is then determined by reversed-phase HPLC using stability-specific chromatography conditions.

Issues associated with short plasma half-life or susceptibility to protease degradation may be overcome by various modifications, including conjugating or linking the peptide or protein sequence to any of a variety of non-proteinaceous polymers, e.g., polyethylene glycol (PEG), polypropylene glycol, or polyoxyalkylenes (see, for example, typically via a linking moiety covalently bound to both the protein and the nonproteinaceous polymer, e.g., a PEG). Such PEG conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity.

PEGs suitable for conjugation to a polypeptide or protein sequence are generally soluble in water at room temperature, and have the general formula R—(O—CH₂—CH₂)ₙ—O—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. The present disclosure also contemplates compositions of conjugates wherein the PEGs have different n values and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. For example, cation exchange chromatography may be used to separate conjugates, and a fraction is then identified which contains the conjugate having, for example, the desired number of PEGs attached, purified free from unmodified protein sequences and from conjugates having other numbers of PEGs attached.

PEG may be bound to the peptide or protein of the present disclosure via a terminal reactive group (a “spacer”). The spacer is, for example, a terminal reactive group which mediates a bond between the free amino or carboxyl groups of one or more of the polypeptide sequences and PEG. The PEG having the spacer which may be bound to the free amino group includes N-hydroxysuccinylimide PEG which may be prepared by activating succinic acid ester of PEG with N-hydroxysuccinylimide. Another activated PEG which may be bound to a free amino group is 2,4-bis(O-methoxypolyethyleneglycol)-6-chloro-s-triazine which may be prepared by reacting PEG monomethyl ether with cyanuric chloride. The activated PEG which is bound to the free carboxyl group includes polyoxyethylenediamine.

Conjugation of one or more of the peptide or protein sequences of the present disclosure to PEG having a spacer may be carried out by various conventional methods. For example, the conjugation reaction can be carried out in solution at a pH of from 5 to 10, at temperature from 4° C. to room temperature, for 30 minutes to 20 hours, utilizing a molar ratio of reagent to peptide/protein of from 4:1 to 30:1. Reaction conditions may be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art may be used to terminate the reaction. In some embodiments the reaction is terminated by acidifying the reaction mixture and freezing at, e.g., −20° C.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix XTEN technology; Mountain View, CA). This obviates the need for an additional conjugation step during the manufacturing process. Moreover, established molecular biology techniques enable control of the side chain composition of the polypeptide chains, allowing optimization of immunogenicity and manufacturing properties.

### Neoepitopes

A neoepitope comprises a neoantigenic determinant part of a neoantigenic peptide or neoantigenic polypeptide that is recognized by immune system. A neoepitope refers to an epitope that is not present in a reference, such as a non-diseased cell, e.g., a non-cancerous cell or a germline cell, but is found in a diseased cell, e.g., a cancer cell. This includes situations where a corresponding epitope is found in a normal non-diseased cell or a germline cell but, due to one or more mutations in a diseased cell, e.g., a cancer cell, the sequence of the epitope is changed so as to result in the neoepitope. The term “neoepitope” is used interchangeably with “tumor specific neoepitope” in the present specification to designate a series of residues, typically L-amino acids, connected one to the other, typically by peptide bonds between the α-amino and carboxyl groups of adjacent amino acids. The neoepitope can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

In some embodiments, neoepitopes described herein for HLA Class I are 13 residues or less in length and usually consist of between about 8 and about 12 residues, particularly 9 or 10 residues. In some embodiments, neoepitopes described herein for HLA Class II are 25 residues or less in length and usually consist of between about 16 and about 25 residues.

In some embodiments, the composition described herein comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, the composition described herein comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

In some embodiments, the first neoepitope binds to a class I HLA protein to form a class I HLA-peptide complex. In some embodiments, the second neoepitope binds to a class II HLA a protein to form a class II HLA-peptide complex. In some embodiments, the second neoepitope binds to a class I HLA protein to form a class I HLA-peptide complex. In some embodiments, the first neoepitope binds to a class II HLA protein to form a class II HLA-peptide complex. In some embodiments, the first neoepitope activates CD8⁺ T cells. In some embodiments, the first neoepitope activates CD4⁺ T cells. In some embodiments, the second neoepitope activates CD4⁺ T cells. In some embodiments, the second neoepitope activates CD8⁺ T cells. In some embodiments, a TCR of a CD4⁺ T cell binds to a class II HLA-peptide complex. In some embodiments, a TCR of a CD8⁺ T cell binds to a class II HLA-peptide complex. In some embodiments, a TCR of a CD8⁺ T cell binds to a class I HLA-peptide complex. In some embodiments, a TCR of a CD4⁺ T cell binds to a class I HLA-peptide complex.

In some embodiments, the second neoepitope is longer than the first neoepitope. In some embodiments, the first neoepitope has a length of at least 8 amino acids. In some embodiments, the first neoepitope has a length of from 8 to 12 amino acids. In some embodiments, the first neoepitope comprises a sequence of at least 8 contiguous amino acids, wherein at least 1 of the 8 contiguous amino acids are different at corresponding positions of a wild-type sequence. In some embodiments, the first neoepitope comprises a sequence of at least 8 contiguous amino acids, wherein at least 2 of the 8 contiguous amino acids are different at corresponding positions of a wild-type sequence. In some embodiments, the second neoepitope has a length of at least 16 amino acids. In some embodiments, the second neoepitope has a length of from 16 to 25 amino acids. In some embodiments, the second neoepitope comprises a sequence of at least 16 contiguous amino acids, wherein at least 1 of the 16 contiguous amino acids are different at corresponding positions of a wild-type sequence. In some embodiments, the second neoepitope comprises a sequence of at least 16 contiguous amino acids, wherein at least 2 of the 16 contiguous amino acids are different at corresponding positions of a wild-type sequence.

In some embodiments, the neoepitopes bind an HLA protein (e.g., HLA class I or HLA class II). In some embodiments, the neoepitopes bind an HLA protein with greater affinity than the corresponding wild-type peptide. In some embodiments, the neoepitope has an IC₅₀ of less than 5,000 nM, less than 1,000 nM, less than 500 nM, less than 100 nM, less than 50 nM, or less.

In some embodiments, the neoepitope can have an HLA binding affinity of between about 1 pM and about 1 mM, about 100 pM and about 500 μM, about 500 pM and about 10 μM, about 1 nM and about 1 μM, or about 10 nM and about 1 μM. In some embodiments, the neoepitope can have an HLA binding affinity of at least 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900, or 1,000 nM, or more. In some embodiments, the neoepitope can have an HLA binding affinity of at most 2, 3, 4, 5, 6, 7, 8, 9, 10, 15, 20, 25, 30, 35, 40, 45, 50, 55, 60, 65, 70, 75, 80, 85, 90, 95, 100, 150, 200, 250, 300, 350, 400, 450, 500, 550, 600, 700, 800, 900, or 1,000 nM.

In some embodiments, the first and/or second neoepitope binds to an HLA protein with a greater affinity than a corresponding wild-type neoepitope. In some embodiments, the first and/or second neoepitope binds to an HLA protein with a K_{D} or an IC₅₀ less than 1,000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class I protein with a K_{D} or an IC₅₀ less than 1,000 nM, 900 nM, 800 nM, 700 nM, 600 nM, 500 nM, 250 nM, 150 nM, 100 nM, 50 nM, 25 nM or 10 nM. In some embodiments, the first and/or second neoepitope binds to an HLA class II protein with a K_{D} or an IC₅₀ less than 2,000 nM, 1,500 nM, 1,000 nM, 900 nM, 800 nM, 700 nM, 600 nM, 500 nM, 250 nM, 150 nM, 100 nM, 50 nM, 25 nM or 10 nM.

In an aspect, the first and/or second neoepitope binds to a protein encoded by an HLA allele expressed by a subject. In another aspect, the mutation is not present in non-cancer cells of a subject. In yet another aspect, the first and/or second neoepitope is encoded by a gene or an expressed gene of a subject's cancer cells.

### Polynucleotides

Alternatively, a nucleic acid (e.g., a polynucleotide) encoding the peptide of the present disclosure may be used to produce the polypeptide using host cells or in vitro, e.g., by in vitro translation. In some embodiments in vitro translation is used to produce the peptide. Also contemplated are polynucleotides encoding polypeptides, e.g., polybodies comprising neoantigenic peptides, that can be used as a therapeutic. The polynucleotide may be, e.g., DNA, cDNA or RNA, either single- and/or double-stranded

Provided herein are neoantigenic polynucleotides encoding each of the neoantigenic peptides described in the present disclosure. The term “polynucleotide”, “nucleotides” or “nucleic acid” is used interchangeably with “mutant polynucleotide”, “mutant nucleotide”, “mutant nucleic acid”, “neoantigenic polynucleotide”, “neoantigenic nucleotide” or “neoantigenic mutant nucleic acid” in the present disclosure. Various nucleic acid sequences can encode the same peptide due to the redundancy of the genetic code. Each of these nucleic acids falls within the scope of the present disclosure. Nucleic acids encoding peptides can be DNA or RNA, for example, mRNA, or a combination of DNA and RNA. In some embodiments, a nucleic acid encoding a peptide is a self-amplifying mRNA (Brito et al., Adv. Genet. 2015; 89:179-233). Any suitable polynucleotide that encodes a peptide described herein falls within the scope of the present disclosure.

The term “RNA” includes and in some embodiments relates to “mRNA.” The term “mRNA” means “messenger-RNA” and relates to a “transcript” which is generated by using a DNA template and encodes a peptide or polypeptide. Typically, an mRNA comprises a 5′-UTR, a protein coding region, and a 3′-UTR. mRNA only possesses limited half-life in cells and in vitro. In some embodiments, the mRNA is self-amplifying mRNA. In the context of the present disclosure, mRNA may be generated by in vitro transcription from a DNA template. The in vitro transcription methodology is known to the skilled person. For example, there is a variety of in vitro transcription kits commercially available.

The stability and translation efficiency of RNA may be modified as required. For example, RNA may be stabilized and its translation increased by one or more modifications having a stabilizing effects and/or increasing translation efficiency of RNA. Such modifications are described, for example, in PCT/EP2006/009448, incorporated herein by reference. In order to increase expression of the RNA used according to the present disclosure, it may be modified within the coding region, i.e., the sequence encoding the expressed peptide or protein, without altering the sequence of the expressed peptide or protein, so as to increase the GC-content to increase mRNA stability and to perform a codon optimization and, thus, enhance translation in cells.

The term “modification” in the context of the RNA used in the present disclosure includes any modification of an RNA which is not naturally present in said RNA. In some embodiments, the RNA does not have uncapped 5′-triphosphates. Removal of such uncapped 5′-triphosphates can be achieved by treating RNA with a phosphatase. In other embodiments, the RNA may have modified ribonucleotides in order to increase its stability and/or decrease cytotoxicity. In some embodiments, 5-methylcytidine can be substituted partially or completely in the RNA, for example, for cytidine. Alternatively, pseudouridine is substituted partially or completely, for example, for uridine.

In some embodiments, the term “modification” relates to providing an RNA with a 5′-cap or 5′-cap analog. The term “5′-cap” refers to a cap structure found on the 5′-end of an mRNA molecule and generally consists of a guanosine nucleotide connected to the mRNA via an unusual 5′ to 5′ triphosphate linkage. In some embodiments, this guanosine is methylated at the 7-position. The term “conventional 5′-cap” refers to a naturally occurring RNA 5′-cap, to the 7-methylguanosine cap (m G). In the context of the present disclosure, the term “5′-cap” includes a 5′-cap analog that resembles the RNA cap structure and is modified to possess the ability to stabilize RNA and/or enhance translation of RNA if attached thereto, in vivo and/or in a cell.

In certain embodiments, an mRNA encoding a neoantigenic peptide of the present disclosure is administered to a subject in need thereof. In some embodiments, the present disclosure provides RNA, oligoribonucleotide, and polyribonucleotide molecules comprising a modified nucleoside, gene therapy vectors comprising same, gene therapy methods and gene transcription silencing methods comprising same. In some embodiments, the mRNA to be administered comprises at least one modified nucleoside.

The polynucleotides encoding peptides described herein can be synthesized by chemical techniques, for example, the phosphotriester method of Matteucci, et al., J. Am. Chem. Soc. 103:3185 (1981). Polynucleotides encoding peptides comprising or consisting of an analog can be made simply by substituting the appropriate and desired nucleic acid base(s) for those that encode the native epitope.

Polynucleotides described herein can comprise one or more synthetic or naturally-occurring introns in the transcribed region. The inclusion of mRNA stabilization sequences and sequences for replication in mammalian cells can also be considered for increasing polynucleotide expression. In addition, a polynucleotide described herein can comprise immunostimulatory sequences (ISSs or CpGs). These sequences can be included in the vector, outside the polynucleotide coding sequence to enhance immunogenicity.

In some embodiments, the polynucleotides may comprise the coding sequence for the peptide or protein fused in the same reading frame to a polynucleotide which aids, for example, in expression and/or secretion of the peptide or protein from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide.

In some embodiments, the polynucleotides can comprise the coding sequence for the peptide or protein fused in the same reading frame to a marker sequence that allows, for example, for purification of the encoded peptide, which may then be incorporated into a personalized disease vaccine or immunogenic composition. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. Additional tags include, but are not limited to, Calmodulin tags, FLAG tags, Myc tags, S tags, SBP tags, Softag 1, Softag 3, V5 tag, Xpress tag, Isopeptag, SpyTag, Biotin Carboxyl Carrier Protein (BCCP) tags, GST tags, fluorescent protein tags (e.g., green fluorescent protein tags), maltose binding protein tags, Nus tags, Strep-tag, thioredoxin tag, TC tag, Ty tag, and the like. Polynucleotides encoding neoantigenic peptides described herein can also comprise a sequence encoding a ubiquitination signal sequence, and/or a targeting sequence such as an endoplasmic reticulum (ER) signal sequence to facilitate movement of the resulting peptide into the endoplasmic reticulum.

In some embodiments, the polynucleotides may comprise the coding sequence for one or more the presently described peptides or proteins fused in the same reading frame to create a single concatamerized neoantigenic peptide construct capable of producing multiple neoantigenic peptides.

In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. See, e.g. Zoeller et al., Proc. Nat'l. Acad. Sci. USA 81:5662-5066 (1984) and U.S. Pat. No. 4,588,585. In some embodiments, a DNA sequence encoding the peptide or protein of interest would be constructed by chemical synthesis using an oligonucleotide synthesizer. Such oligonucleotides can be designed based on the amino acid sequence of the desired peptide and selecting those codons that are favored in the host cell in which the recombinant polypeptide of interest is produced. Standard methods can be applied to synthesize an isolated polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly

Once assembled (e.g., by synthesis, site-directed mutagenesis, or another method), the polynucleotide sequences encoding a particular isolated polypeptide of interest is inserted into an expression vector and optionally operatively linked to an expression control sequence appropriate for expression of the protein in a desired host. Proper assembly can be confirmed by nucleotide sequencing, restriction mapping, and expression of a biologically active polypeptide in a suitable host. As well known in the art, in order to obtain high expression levels of a transfected gene in a host, the gene can be operatively linked to transcriptional and translational expression control sequences that are functional in the chosen expression host.

Thus, the present disclosure is also directed to vectors, and expression vectors useful for the production and administration of the neoantigenic peptides and neoepitopes described herein, and to host cells comprising such vectors.

### Vectors

In some embodiments, an expression vector capable of expressing the peptide or protein as described herein can also be prepared. Expression vectors for different cell types are well known in the art and can be selected without undue experimentation. Generally, the DNA is inserted into an expression vector, such as a plasmid, in proper orientation and correct reading frame for expression. If necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques (see, e.g., Sambrook et al. (1989) Molecular Cloning, A Laboratory Manual, Cold Spring Harbor Laboratory, Cold Spring Harbor, N.Y.).

A large number of vectors and host systems suitable for producing and administering a neoantigenic peptide described herein are known to those of skill in the art, and are commercially available. The following vectors are provided by way of example. Bacterial: pQE70, pQE60, pQE-9 (Qiagen), pBS, pD10, phagescript, psiX174, pBluescript SK, pbsks, pNH8A, pNH16a, pNH18A, pNH46A (Stratagene); ptrc99a, pKK223-3, pKK233-3, pDR540, pRIT5 (Pharmacia); pCR (Invitrogen). Eukaryotic: pWLNEO, pSV2CAT, pOG44, pXT1, pSG (Stratagene) pSVK3, pBPV, pMSG, pSVL (Pharmacia); p75.6 (Valentis); pCEP (Invitrogen); pCEI (Epimmune). However, any other plasmid or vector can be used as long as it is replicable and viable in the host.

For expression of the neoantigenic peptides described herein, the coding sequence will be provided operably linked start and stop codons, promoter and terminator regions, and in some embodiments, and a replication system to provide an expression vector for expression in the desired cellular host. For example, promoter sequences compatible with bacterial hosts are provided in plasmids containing convenient restriction sites for insertion of the desired coding sequence. The resulting expression vectors are transformed into suitable bacterial hosts.

Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. Such promoters can also be derived from viral sources, such as, e.g., human cytomegalovirus (CMV-IE promoter) or herpes simplex virus type-1 (HSV TK promoter). Nucleic acid sequences derived from the SV40 splice, and polyadenylation sites can be used to provide the required nontranscribed genetic elements.

Recombinant expression vectors may be used to amplify and express DNA encoding the peptide or protein as described herein. Recombinant expression vectors are replicable DNA constructs which have synthetic or cDNA-derived DNA fragments encoding a peptide or a bioequivalent analog operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences, as described in detail herein. Such regulatory elements can include an operator sequence to control transcription. The ability to replicate in a host, usually conferred by an origin of replication, and a selection gene to facilitate recognition of transformants can additionally be incorporated. DNA regions are operatively linked when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. Generally, operatively linked means contiguous, and in the case of secretory leaders, means contiguous and in reading frame. Structural elements intended for use in yeast expression systems include a leader sequence enabling extracellular secretion of translated protein by a host cell. Alternatively, where recombinant protein is expressed without a leader or transport sequence, it can include an N-terminal methionine residue. This residue can optionally be subsequently cleaved from the expressed recombinant protein to provide a final product.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), acid phosphatase, or heat shock proteins, among others. The heterologous structural sequence is assembled in appropriate phase with translation initiation and termination sequences, and in some embodiments, a leader sequence capable of directing secretion of translated protein into the periplasmic space or extracellular medium. Optionally, the heterologous sequence can encode a fusion protein including an N-terminal identification peptide imparting desired characteristics, e.g., stabilization or simplified purification of expressed recombinant product.

In some embodiments, the neoantigenic peptide described herein can also be expressed by bacterial vectors. Examples of bacterial expression vectors include the most commonly used E. coli expression system. Escherichia coli (E. coli) is one of the most widely used hosts for the production of heterologous proteins and its genetics are far better characterized than those of any other microorganism. Fused proteins can be expressed in E. coli expression vectors and grown in various hosting strains. E. coli strains DH5α, JM101, RRI, DH5c, S17-1 λ/pir, K12, Top10, and BL21(DE3), are exemplary strains that can be used for the heterologous gene expression host. Suitable bacteria will in many embodiments comprise one or more mutations or other genetic modifications that increase the expression levels.

Molecular cloning techniques to achieve these ends are known in the art. A wide variety of cloning and in vitro amplification methods suitable for the construction of recombinant nucleic acids such as expression vectors are well-known to persons of skill. Examples of these techniques and instructions sufficient to direct persons of skill through many cloning exercises are found in Sambrook J. et al., Molecular Cloning: A Laboratory Manual, ed. 4, Cold Spring Harbor Laboratory Press; Berger and Kimmel, Guide to Molecular Cloning Techniques, Methods in Enzymology volume 152 Academic Press, Inc., San Diego, CA (Berger); and Current Protocols in Molecular Biology, F. M. Ausubel et al., eds., Current Protocols, a joint venture between Greene Publishing Associates, Inc. and John Wiley Sons, Inc., (1994 Supplement) (Ausubel). Examples of protocols sufficient to direct persons of skill through in vitro amplification methods, including the polymerase chain reaction (PCR) the ligase chain reaction (LCR), Qp-replicase amplification and other RNA polymerase mediated techniques are found in Berger, Sambrook, and Ausubel, as well as Mullis et al. (1987) U.S. Pat. No. 4,683,202;

In brief, the method of protein expression in an E. coli system includes the following steps: A nucleic acid encoding the protein or polypeptide of interest is cloned into the desired plasmid. The plasmid is then incorporated into the bacterial host, (e.g. by transfection, or electroporation or any other suitable method known to one of skill in the art) that is compatible for growth of the plasmid. The bacteria is cultured and expanded in a bacterial culture medium, while it amplifies the protein product encoded by the nucleic acid within the bacteria (or is secreted in the culture medium if the encoded protein is a secreted protein. The protein is then harvested from the expanded bacteria (for endogenous proteins) or the culture supernatant (for secreted proteins). High copy number expression plasmids are primarily selected for the purpose. The basic elements of any plasmid for bacterial cloning comprise inter alia an ORI (origin of replication) site; control elements such as promoter, enhancer, at the 5′ end; one or more expression stabilizer at the 3′end, and selection marker genes, such as antibiotic resistance. Most E. coli strains can be used to propagate plasmids. Expression plasmids often comprise tags for purification of the proteins. When a nucleic acid encoding a protein of interest is cloned within the designated cloning sites of a commercially available vector that has a suitable tag, the expressed protein is a fused protein comprising the tag. For example, plasmid pDEST-HisMBP comprise MBP and hexa-histidine tag. In this case the MBP protein and the His tags are used for isolation and purification of the protein from the E. coli cells. Various such plasmids and expression systems are commercially available and is known to one of skill in the art. Many promoters such as P_{lac}, Pᵤₚ, P_{tac}, λP_{L}, P_{T7} and P_{BAD} are commonly utilized for the construction of expression vectors. Among these, lacUV5, tac and combined system of P_{T7} with lacUV5 are widely used, because the expression can easily be regulated by varying the concentration of the inducer isopropyl-beta-D-thiogalactopyrano-side (IPTG). Other promoters called λPL and λPR are generally induced by a temperature shift. To obtain high level expression of a cloned gene, the expression cassettes can include other sequences such as ribosome binding sites for translational initiation and transcription/translation terminator sequences. High level expression of a desired peptide or polypeptide can be achieved by using bacterial expression vectors containing dual promoters. The cells can be grown in shake flasks or other containers, although for large-scale preparation of the polypeptide growth in a fermenter is preferred. To obtain the maximum level of expression, galactose is added to the nutrient medium at an appropriate time in the growth cycle to induce increased expression of the desired polypeptide. For example, growth of the host cells can be initiated in culture medium containing fructose (0.25% final concentration) as the carbon source; other sugars glycerol, acetate) that cause an increase in intracellular cAMP (adenosine 3′,5′-cyclic monophosphate) concentration can also be used as a carbon source.

To allow selection of cells comprising the constructs, one or more selectable marker genes such as antibiotic-resistance genes are conveniently included in the expression vectors. These genes encode a protein necessary for the survival or growth of transformed host cells grown in a selective culture medium. Host cells not transformed with the vector containing the selection gene will not survive in the culture medium. Typical selection genes encode proteins that confer resistance to antibiotics or other toxins, such as ampicillin, neomycin, kanamycin, chloramphenicol, or tetracycline

Alternatively, selectable markers may encode proteins that complement auxotrophic deficiencies or supply critical nutrients not available from complex media, the gene encoding D-alanine racemase for Bacilli. A number of selectable markers are known to those of skill in the art and are described for instance in Sambrook et al., supra. A preferred selectable marker for use in using the dual tac-lac promoter to express a desired polypeptide is a kanamycin resistance marker (Vieira and Messing, Gene 19: 259 (1982)). Use of kanamycin selection is advantageous over, for example, ampicillin selection because ampicillin is quickly degraded by p-lactamase in culture medium, thus removing selective pressure and allowing the culture to become overgrown with cells that do not contain the vector.

Construction of suitable vectors containing one or more of the above listed components can employ standard ligation techniques as described in the reference cited above. The vectors may comprise other sequences to allow the vector to be cloned in prokaryotic hosts. One of skill will recognize that each of these vector components can be modified without substantially affecting their function.

Isolated plasmids or DNA fragments are cleaved, tailored, and re-ligated in the form desired to generate the plasmids required. To confirm correct sequences in plasmids constructed, the plasmids are analyzed by standard techniques such as by restriction endonuclease digestion, and/or sequencing according to known methods.

Affinity tags and other types of genetically engineered fusion partners are widely employed as tools in molecular biology. Affinity tags are extremely useful to purify expressed proteins. The fundamental steps of purification include protein lysis, binding of the protein to a matrix, washing and elution. As an example, polyhistidine (His6) tags are commonly placed at the N- or C-terminus of the proteins while cloning, the respective protein of interest expressed with the tag, and finally the His can be captured by a metal, as the nitrogen in the imidazole moiety of the polyhistidine interacts with a metal. Typically, the metal is immobilized to support for the capture Washing and elution is performed by suitable buffer. The tag can be so designed to be cleaved out of the protein expressed after purification. Likewise, various other tags like GST-tag, Halo tag, HA-tag etc. are well known by one of skill in the art for the purpose. Tag moieties are typically small and do not interfere with the expressed protein structure. Although these were originally developed to facilitate the detection and purification of recombinant proteins, in recent years it has become clear that certain tags can also improve the yield, enhance the solubility and even promote the proper folding of their fusion partners Insolubility of recombinant proteins can be a major problem particularly in Escherichia coli in the production of bioactive material for structural and functional studies, the utilization of solubility-enhancing tags to avoid the formation of insoluble protein aggregates has been rapidly gaining in popularity. Although many proteins that are highly soluble when overproduced in E. coli have been reported to possess solubility-enhancing qualities as fusion partners, in most cases the evidence to support these claims is scant. Among the known solubility-enhancing fusion partners, MBP is unique in that it is also a natural affinity tag. MBP fusion proteins can be purified using amylose affinity chromatography. Further examples include His tag, FLAG tag, HA tag and others

Examples of expression vectors that can infect a mammalian cell include attenuated viral hosts, such as vaccinia or fowlpox. As an example of this approach, vaccinia virus is used as a vector to express nucleotide sequences that encode the neoantigenic peptides described herein. Vaccinia vectors and methods useful in immunization protocols are described in, e.g., U.S. Pat. No. 4,722,848. Another vector is BCG (Bacille Calmette Guerin). BCG vectors are described by Stover et al., Nature 351:456-460 (1991).

A wide variety of other vectors useful for therapeutic administration or immunization of the neoantigenic polypeptides described herein, e.g. adeno and adeno-associated virus vectors, retroviral vectors, Salmonella typhimurium vectors, detoxified anthrax toxin vectors, Sendai virus vectors, poxvirus vectors, canarypox vectors, and fowlpox vectors, and the like, will be apparent to those skilled in the art from the description herein. In some embodiments, the vector is Modified Vaccinia Ankara (VA) (e.g. Bavarian Noridic (MVA-BN)).

Among vectors that may be used in the practice of the present disclosure, integration in the host genome of a cell is possible with retrovirus gene transfer methods, often resulting in long term expression of the inserted transgene. In some embodiments, the retrovirus is a lentivirus. Additionally, high transduction efficiencies have been observed in many different cell types and target tissues. The tropism of a retrovirus can be altered by incorporating foreign envelope proteins, expanding the potential target population of target cells. A retrovirus can also be engineered to allow for conditional expression of the inserted transgene, such that only certain cell types are infected by the lentivirus. Cell type specific promoters can be used to target expression in specific cell types. Lentiviral vectors are retroviral vectors (and hence both lentiviral and retroviral vectors may be used in the practice of the present disclosure). Moreover, lentiviral vectors are able to transduce or infect non-dividing cells and typically produce high viral titers. Selection of a retroviral gene transfer system may therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the desired nucleic acid into the target cell to provide permanent expression. Widely used retroviral vectors that may be used in the practice of the present disclosure include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., (1992) J. Virol. 66:2731-2739; Johann et al., (1992) J. Virol. 66:1635-1640; Sommnerfelt et al., (1990) Virol. 176:58-59; Wilson et al., (1998) J. Virol. 63:2374-2378; Miller et al., (1991) J. Virol. 65:2220-2224; PCT/US94/05700).

Also useful in the practice of the present disclosure is a minimal non-primate lentiviral vector, such as a lentiviral vector based on the equine infectious anemia virus (EIAV). The vectors may have cytomegalovirus (CMV) promoter driving expression of the target gene. Accordingly, the present disclosure contemplates amongst vector(s) useful in the practice of the present disclosure: viral vectors, including retroviral vectors and lentiviral vectors.

Also useful in the practice of the present disclosure is an adenovirus vector. One advantage is the ability of recombinant adenoviruses to efficiently transfer and express recombinant genes in a variety of mammalian cells and tissues in vitro and in vivo, resulting in the high expression of the transferred nucleic acids. Further, the ability to productively infect quiescent cells, expands the utility of recombinant adenoviral vectors. In addition, high expression levels ensure that the products of the nucleic acids will be expressed to sufficient levels to generate an immune response (see e.g., U.S. Pat. No. 7,029,848, hereby incorporated by reference).

As to adenovirus vectors useful in the practice of the present disclosure, mention is made of U.S. Pat. No. 6,955,808. The adenovirus vector used can be selected from the group consisting of the Ad5, Ad35, Ad11, C6, and C7 vectors. The sequence of the Adenovirus 5 (“Ad5”) genome has been published. (Chroboczek, J., Bieber, F., and Jacrot, B. (1992) The Sequence of the Genome of Adenovirus Type 5 and Its Comparison with the Genome of Adenovirus Type 2, Virology 186, 280-285; the contents if which is hereby incorporated by reference). Ad35 vectors are described in U.S. Pat. Nos. 6,974,695, 6,913,922, and 6,869,794. Ad11 vectors are described in U.S. Pat. No. 6,913,922. C6 adenovirus vectors are described in U.S. Pat. Nos. 6,780,407; 6,537,594; 6,309,647; 6,265,189; 6,156,567; 6,090,393; 5,942,235 and 5,833,975. C7 vectors are described in U.S. Pat. No. 6,277,558. Adenovirus vectors that are E1-defective or deleted, E3-defective or deleted, and/or E4-defective or deleted may also be used. Certain adenoviruses having mutations in the E1 region have improved safety margin because E1-defective adenovirus mutants are replication-defective in non-permissive cells, or, at the very least, are highly attenuated. Adenoviruses having mutations in the E3 region may have enhanced the immunogenicity by disrupting the mechanism whereby adenovirus down-regulates MHC class I molecules. Adenoviruses having E4 mutations may have reduced immunogenicity of the adenovirus vector because of suppression of late gene expression. Such vectors may be particularly useful when repeated re-vaccination utilizing the same vector is desired. Adenovirus vectors that are deleted or mutated in E1, E3, E4; E1 and E3; and E1 and E4 can be used in accordance with the present disclosure.

Furthermore, “gutless” adenovirus vectors, in which all viral genes are deleted, can also be used in accordance with the present disclosure. Such vectors require a helper virus for their replication and require a special human 293 cell line expressing both Ela and Cre, a condition that does not exist in natural environment. Such “gutless” vectors are non-immunogenic and thus the vectors may be inoculated multiple times for re-vaccination. The “gutless” adenovirus vectors can be used for insertion of heterologous inserts/genes such as the transgenes of the present disclosure, and can even be used for co-delivery of a large number of heterologous inserts/genes.

In some embodiments, the delivery is via an adenovirus, which may be at a single booster dose. In some embodiments, the adenovirus is delivered via multiple doses. In terms of in vivo delivery, AAV is advantageous over other viral vectors due to low toxicity and low probability of causing insertional mutagenesis because it doesn't integrate into the host genome. AAV has a packaging limit of 4.5 or 4.75 Kb. Constructs larger than 4.5 or 4.75 Kb result in significantly reduced virus production. There are many promoters that can be used to drive nucleic acid molecule expression. AAV ITR can serve as a promoter and is advantageous for eliminating the need for an additional promoter element.

For ubiquitous expression, the following promoters can be used: CMV, CAG, CBh, PGK, SV40, Ferritin heavy or light chains, etc. For brain expression, the following promoters can be used: Synapsin I for all neurons, CaMK II alpha for excitatory neurons, GAD67 or GAD65 or VGAT for GABAergic neurons, etc. Promoters used to drive RNA synthesis can include: Pol III promoters such as U6 or H1. The use of a Pol II promoter and intronic cassettes can be used to express guide RNA (gRNA). With regard to AAV vectors useful in the practice of the present disclosure, mention is made of U.S. Pat. Nos. 5,658,785, 7,115,391, 7,172,893, 6,953,690, 6,936,466, 6,924,128, 6,893,865, 6,793,926, 6,537,540, 6,475,769 and 6,258,595, and documents cited therein. As to AAV, the AAV can be AAV1, AAV2, AAV5 or any combination thereof. One can select the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. In some embodiments the delivery is via an AAV. The dosage may be adjusted to balance the therapeutic benefit against any side effects.

In some embodiments, a Poxvirus is used in the presently described composition. These include orthopoxvirus, avipox, vaccinia, MVA, NYVAC, canarypox, ALVAC, fowlpox, TROVAC, etc. (see e.g., Verardi et al., Hum. Vaccin. Immunother. 2012 July; 8(7):961-70; and Moss, Vaccine. 2013; 31(39): 4220-4222). Poxvirus expression vectors were described in 1982 and quickly became widely used for vaccine development as well as research in numerous fields. Advantages of the vectors include simple construction, ability to accommodate large amounts of foreign DNA and high expression levels. Information concerning poxviruses that may be used in the practice of the present disclosure, such as Chordopoxvirinae subfamily poxviruses (poxviruses of vertebrates), for instance, orthopoxviruses and avipoxviruses, e.g., vaccinia virus (e.g., Wyeth Strain, WR Strain (e.g., ATCC® VR-1354), Copenhagen Strain, NYVAC, NYVAC.1, NYVAC.2, MVA, MVA-BN), canarypox virus (e.g., Wheatley C93 Strain, ALVAC), fowlpox virus (e.g., FP9 Strain, Webster Strain, TROVAC), dovepox, pigeonpox, quailpox, and raccoon pox, inter alia, synthetic or non-naturally occurring recombinants thereof, uses thereof, and methods for making and using such recombinants may be found in scientific and patent literature.

In some embodiments, the vaccinia virus is used in the disease vaccine or immunogenic composition to express a antigen. (Rolph et al., Recombinant viruses as vaccines and immunological tools. Curr. Opin. Immunol. 9:517-524, 1997). The recombinant vaccinia virus is able to replicate within the cytoplasm of the infected host cell and the polypeptide of interest can therefore induce an immune response. Moreover, Poxviruses have been widely used as vaccine or immunogenic composition vectors because of their ability to target encoded antigens for processing by the major histocompatibility complex class I pathway by directly infecting immune cells, in particular antigen-presenting cells, but also due to their ability to self-adjuvant.

In some embodiments, ALVAC is used as a vector in a disease vaccine or immunogenic composition. ALVAC is a canarypox virus that can be modified to express foreign transgenes and has been used as a method for vaccination against both prokaryotic and eukaryotic antigens (Horig H, Lee D S, Conkright W, et al. Phase I clinical trial of a recombinant canarypoxvirus (ALVAC) vaccine expressing human carcinoembryonic antigen and the B7.1 co-stimulatory molecule. Cancer Immunol. Immunother. 2000; 49:504-14; von Mehren M, Arlen P, Tsang K Y, et al. Pilot study of a dual gene recombinant avipox vaccine containing both carcinoembryonic antigen (CEA) and B7.1 transgenes in patients with recurrent CEA-expressing adenocarcinomas. Clin. Cancer. Res. 2000; 6:2219-28; Musey L, Ding Y, Elizaga M, et al. HIV-1 vaccination administered intramuscularly can induce both systemic and mucosal T cell immunity in HIV-1-uninfected individuals. J. Immunol. 2003; 171: 1094-101; Paoletti E. Applications of pox virus vectors to vaccination: an update. Proc. Natl. Acad. Sci. USA 1996; 93:11349-53; U.S. Pat. No. 7,255,862). In a phase I clinical trial, an ALVAC virus expressing the tumor antigen CEA showed an excellent safety profile and resulted in increased CEA-specific T cell responses in selected patients; objective clinical responses, however, were not observed (Marshall J L, Hawkins M J, Tsang K Y, et al. Phase I study in cancer patients of a replication-defective avipox recombinant vaccine that expresses human carcinoembryonic antigen. J. Clin. Oncol. 1999; 17:332-7).

In some embodiments, a Modified Vaccinia Ankara (MVA) virus may be used as a viral vector for an antigen vaccine or immunogenic composition. MVA is a member of the Orthopoxvirus family and has been generated by about 570 serial passages on chicken embryo fibroblasts of the Ankara strain of Vaccinia virus (CVA) (see, e.g., Mayr, A., et al., Infection 3, 6-14, 1975). As a consequence of these passages, the resulting MVA virus contains 31 kilobases less genomic information compared to CVA, and is highly host cell restricted (Meyer, H. et al., J. Gen. Virol. 72, 1031-1038, 1991). MVA is characterized by its extreme attenuation, namely, by a diminished virulence or infectious ability, but still holds an excellent immunogenicity. When tested in a variety of animal models, MVA was proven to be avirulent, even in immuno-suppressed individuals. Moreover, MVA-BN®-HER2 is a candidate immunotherapy designed for the treatment of HER-2-positive breast cancer and is currently in clinical trials. (Mandl et al., Cancer Immunol. Immunother. January 2012; 61(1): 19-29). Methods to make and use recombinant MVA has been described (e.g., see U.S. Pat. Nos. 8,309,098 and 5,185,146 hereby incorporated in its entirety).

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems could also be employed. Appropriate cloning and expression vectors for use with bacterial, fungal, yeast, and mammalian cellular hosts are well known in the art (see Pouwels et al., Cloning Vectors: A Laboratory Manual, Elsevier, N.Y., 1985).

Various mammalian or insect cell culture systems are also advantageously employed to express recombinant protein. Expression of recombinant proteins in mammalian cells can be performed because such proteins are generally correctly folded, appropriately modified and completely functional. Examples of suitable mammalian host cell lines include the COS-7 lines of monkey kidney cells, described by Gluzman (Cell 23:175, 1981), and other cell lines capable of expressing an appropriate vector including, for example, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences. Baculovirus systems for production of heterologous proteins in insect cells are reviewed by Luckow and Summers, Bio/Technology 6:47 (1988).

Host cells are genetically engineered (transduced or transformed or transfected) with the vectors which can be, for example, a cloning vector or an expression vector. The vector can be, for example, in the form of a plasmid, a viral particle, a phage, etc. The engineered host cells can be cultured in conventional nutrient media modified as appropriate for activating promoters, selecting transformants or amplifying the polynucleotides. The culture conditions, such as temperature, pH and the like, are those previously used with the host cell selected for expression, and will be apparent to the ordinarily skilled artisan.

As representative examples of appropriate hosts, there can be mentioned: bacterial cells, such as E. coli, Bacillus subtilis, Salmonella typhimurium and various species within the genera Pseudomonas, Streptomyces, and Staphylococcus; fungal cells, such as yeast; insect cells such as Drosophila and Sf9; animal cells such as COS-7 lines of monkey kidney fibroblasts, described by Gluzman, Cell 23:175 (1981), and other cell lines capable of expressing a compatible vector, for example, the C127, 3T3, CHO, HeLa and BHK cell lines or Bowes melanoma; plant cells, etc. The selection of an appropriate host is deemed to be within the scope of those skilled in the art from the teachings herein.

Yeast, insect or mammalian cell hosts can also be used, employing suitable vectors and control sequences. Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines.

Polynucleotides described herein can be administered and expressed in human cells (e.g., immune cells, including dendritic cells). A human codon usage table can be used to guide the codon choice for each amino acid. Such polynucleotides comprise spacer amino acid residues between epitopes and/or analogs, such as those described above, or can comprise naturally-occurring flanking sequences adjacent to the epitopes and/or analogs (and/or CTL (e.g., CD8⁺), Th (e.g., CD4⁺), and B cell epitopes).

Standard regulatory sequences well known to those of skill in the art can be included in the vector to ensure expression in the human target cells. Several vector elements are desirable: a promoter with a downstream cloning site for polynucleotide, e.g., minigene insertion; a polyadenylation signal for efficient transcription termination; an E. coli origin of replication; and an E. coli selectable marker (e.g. ampicillin or kanamycin resistance). Numerous promoters can be used for this purpose, e.g., the human cytomegalovirus (hCMV) promoter. See, e.g., U.S. Pat. Nos. 5,580,859 and 5,589,466 for other suitable promoter sequences. In some embodiments, the promoter is the CMV-IE promoter.

Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from Escherichia coli, including pCR1, pBR322, pMB9 and their derivatives, wider host range plasmids, such as M13 and filamentous single-stranded DNA phages.

Vectors may be introduced into animal tissues by a number of different methods. The two most popular approaches are injection of DNA in saline, using a standard hypodermic needle, and gene gun delivery. A schematic outline of the construction of a DNA vaccine plasmid and its subsequent delivery by these two methods into a host is illustrated at Scientific American (Weiner et al., (1999) Scientific American 281 (1): 34-41). Injection in saline is normally conducted intramuscularly (IM) in skeletal muscle, or intradermally (ID), with DNA being delivered to the extracellular spaces. This can be assisted by electroporation by temporarily damaging muscle fibers with myotoxins such as bupivacaine; or by using hypertonic solutions of saline or sucrose (Alarcon et al., (1999). Adv. Parasitol. Advances in Parasitology 42: 343-410). Immune responses to this method of delivery can be affected by many factors, including needle type, needle alignment, speed of injection, volume of injection, muscle type, and age, sex and physiological condition of the animal being injected (Alarcon et al., (1999). Adv. Parasitol. Advances in Parasitology 42: 343-410).

Gene gun delivery, the other commonly used method of delivery, ballistically accelerates plasmid DNA (pDNA) that has been adsorbed onto gold or tungsten microparticles into the target cells, using compressed helium as an accelerant (Alarcon et al., (1999). Adv. Parasitol. Advances in Parasitology 42: 343-410; Lewis et al., (1999). Advances in Virus Research (Academic Press) 54: 129-88).

Alternative delivery methods may include aerosol instillation of naked DNA on mucosal surfaces, such as the nasal and lung mucosa, (Lewis et al., (1999). Advances in Virus Research (Academic Press) 54: 129-88) and topical administration of pDNA to the eye and vaginal mucosa (Lewis et al., (1999) Advances in Virus Research (Academic Press) 54: 129-88). Mucosal surface delivery has also been achieved using cationic liposome-DNA preparations, biodegradable microspheres, attenuated Shigella or Listeria vectors for oral administration to the intestinal mucosa, and recombinant adenovirus vectors. DNA or RNA may also be delivered to cells following mild mechanical disruption of the cell membrane, temporarily permeabilizing the cells. Such a mild mechanical disruption of the membrane can be accomplished by gently forcing cells through a small aperture (Sharei et al., Ex Vivo Cytosolic Delivery of Functional Macromolecules to Immune Cells, PLOS ONE (2015)).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). In the case where a non-viral delivery system is utilized, an exemplary delivery vehicle is a liposome. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., Glycobiology 5: 505-10 (1991)). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.). Stock solutions of lipids in chloroform or chloroform/methanol can be stored at about-20° C. Chloroform is used as the only solvent since it is more readily evaporated than methanol. In some embodiments, lipids are essential for the delivery of nucleic acids encoding proteins of interest into a cell. Nucleic acid constructs encoding fusion polypeptides as described herein can be delivered via lipid compositions, e.g., liposomes inside a cell for expression of the fusion polypeptide. In some embodiments, the liposomes comprise one or more cationic lipids. In some embodiments the liposomes comprise at least a cationic lipid, and at least a non-cationic lipid.

In some embodiments, a vector comprises a polynucleotide encoding a first peptide comprising a first neoepitope and a second peptide comprising a second neoepitope. In some embodiments, the first and second peptides are derived from the same protein. The at least two distinct peptides may vary by length, amino acid sequence or both. The peptides are derived from any protein known to or have been found to contain a tumor specific mutation. In some embodiments, a vector comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, a vector comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

In some embodiments, a vector comprises a polynucleotide operably linked to a promoter. In some embodiments, the vector is a self-amplifying RNA replicon, plasmid, phage, transposon, cosmid, virus, or virion. In some embodiments, the vector is derived from a retrovirus, lentivirus, adenovirus, adeno-associated virus, herpes virus, pox virus, alpha virus, vaccinia virus, hepatitis B virus, human papillomavirus or a pseudotype thereof. In some embodiments, the vector is a non-viral vector. In some embodiments, the non-viral vector is a nanoparticle, a cationic lipid, a cationic polymer, a metallic nanopolymer, a nanorod, a liposome, a micelle, a microbubble, a cell-penetrating peptide, or a liposphere.

### T Cells and T Cell Receptors

A T cell is a T-lymphocyte, an immune system cell that matures in the thymus and produces T cell receptors (TCRs). T cells can be naive, that is, they are not exposed to antigen; exhibit increased expression of CD62L, CCR7, CD28, CD3, CD127, and CD45RA, and decreased expression of CD45RO as compared to more mature T cells with antigenic exposure and memory; memory T cells (T_{M}) (antigen-experienced and long-lived), and effector cells (antigen-experienced, cytotoxic). T_{M} can be further divided into subsets of central memory T cells (TC_{M}, increased expression of CD62L, CCR7, CD28, CD127, CD45RO, and CD95, and decreased expression of CD54RA as compared to naive T cells) and effector memory T cells (T_{EM}, decreased expression of CD62L, CCR7, CD28, CD45RA, and increased expression of CD127 as compared to naive T cells or TC_{M})-Effector T cells (T_{E}) refers to antigen-experienced CD8+ cytotoxic T lymphocytes that have decreased expression of CD62L, CCR7, CD28, and are positive for granzyme and perforin as compared to TC_{M}—Other exemplary T cells include regulatory T cells, such as CD4+CD25+ (Foxp3+) regulatory T cells and Treg17 cells, as well as Tr1, Th3, CD8+CD28−, and Qa-1 restricted T cells.

In one aspect, the present disclosure provides mechanisms for targeting neoantigens to expose naïve T cells. Lymph nodes harbor naïve T cells. Targeting the antigen peptides to the lymph node facilitates exposure of resident naïve T cells to the antigen, and therefore new primed T cells are generated. When the polybodies are prepared with neoantigen-enriched peptides, the T cells that are thereafter primed in the lymph node are primed against neoantigens and are immunogenic to the tumor or cancer.

In another embodiment, the present disclosure provides a composition comprising a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first neoepitope binds to a class I HLA protein to form a class I HLA-peptide complex. In some embodiments, the first neoepitope binds to a class II HLA protein to form a class II HLA-peptide complex. In some embodiments, the second neoepitope binds to a class II HLA a protein to form a class II HLA-peptide complex. In some embodiments, the second neoepitope binds to a class I HLA protein to form a class I HLA-peptide complex. In some embodiments, the first neoepitope activates CD8⁺ T cells. In some embodiments, the first neoepitope activates CD4⁺ T cells. In some embodiments, the second neoepitope activates CD4⁺ T cells. In some embodiments, the second neoepitope activates CD8⁺ T cells. In some embodiments, a TCR of a CD4⁺ T cell binds to a class II HLA-peptide complex. In some embodiments, a TCR of a CD8⁺ T cell binds to a class II HLA-peptide complex. In some embodiments, a TCR of a CD8⁺ T cell binds to a class I HLA-peptide complex. In some embodiments, a TCR of a CD4⁺ T cell binds to a class I HLA-peptide complex.

In some embodiments, the first TCR is a first chimeric antigen receptor specific for the first neoepitope and the second TCR is a second chimeric antigen receptor specific for the second neoepitope. In some embodiments, the first T cell is a cytotoxic T cell. In some embodiments, the first T cell is a gamma delta T cell. In some embodiments, the second T cell is a helper T cell. In some embodiments, the first and/or second TCR binds to an HLA-peptide complex with a K_{D} or an IC₅₀ of less than 1,000 nM, 900 nM, 800 nM, 700 nM, 600 nM, 500 nM, 250 nM, 150 nM, 100 nM, 50 nM, 25 nM or 10 nM. In some embodiments, the first and/or second TCR binds to an HLA class I-peptide complex with a K_{D} or an IC₅₀ of less than 1,000 nM, 900 nM, 800 nM, 700 nM, 600 nM, 500 nM, 250 nM, 150 nM, 100 nM, 50 nM, 25 nM or 10 nM. In some embodiments, the first and/or second TCR binds to an HLA class II-peptide complex with a K_{D} or an IC₅₀ of less than 2,000, 1,500, 1,000 nM, 900 nM, 800 nM, 700 nM, 600 nM, 500 nM, 250 nM, 150 nM, 100 nM, 50 nM, 25 nM or 10 nM.

### Antigen Presenting Cells

The neoantigenic peptide or protein can be provided as antigen presenting cells (e.g., dendritic cells) containing such peptides, proteins or polynucleotides as described herein. In other embodiments, such antigen presenting cells are used to stimulate T cells for use in patients. Thus, one embodiment of the present disclosure is a composition containing at least one antigen presenting cell (e.g., a dendritic cell) that is pulsed or loaded with one or more neoantigenic peptides or polynucleotides described herein. In some embodiments, such APCs are autologous (e.g., autologous dendritic cells). Alternatively, peripheral blood mononuclear cells (PBMCs) isolated from a patient can be loaded with neoantigenic peptides or polynucleotides ex vivo. In related embodiments, such APCs or PBMCs are injected back into the patient. In some embodiments, the antigen presenting cells are dendritic cells. In related embodiments, the dendritic cells are autologous dendritic cells that are pulsed with the neoantigenic peptide or nucleic acid. The neoantigenic peptide can be any suitable peptide that gives rise to an appropriate T cell response. T cell therapy using autologous dendritic cells pulsed with peptides from a tumor associated antigen is disclosed in Murphy et al. (1996) The Prostate 29, 371-380 and Tjua et al. (1997) The Prostate 32, 272-278. In some embodiments, the T cell is a CTL (e.g., CD8⁺). In some embodiments, the T cell is a helper T lymphocyte (Th (e.g., CD4⁺)).

In some embodiments, the present disclosure provides a composition comprising a cell-based immunogenic pharmaceutical composition that can also be administered to a subject. For example, an antigen presenting cell (APC) based immunogenic pharmaceutical composition can be formulated using any of the well-known techniques, carriers, and excipients as suitable and as understood in the art. APCs include monocytes, monocyte-derived cells, macrophages, and dendritic cells. Sometimes, an APC based immunogenic pharmaceutical composition can be a dendritic cell-based immunogenic pharmaceutical composition.

A dendritic cell-based immunogenic pharmaceutical composition can be prepared by any methods well known in the art. In some cases, dendritic cell-based immunogenic pharmaceutical compositions can be prepared through an ex vivo or in vivo method. The ex vivo method can comprise the use of autologous DCs pulsed ex vivo with the polypeptides described herein, to activate or load the DCs prior to administration into the patient. The in vivo method can comprise targeting specific DC receptors using antibodies coupled with the polypeptides described herein. The DC-based immunogenic pharmaceutical composition can further comprise DC activators such as TLR3, TLR-7-8, and CD40 agonists. The DC-based immunogenic pharmaceutical composition can further comprise adjuvants, and a pharmaceutically acceptable carrier.

Antigen presenting cells (APCs) can be prepared from a variety of sources, including human and non-human primates, other mammals, and vertebrates. In certain embodiments, APCs can be prepared from blood of a human or non-human vertebrate. APCs can also be isolated from an enriched population of leukocytes. Populations of leukocytes can be prepared by methods known to those skilled in the art. Such methods typically include collecting heparinized blood, apheresis or leukopheresis, preparation of buffy coats, rosetting, centrifugation, density gradient centrifugation (e.g., using Ficoll, colloidal silica particles, and sucrose), differential lysis non-leukocyte cells, and filtration. A leukocyte population can also be prepared by collecting blood from a subject, defibrillating to remove the platelets and lysing the red blood cells. The leukocyte population can optionally be enriched for monocytic dendritic cell precursors.

Blood cell populations can be obtained from a variety of subjects, according to the desired use of the enriched population of leukocytes. The subject can be a healthy subject. Alternatively, blood cells can be obtained from a subject in need of immunostimulation, such as, for example, a cancer patient or other patient for which immunostimulation will be beneficial. Likewise, blood cells can be obtained from a subject in need of immune suppression, such as, for example, a patient having an autoimmune disorder (e.g., rheumatoid arthritis, diabetes, lupus, multiple sclerosis, and the like). A population of leukocytes also can be obtained from an HLA-matched healthy individual.

When blood is used as a source of APC, blood leukocytes may be obtained using conventional methods that maintain their viability. According to one aspect of the present disclosure, blood can be diluted into medium that may or may not contain heparin or other suitable anticoagulant. The volume of blood to medium can be about 1 to 1. Cells can be concentrated by centrifugation of the blood in medium at about 1,000 rpm (150 g) at 4° C. Platelets and red blood cells can be depleted by resuspending the cells in any number of solutions known in the art that will lyse erythrocytes, for example ammonium chloride. For example, the mixture may be medium and ammonium chloride at about 1:1 by volume. Cells may be concentrated by centrifugation and washed in the desired solution until a population of leukocytes, substantially free of platelets and red blood cells, is obtained. Any isotonic solution commonly used in tissue culture may be used as the medium for separating blood leukocytes from platelets and red blood cells. Examples of such isotonic solutions can be phosphate buffered saline, Hanks balanced salt solution, and complete growth media. APCs and/or APC precursor cells may also purified by elutriation.

In some embodiments, the APCs can be non-nominal APCs under inflammatory or otherwise activated conditions. For example, non-nominal APCs can include epithelial cells stimulated with interferon-gamma, T cells, B cells, and/or monocytes activated by factors or conditions that induce APC activity. Such non-nominal APCs can be prepared according to methods known in the art.

The APCs can be cultured, expanded, differentiated and/or, matured, as desired, according to the according to the type of APC. The APCs can be cultured in any suitable culture vessel, such as, for example, culture plates, flasks, culture bags, and bioreactors.

In certain embodiments, APCs can be cultured in suitable culture or growth medium to maintain and/or expand the number of APCs in the preparation. The culture media can be selected according to the type of APC isolated. For example, mature APCs, such as mature dendritic cells, can be cultured in growth media suitable for their maintenance and expansion. The culture medium can be supplemented with amino acids, vitamins, antibiotics, divalent cations, and the like. In addition, cytokines, growth factors and/or hormones, can be included in the growth media. For example, for the maintenance and/or expansion of mature dendritic cells, cytokines, such as granulocyte/macrophage colony stimulating factor (GM-CSF) and/or interleukin 4 (IL-4), can be added. In other embodiments, immature APCs can be cultured and/or expanded. Immature dendritic cells can they retain the ability to uptake target mRNA and process new antigen. In some embodiments, immature dendritic cells can be cultured in media suitable for their maintenance and culture. The culture medium can be supplemented with amino acids, vitamins, antibiotics, divalent cations, and the like. In addition, cytokines, growth factors and/or hormones, can be included in the growth media.

Other immature APCs can similarly be cultured or expanded. Preparations of immature APCs can be matured to form mature APCs. Maturation of APCs can occur during or following exposure to the neoantigenic peptides. In certain embodiments, preparations of immature dendritic cells can be matured. Suitable maturation factors include, for example, cytokines TNF-α, bacterial products (e.g., BCG), and the like. In another aspect, isolated APC precursors can be used to prepare preparations of immature APCs. APC precursors can be cultured, differentiated, and/or matured. In certain embodiments, monocytic dendritic cell precursors can be cultured in the presence of suitable culture media supplemented with amino acids, vitamins, cytokines, and/or divalent cations, to promote differentiation of the monocytic dendritic cell precursors to immature dendritic cells. In some embodiments, the APC precursors are isolated from PBMCs. The PBMCs can be obtained from a donor, for example, a human donor, and can be used freshly or frozen for future usage. In some embodiments, the APC is prepared from one or more APC preparations. In some embodiments, the APC comprises an APC loaded with the first and second neoantigenic peptides comprising the first and second neoepitopes or polynucleotides encoding the first and second neoantigenic peptides comprising the first and second neoepitopes. In some embodiments, the APC is an autologous APC, an allogenic APC, or an artificial APC.

In an embodiment, the present disclosure provides a composition comprising an APC comprising a first peptide comprising a first neoepitope and a second peptide comprising a second neoepitope, wherein the first peptide is different from the second peptide, and wherein the first neoepitope comprises a mutation and the second neoepitope comprises the same mutation. In some embodiments, the first and second peptides are derived from the same protein. In another embodiment, the present disclosure provides a composition comprising an APC comprising a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same.

### Adjuvants

An adjuvant can be used to enhance the immune response (humoral and/or cellular) elicited in a patient receiving a composition as provided herein. The active ingredient in the composition comprise antigen peptides. Adjuvant incorporation can further enhance the antigenic response of the composition. Sometimes, adjuvants can elicit a Th1-type response. Other times, adjuvants can elicit a Th2-type response. A Th1-type response can be characterized by the production of cytokines such as IFN-γ as opposed to a Th2-type response which can be characterized by the production of cytokines such as IL-4, IL-5 and IL-10.

In some aspects, lipid-based adjuvants, such as MPLA and MDP, can be used with the immunogenic pharmaceutical compositions disclosed herein. Monophosphoryl lipid A (MPLA), for example, is an adjuvant that causes increased presentation of liposomal antigen to specific T Lymphocytes. In addition, a muramyl dipeptide (MDP) can also be used as a suitable adjuvant in conjunction with the immunogenic pharmaceutical formulations described herein.

Suitable adjuvants are known in the art (see, WO 2015/095811) and include, but are not limited to poly(I:C), poly-ICLC, Hiltonol, STING agonist, 1018 ISS, aluminum salts, Amplivax, AS15, BCG, CP-870,893, CpG7909, CyaA, dSLIM, GM-CSF, IC30, IC31, Imiquimod, ImuFact IMP321, IS Patch, ISS, ISCOMATRIX, JuvImmune, LipoVac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel®. vector system, PLG microparticles, resiquimod, SRL172, virosomes and other virus-like particles, YF-17D, VEGF trap, R848, beta-glucan, Pam3Cys, Pam3CSK4, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants also include incomplete Freund's or GM-CSF. Several immunological adjuvants (e.g., MF59) specific for dendritic cells and their preparation have been described previously (Dupuis M, et al., Cell Immunol. 1998; 186(1): 18-27; Allison A C; Dev. Biol. Stand. 1998; 92:3-11) (Mosca et al. Frontiers in Bioscience, 2007; 12:4050-4060) (Gamvrellis et al. Immunol & Cell Biol. 2004; 82: 506-516). Also, cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, PGE1, PGE2, IL-1, IL-1b, IL-4, IL-6 and CD40L) (U.S. Pat. No. 5,849,589 incorporated herein by reference in its entirety) and acting as immunoadjuvants (e.g., IL-12) (Gabrilovich D I, et al., J. Immunother. Emphasis Tumor Immunol. 1996 (6):414-418).

Adjuvant can also comprise stimulatory molecules such as cytokines. Non-limiting examples of cytokines include: CCL20, a-interferon (IFN-a), β-interferon (IFN-β), γ-interferon, platelet derived growth factor (PDGF), TNFα, TNFβ (lymphotoxin alpha (LTα)), GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, IL-28, MHC, CD80, CD86, IL-1, IL-2, IL-4, IL-5, IL-6, IL-10, IL-18, MCP-1, MIP-1a, MIP-1-, IL-8, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-1, DR3, TRAMP, Apo-3, AIR, LARD, NGRF, DR4, DRS, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-I, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAPI, and TAP2.

Additional adjuvants include: MCP-1, MIP-1a, MIP-1p, IL-8, RANTES, L-selectin, P-selectin, E-selectin, CD34, GlyCAM-1, MadCAM-1, LFA-1, VLA-1, Mac-1, pl50.95, PECAM, ICAM-1, ICAM-2, ICAM-3, CD2, LFA-3, M-CSF, G-CSF, IL-4, mutant forms of IL-18, CD40, CD40L, vascular growth factor, fibroblast growth factor, IL-7, IL-22, nerve growth factor, vascular endothelial growth factor, Fas, TNF receptor, Fit, Apo-1, p55, WSL-1, DR3, TRAMP, Apo-3, AIR, LARD, NGRF, DR4, DR5, KILLER, TRAIL-R2, TRICK2, DR6, Caspase ICE, Fos, c-jun, Sp-1, Ap-1, Ap-2, p38, p65Rel, MyD88, IRAK, TRAF6, IκB, Inactive NIK, SAP K, SAP-1, JNK, interferon response genes, NFκB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP1, TAP2 and functional fragments thereof.

In some aspects, an adjuvant can be a modulator of a toll like receptor. Examples of modulators of toll-like receptors include TLR-9 agonists and are not limited to small molecule modulators of toll-like receptors such as Imiquimod. Other examples of adjuvants that are used in combination with an immunogenic pharmaceutical composition described herein can include and are not limited to saponin, CpG ODN and the like. Sometimes, an adjuvant is selected from bacteria toxoids, polyoxypropylene-polyoxyethylene block polymers, aluminum salts, liposomes, CpG polymers, oil-in-water emulsions, or a combination thereof. Sometimes, an adjuvant is an oil-in-water emulsion. The oil-in-water emulsion can include at least one oil and at least one surfactant, with the oil(s) and surfactant(s) being biodegradable (metabolisable) and biocompatible. The oil droplets in the emulsion can be less than 5 μm in diameter, and can even have a sub-micron diameter, with these small sizes being achieved with a microfluidiser to provide stable emulsions. Droplets with a size less than 220 nm can be subjected to filter sterilization.

Methods for Generating Polybodies with Epitope Peptides

Polybodies can be generated by cloning. In one approach, a parent scaffold assembly comprising the antigen peptides is first generated. The parent scaffold can be digested with restriction enzymes, following which, a peptide or a polypeptide comprising the epitope is cloned into the restriction sites to obtain the peptide-scaffold unit(s) which are then inserted in a linearized expression vector. The peptides may comprise flanking linker sequences. Alternatively, peptide sequences with linkers can synthesized; or can be generated by PCR. For ease of joining the vector with the insert (the peptide or a polypeptide to be inserted in the vector) Gibson assembly can be utilized. Gibson cloning method offers fast and flexible cloning strategy, where any fragment can be joined without having specific restriction sites, and is regardless of the sequence. It involves a T5 exonuclease chew-back generating a single stranded overhangs at the 5′- and 3′-ends of the DNA fragments (in the insert), whereas the vector usually has staggered ends. The insert overhang thus created hybridizes with the vector's staggered ends upon contact, and fill-in ligation is performed using a suitable ligating enzyme. In some instances, larger overhangs may be generated, and the overhang regions of the vector and the insert join by hybridizing of the overhangs and are then ligated using suitable ligating enzyme to form the intact vector. Variations from the above described scheme are conceivable by one of skill in the art and are contemplated within the scope of the disclosure.

### Artificial Mini-Proteome Vaccine

Aggressive tumor eradication immunotherapy requires therapeutic products that are efficacious, can be rapidly generated and broadly adopted. Personalized medicine using a subject's own neoantigen peptides to activate T cells is a new and promising area in immune-oncology, with the caveat that preparing neoantigen vaccines may take days, to weeks or even months. Additionally, the methods are expensive. A more efficacious and time and cost effective method is hereby sought. The objective is to identify and develop rapid and inexpensive vaccine comprising most neoantigens and also other potentially useful antigens from the subject in a short span of time. Such a product should readily integrate with any off-the-shelf combinations of approved and developing immunotherapies; should be inexpensive to repeat preparations from additional tumor sample post-relapse or to reduce impact of inter-tumoral heterogeneity; should incorporate a significantly broader representation of potentially useful epitopes, should activate both CD8+ and CD4+ cells of the subject, among others. Provided herein are compositions and a method for vaccine production that satisfy the specific unmet needs as described above.

In one aspect, provided herein is a highly efficient method for generating a subject-specific cancer vaccine, which bypasses the steps of neoantigen prediction. The method relates to the generation of artificial mini-proteome vaccine (AmpVax). In this method, a personalized cancer vaccine can be generated by preparing a library of whole exome-capture RNA fragments from tumor cells or Fresh Frozen Paraffin Embedded (FFPE) tissue slices from a subject that encode the entire or a selected fraction of proteins expressed by the tumor cells, including all or almost all neoantigens as well as tumor associated antigens and additional regions near to introns and the untranslated regions, which might contain neoantigen translation products not readily determinable by nucleic acid sequencing and use of an antigen prediction algorithm. cDNA is prepared from the library of whole exome-capture RNA fragments. Each of the cDNA products from RNA fragments can be cloned in a suitable expression vector, and expressed in a suitable system, such as a bacterial system, (e.g., E. coli expression system). The expressed peptides can then be isolated, which can be used as a vaccine. In some embodiments, the isolated peptides expressed from the library of whole exome-capture RNA of a subject having a disease is the artificial mini-proteome, wherein the exome capture RNA is from a diseased cell of the subject. The expressed peptides can then be purified and can be used as vaccines without further modification.

In some embodiments, the polynucleotides encoding expressed peptides from the library of whole exome-capture RNA fragments (or, in other words, the whole exome cDNA library) are isolated, and sub-cloned upstream of a protein(s) or protein fragment(s) (e.g. scaffold proteins as described elsewhere in the specification) to generate peptide fusion constructs, such as polybodies, and a peptide fusion library is generated, that can be readily expressed in a rapid biological expression system such as E coli system at high yield. The expressed proteins from the peptide fusion library can be isolated, purified and used in its entirety as the artificial mini-proteome (Amp Vax) vaccine.

In one aspect, the exome capture RNA of the subject is further enriched for differentially expressed RNA in the diseased cell compared to the non-diseased cell of the subject or a standard non-diseased reference cell. Cancer cells accumulate somatic mutations in one or more genes, and such mutations are absent in the non-cancer cells of the subject, or comparable cells from a healthy individual. In some embodiments, RNA transcribed from the mutated genes in a cancer cell are distinct in nucleic acid sequence when compared to a non-cancer or healthy cell of the subject, or a non-cancer reference cell. Such RNA from the mutated genes can be considered as the differentially expressed genes in comparison to the reference. The differentially expressed RNA comprises, for example, a mutated gene product, such as a point mutation, or an insertion or deletion of one or more nucleotides. The differentially expressed RNA comprises, for example, RNA enriched for neoantigen encoding sequences. In some embodiments, the RNA that is expressed differentially in a diseased cell or tissue (e.g a cancer cell or a tumor) over a reference (e.g., RNA that is expressed by a non-diseased cell or tissue) is selected for use as the Amp Vax vaccine.

In essence, the technique allows for pan-exome neoantigen capture and enrichment.

The differentially expressed RNA described above can be obtained by techniques for enriching differentially expressed genes, e.g., techniques involving hybridization of the cancer cell-derived RNA with the reference RNA followed by isolation of the non-hybridized/mismatched regions. An exemplary method includes Mut S mediated isolation of mismatched DNA dimer.

In some embodiments, no sequencing is necessary for the vaccine generation process.

The composition of the library thus represents the content of expressed proteins within the tumor cell fused to carrier proteins for efficient production and antigen delivery.

The expressed protein(s) or protein fragments can be designed to be (1) large in size (>˜50-60 kD) which favors good lymph node retention; (2) to be modular and allow incorporation of functional domains (e.g., incorporating CLEC9A; such as for epitope cross-presenting and targeting dendritic cells, especially desired subsets of dendritic cells,) as well as (3) to be multi-valent (e.g., —contain multiple copies of a peptide sequence (e.g., a neoantigen or functional domain or scaffold domain) to allow further functionality addition, with multiple copies of the added component per molecule, by simple add-mix based functionalization with anti-scaffold epitope directed antibodies or antibody fragments carrying adjuvant, DC-targeting or other vaccine functionality enhancing groups. For example, the vaccine product is expressed in E coli and purified using conventional affinity technology, ion exchange and/or size exclusion chromatography steps. The vaccine can be prepared without the need for sequencing or bioinformatics and without the need for synthesis of multiple defined sequence oligonucleotides, and hence can be rapidly prepared.

In some embodiments, a biological sample is obtained from a subject having a disease, e.g. cancer; the biological sample can be any one or more of: a tumor biopsy, cells from an apheresis column, cells from peripheral blood, paraffin embedded tissue slices, or frozen paraffin embedded tissue samples. In some embodiments, total cellular RNA is obtained from a cell of the subject. In some embodiment, total cellular RNA is obtained from a diseased cell of the subject, wherein the diseased cell is a cancer cell, or an infected cell. In some embodiments, total cellular RNA is obtained from both a diseased cell (e.g., a cancer cell) from the subject and a non-diseased (e.g., non-cancer cell) of the subject, that can be used for enriching differentially expressed RNA in the diseased cell with reference to the non-diseased cell. In some embodiments.

One advantage of the method described herein is that the vaccine covers broad representation of potential antigens.

Another advantage of the method described herein is that it facilitates epitope spread.

Another advantage of the method described herein is that it is safe from toxicity or side effects.

Another advantage of the method described herein is that it is relatively fast. In some embodiments, the vaccine is prepared in less than 2 weeks from obtaining the biological sample that comprises the total cellular RNA. In some embodiments, the vaccine is prepared in less than one week. In some embodiments the vaccine prepared in less than 12 days, less than 10 days, less than 9 days, less than 8 days, less than 7 days, less than 6 days, less than 5 days, less than 4 days or less than 3 days. An exemplary simplified workflow of the method is given below, which shows the approximate time required for preparation of the vaccine from the isolation of cells from the subject:
(a) Whole exome-capture RNA isolation and cDNA library preparation: 2 days
(b) Cloning in plasmid: 1 day
(c) Protein expression: expansion 4-5 days
(d) Purification and vaccine composition: 1-2 days; therefore, the process can complete in approximately 10 days. The procedure can be expedited with optimization and further improvements in the protocol.

Cloning and expression of the cDNA library from exome capture RNA can be carried on by using an in-house generated and optimized protocol and expression system. In an exemplary protocol, briefly, total RNA is extracted from a biological sample of the subject. The biological sample may comprise cells from the cancer or tumor of the subject. In some embodiments the biological sample may comprise a first biological sample, comprising cells or tissue (fresh or fixed, preserved) from a cancer or tumor of the subject; and a second biological sample, comprising cells or tissue (fresh or fixed, preserved) from a non-diseased cell, such as a non-tumor cell of the subject. RNA extracted from the biological sample can be mechanically, chemically or enzymatically digested into fragments that are about 24 to about 300 nucleotide bases in length, or about 45-450 nucleotide bases in length; or 100-300 nucleotide bases in length; or about 250 bases in length. Some preserved samples may have naturally degraded or sheared RNA. cDNA may be prepared from the total RNA with reverse transcriptase, using random primers. In some embodiments, a step can be employed to capture the coding regions of the transcriptome, e.g., an RNA or cDNA exome capture step. For example, RNA or cDNA libraries can be hybridized to exon-specific DNA or RNA baits, which are in turn biotinylated. Following hybridization, the biotinylated DNA or RNA baits can be captured with streptavidin-coated magnetic beads. RNA or cDNA fragments bound to the exon-specific baits can be pulled down and unbound RNA or cDNA fragments can be washed away. The remaining captured RNA or cDNA fragments can then be amplified, for example after being denatured from the capture probes. The cDNA fragments generated can next be cloned into an expression vector for generating the whole exome capture cDNA library. In some embodiments, the cDNA library is subjected to denaturation, optionally ligated to strand specific adapters; hybridized with a reference exome library or genomic library, and subjected to enrichment of mutation containing DNA fragments by passing through columns comprising immobilized MutS protein that can bind to DNA duplex having at least one nucleotide mismatch. In some embodiments, the hybridized exome/reference library can be subjected to enrichment of mutation containing DNA fragments by interacting with MutS in solution followed by capture of MutS on a column. The expression plasmid comprises (a) a promoter sequence, (b) a sequence comprising the polynucleotide fragment (in this case a cDNA fragment from the cDNA library), and (c) ideally, at least an affinity tag and/or a tag that allows identification of an expressed peptide from the cloned cDNA sequence fragment. Exemplary affinity tags are: a peptide sequence that binds to an Fc receptor, a GST-tag, a His-tag, a peptide sequence that binds to Protein A, a peptide sequence that binds to Protein G or an epitope of an antibody or binding fragment thereof. In some embodiments a tag for ready identification of expressed peptide sequences can be included. Exemplary tags for the purpose include, apart from affinity tags described above which should also contribute to the purpose, an HA tag, a FLAG tag, a His6-tag, a fluorescent tag, such as GFP, RFP or YFP. An affinity tag may also be a region of a human scaffold protein that is expressed downstream of the polynucleotide fragment.

An affinity tag may be used to isolate and purify the peptide translated when the expression vector is expressed in a suitable cell, such as a bacterial cell expression system.

cDNA library cloning can be performed using standard methods. In addition, several modifications can be included to tailor the process for peptide generation. Prior to cloning the RT enzyme system uses the cDNA as the first strand (or template strand) to generate the duplicate strand, the resultant double stranded DNA is then cloned in a vector. In general, a single RT reaction in a reaction tube generates the insert double stranded DNA comprising the cDNA for cloning. Briefly the insert is ligated to the expression vector, downstream of a promoter sequence, and the vector may comprise a start codon at or near the 3′-end of the positive strand. The insert may be ligated immediately downstream of the start codon, or 1, 2, 3, or more nucleotides downstream of the start codon. In some embodiments, the insert may be ligated to the vector via a linker sequence.

In some embodiments the cDNA fragment may be fused to a linker at the 5′ end, at the 3′ end or at both the 5′ and the 3′ end. A linker is a short polynucleotide sequence. In some embodiments a linker may encode a polypeptide or an oligopeptide. The linker may be configured to facilitate a cleavage of the linker, that is, the linker comprises a cleavage sequence. In some embodiments, the cleavage sequence may be a proteolytic cleavage sequence. The cleavage may be a post-translational cleavage event. In some embodiments, the linker polynucleotide may comprise a restriction digestion site for digesting the nucleic acid and facilitate cloning.

At the 3′end, the insert may be ligated to a sequence comprising a frame check sequence. In some embodiments, the 3′end of the insert may be ligated to a sequence encoding one or more affinity tags downstream of the frame check sequence. In some embodiments the frame check sequence comprises a sequence encoding an affinity tag. An exemplary frame check sequence encodes Val-Gly-Ser.

In some embodiments, the frame check sequence has a formula of N₁-N₂-N₃-N₄-N₅-N₆-N₇-N₈-N₉: wherein, each N is independently a nucleotide selected from the group consisting of A, T, U, C and G; each of N₁-N₂-N₃ and N₄-N₅-N₆ and N₇-N₈-N₉ is not a stop codon; and each of N₂-N₃-N₄ and N₆-N₇-N₈ is a stop codon.

Exemplary cloning techniques include but are not limited to: TA cloning, Topo PCR cloning, blunt-end cloning, gateway cloning, and others.

In some embodiments, the cDNA fragments, and 5′- and/or 3′-linkers if present, are pre-ligated together or generated as a PCR products. In some embodiments the insert comprising the cDNA fragments; or the cDNA fragments with the linkers as a whole are then ligated to a polynucleotide sequence encoding scaffold proteins; such that at least one scaffold protein is in the same reading frame with at least one cDNA fragment. In some embodiments, parent scaffold protein sequences may be digested by restriction enzymes to ligate the insert, wherein the insert also comprises the restriction digestion sites in the flanking regions comprising the linker sequences. In some embodiments, a plurality of such cDNA sequences, flanked by a linkers, and comprising one or more scaffold peptide sequences on either side of each DNA can constitute the insert for cloning and expression in a suitable expression system.

A suitable expression system can be a bacterial expression system. An example is the E. coli expression system, such as the pTC7 vector systems. However, other expression systems may also be contemplated. Examples include mammalian cell based expression system, yeast-based expression systems, insect cell based expression systems, or cell free expression systems.

In some embodiments, the baculovirus expression system (BAC) is used.

In some embodiments a phage expression system is used.

As is well understood by one of skill in the art, the cloning and expression allows expression of one vector to be expressed in a cell, such as an E. coli. In some embodiments, expression of an insert is first detected before incorporating the cDNA fragment in a scaffold cassette. In some embodiment, a small scale analysis is done for peptide expressing fragments before large scale manufacture of the vaccine from a subject. In some embodiments, the purification step isolates only the expressing cDNA fragments, which is an easy proof of expression from the cDNA insert. A variety of situations may arise from cloning whole exome capture cDNA library. For example, (i) some cloned cDNA fragments may enter in-frame, and exit in-frame, and both the peptide and the affinity tag are expressed; (ii) some cloned cDNA fragments may enter in-frame, but exit out of frame, in such cases, the peptide is expressed but the affinity tag is not expressed; (iii) some cloned cDNA fragments may enter in-frame, and exit in-frame, and a partial peptide is expressed but the affinity tag is not expressed; (iv) some cloned cDNA fragments may enter out of frame, and exit in-frame, and no peptide is expressed but the affinity tag is expressed; or, (v) some cloned cDNA fragments may enter out of frame, and exit out of frame, in such case, neither a peptide nor the affinity tag is expressed.

In some embodiments, the workflow includes first identification of a peptide at least by expression of the affinity tag, or isolation of the affinity purified tags, following which, a fraction of the purified product may then be archived for future use, such as identifying and expressing more clones of the same from the library; while the rest is used for generation of the peptide vaccines.

The expression system can be optimized for scale up as necessary.

In some embodiments, the Amp Vax whole exome (Amp Vax-exome) vaccine is generated. The vaccine generated by this method comprises the whole exome RNA-generated cDNA in the vaccine, as described above or a selected portion of the whole exome expected to be expressed in the tumor cells based on the tumor type. In some embodiments the Amp Vax-enriched exome vaccine is generated. The vaccine generated by this method comprises cDNA enriched for expression in the diseased cells of the subject compared to a reference exome, as described in the section below. In an exemplary scheme to describe both the processes, first, the whole exome capture RNA is fragmented and double stranded cDNA ligated library, where each cDNA fragment is ligated with strand-specific primers is prepared in both the methods. The libraries are then subjected to bait hybridization, where in case of the Amp Vax-exome procedure, the exomes are captured as such; whereas in case of the AmpVax-enriched exome vaccine generation, heteroduplexes are subjected to MutS enrichment, followed by an exome capture step. The MutS enrichment selectively enriches for mismatches in the double strand, therefore enriching the mutations in the cDNA sequences. The whole exome cDNA or the MutS captured enriched cDNA are then cloned into a plasmid.

Method for exome analysis and enrichment of polynucleotides comprising point-mutations: Exome can be defined as all genes in the genome that are expressed in an organism. Generally, the pool of messenger RNA reflects all genes that are expressed in a particular cell or tissue of the organism at a particular time and set of conditions that the cell or tissue was experiencing. In a human being, there are about 180,000 exons, which constitute about 1% of the human genome, or approximately 30 million base pairs. Total RNA extracted from a cell comprises the total messenger RNA repository, which can be converted to cDNA in vitro using a suitable reverse transcriptase (RT) enzyme, suitable primers, and the necessary reagents, such as nucleotides (dNTPs), salt and buffer in a nuclease free solution. In some embodiments the Linkers or adapters are then attached to the cDNA fragments, which are then hybridized to a library of polynucleotides designed to capture only the exons. The hybridized DNA fragments can then selectively isolated and cloned into an expression vector. Selective isolation of DNA fragments that comprise a mutation, such as a point mutation can be achieved by subjecting the hybridized DNA to be in contact with MutS proteins, as described earlier, and captured using a MutS column. Mutations that include a mismatch of 1 or few nucleotides (e.g., less than five) in the hybridized DNA can be isolated using MutS separation.

A “reference genome” is a defined genome used as a basis for genome comparison or for alignment of sequencing reads thereto. A reference genome may be a subset of or the entirety of a specified genome; for example, a subset of a genome sequence, such as exome sequence, or the complete genome sequence.

Whole exome analysis can be done from total RNA extracted from a tissue of a subject. A biological tissue sample obtained from the subject can be preserved in various forms for future analysis, for example, in the form of fresh tissue isolate, total RNA extracted and stored from fresh tissue, fresh flash frozen tissue, or formalin fixed paraffin embedded tissue (FFPE), the latter being the most common form of sample as which is suitable for histological analysis as well as RNA extraction. However, one of skill in the art is aware and appreciates that although FFPE samples are easy to preserve and obtain, it is difficult to extract good quality mRNA from FFPE samples. In most cases, chaotropic solutions are required to remove the paraffin and extract the RNA. RNA can be recovered from the chaotropic solution by extraction with an organic solvent, chloroform extraction, phenol-chloroform extraction, precipitation with ethanol, isopropanol or any other lower alcohol, by chromatography including ion exchange chromatography, size exclusion chromatography, silica gel chromatography and reversed phase chromatography, or by electrophoretic methods, including polyacrylamide gel electrophoresis and agarose gel electrophoresis, as will be apparent to one of skill in the art. RNA is preferably recovered from the chaotropic solution using phenol chloroform extraction. Following RNA recovery, the RNA may optionally by further purified. Further purification results in RNA that is substantially free from contaminating DNA or proteins. Further purification may be accomplished by any of the aforementioned techniques for RNA recovery. RNA is preferably purified by precipitation using a lower alcohol, especially with ethanol or with isopropanol. Precipitation is preferably carried out in the presence of a carrier such as glycogen that facilitates precipitation.

In brief, RNA can be isolated from paraffin embedded tissue as follows. A single 5 μm section of paraffinized tissue is placed in an Eppendorf tube and deparaffinized by two washes with xylene. The tissue is rehydrated by successive washes with graded alcohols (100%, 95%, 80% and 70%). The resulting pellet is suspended in 4M guanidine isothiocyanate with 0.5% sarcosine and 20 mM dithiothreitol (DTT). The suspension is homogenized and then heated to from about 50 to about 95° C. for 0 to 60 minutes; a zero-heating time-point, is included as a control for each sample. Sodium acetate (pH 4.0) is added to 0.2 M and the solution is extracted with phenol/chloroform and precipitated with isopropanol and 10 mg glycogen. After centrifugation (13000 rpm, 4° C., 15 min) the RNA pellet is washed twice with 1 mL of 75% ethanol then resuspended in RNase-free water.

Total RNA was converted to cDNA using random hexamers. RT conditions were as have been previously described for frozen tissue (Horikoshi et al., 1992).

A subject having a cancer generally exhibits a large number of mutations throughout the genome. The mutations can be point mutations, which are the most abundant and can account for a large number of altered peptide or proteins in the cancer genome. Other mutations can be insertions or deletions of one or more nucleotides at a stretch, frame shift mutations which impact longer regions of a gene, generation of neo-ORFs by strand breakage and rejoining, among others. In the method described herein, comparison of exomes of a cancer cell or tissue of a subject with a reference exome, is best suited to capture a large number of point mutations within the exome of the cancer cell.

In some embodiments, a cDNA sample is prepared from a cancer tissue sample of a subject with cancer for exome analysis. In some embodiments, cDNA from a cancer sample is compared with a reference cDNA sample. In some embodiments, the reference sample represents a reference exome probe. In some embodiments, the reference cDNA is prepared from a non-cancer cell or tissue from the subject.

In some embodiments, the comparison of the cancer sample exome with the reference exome is performed via a hybridization step. In some embodiments, a polynucleotide comprising an enriched set of polynucleotide sequences is generated, wherein the enriched set of polynucleotide sequences comprises a whole exome set of point-mutant containing polynucleotides, which are enriched from a tumor sample of a subject with cancer; and wherein each of the point-mutant containing polynucleotides are hybridized to a reference polynucleotide with less than 100% sequence complementarity to the corresponding point-mutant containing polynucleotide to which it is hybridized. In some embodiments, a hybridization step is followed by a cloning of the specific exome set from the cancer sample of the subject in an expression cloning vector, thereby creating a library of cancer sample exome that carries point mutations in the cancer exome, relative to the reference exome. Various selective hybridization and cloning procedures are contemplated here. These include subtractive hybridization, suppressed subtractive hybridization, selective hybridization and such methods are known to one of skill in the art.

Contemplated herein is a method of using strand specific adaptors for cloning the cancer exome into an expression vector. In some embodiments, a first strand specific adaptor is ligated to the cDNA fragments from the cancer exome, wherein a second strand specific adaptor is ligated to the cDNA fragments from the reference exome, wherein the first strand specific adaptor is designated for ligation to the positive strand of the vector the for expression. In some embodiments, the first strand specific adaptor is such that when the first strand specific adaptor with the ligated cDNA is ligated to the vector, the cDNA strand is placed downstream of a promoter and other regulatory elements that initiate and control expression, whereas the second strand specific adaptor ligated to the reference exome is ligated to the strand of the vector that is not expressed. In some embodiments, each of the point-mutation containing polynucleotides comprises an adapter on a 5′ end of the point-mutation containing polynucleotide or a 3′ end of the point-mutation containing polynucleotide, and wherein each of the reference polynucleotides comprises an adapter on a 5′ end of the reference polynucleotide or an adapter on a 3′ end of the reference polynucleotide.

In one aspect, a short and efficient method for isolation and cloning of mutation enriched DNA from a cancer cell of a subject is contemplated and disclosed herein. The method comprises preparing a nucleic acid from a biological sample of the subject; enriching polynucleotides encoding cancer mutations, and therefore enriching sequences encoding neoepitopes; cloning the enriched polynucleotides; expressing the cloned polynucleotides to generate peptides (for example, by expressing the cloned polynucleotides in a bacterial expressed system) that can be used for therapeutic purpose. Peptides translated from cloned polynucleotides enriched in sequences encoding cancer mutations, harbor neoepitopes useful in cancer immunotherapy. The expressed peptides can be used as such as immunotherapy vaccines to activate an immune response in the subject against the cancer or tumor.

In some embodiments, the method described herein comprises the following advantages;
(i) The method comprises generation of pan-exome mutations that have wide neoepitope coverage for high effectiveness as cancer immunotherapy vaccine; and the vaccine is capable of activating both CD4+ and CD8⁺ T cells.
(ii) The method is simple and has fewer procedural steps than commonly used methods.
(iii) The method is directed to reducing time from isolation of a biological sample comprising tumor cells from a subject, to preparation of a therapeutic component.
(iv) The method is directed to reducing (preferably eliminating) the step of identifying neoepitope-encoding sequences, but at the same time enriching potential neoepitope encoding sequences.
(v) The method is cost efficient.

In some embodiments, the method encompasses a step of: (a) hybridizing a cancer cell exome-derived DNA fragments with a reference DNA that spans a representative human genome;
(b) isolating mismatched DNA fragments (that is, DNA fragments comprising a mutation, e.g. a point mutation) in a mismatch DNA capture step utilizing MutS columns, yielding DNA fragments enriched in cancer mutations;
(c) eluting and cloning the DNA fragments captured by the MutS columns;
(d) expressing the cloned DNA in a bacterial cloning and expression system, and purifying the protein products.

In some embodiments the exome derived DNA is cDNA. In some embodiments the exome derived DNA (or cDNA) is processed into fragments that are about 200 nucleotides long. In some embodiments, the exome derived DNA is obtained from RNA isolated from FFPE samples. In some embodiments, the method includes hybridization of strand specific adapter ligated cancer exome with the strand specific adapter ligated reference exome. The hybridized heterodimer polynucleotides will contain at least one nucleotide mismatch corresponding to each mutation in the cancer exome. In order to reduce enrichment of naturally occurring polymorphisms that are not cancer related, the reference genome or exome is carefully chosen. In some embodiments, the reference exome is optimized for preventing enrichment of naturally occurring polymorphisms.

Next, an agent, such as a mismatch specific protein can be used to bind to and identify the polynucleotides which comprise at least one mismatched nucleotide. Therefore, one or more of the point-mutant containing polynucleotides are bound to an agent that specifically binds to double stranded polynucleotides containing a mismatch. In some embodiments, the agent is DNA mismatch repair enzyme. In some embodiments, the agent is a MutS protein. MutS (Mutator S) protein plays an important role in the DNA repair system in prokaryotic and eukaryotic cells. MutS protein is an E. coli protein that is a part of the well-studied MutHLS pathway of mismatch recognition and repair (MMR), and homologs of the MutS family proteins are found in many species including eukaryotes. MutS proteins are involved in the first step in recognition and repair of mismatched DNA during replication. MutS proteins can be utilized as effective means to identify and capture hybridized DNA products having a DNA mismatch. The mismatch binding protein MutS can specifically recognize and bind to all possible single-base mismatches, as well as 1˜5 bases insertion or deletion loops, with varying affinities and functions independently of other proteins or cofactors. In the process described herein, a DNA mismatch in the hybridized DNA fragments represents a mutation in the cancer exome derived DNA. The MutS protein recognizes unpaired and mispaired bases in duplex DNA and can be used for detection of point mutations in vitro. The N-terminal domain of MutS is responsible for mismatch recognition and forms a 6-stranded mixed beta sheet surrounded by two alpha helices. MutS bound heterodimers could then be captured in a MutS binding column and eluted to obtain the cancer exome comprising point mutations. These polynucleotides constitute an enriched set of polynucleotides, for the preparation of the vaccine. These polynucleotides are cloned for selected expression of the cancer exome strands as described above, to obtain the exome library comprising the differentially expressed genes in the cancer tissue or cell of the subject, relative to the suitable non-cancer control.

In some embodiments the enrichment is done with target specific probes. Probes can be specific nucleic acid sequences, which can be conjugated to a tag that enables separation and harvesting of the enriched polynucleotides. The tag may be an affinity tag, for example, biotin, His, HA and others. Separation may be done by passing the probe-bound and unbound hybridized DNA through an immobilized probe capture bed, such as an affinity column, where the hybridized probes are captured by components that bind to the tag.

In some embodiments, the polynucleotides thus enriched are further modified, for example ligated to polybodies.

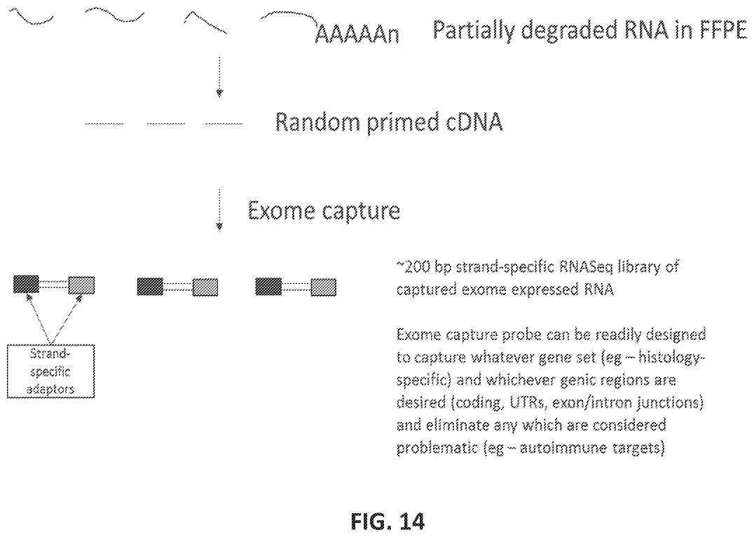

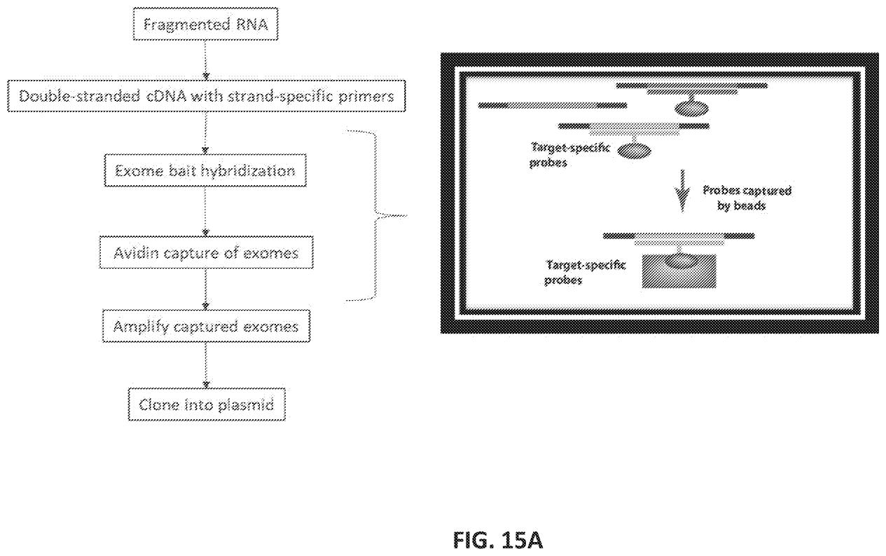

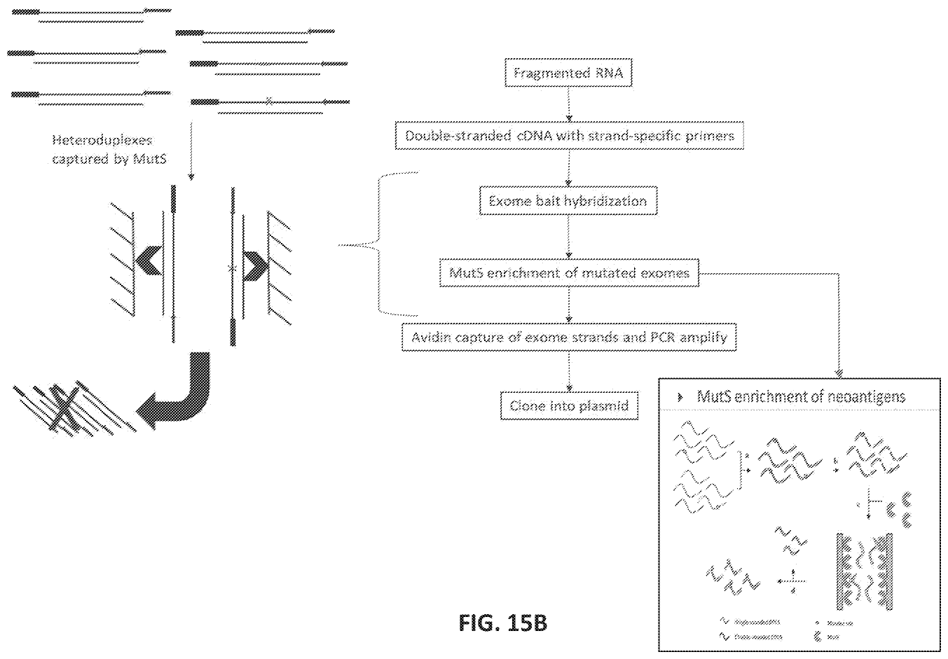

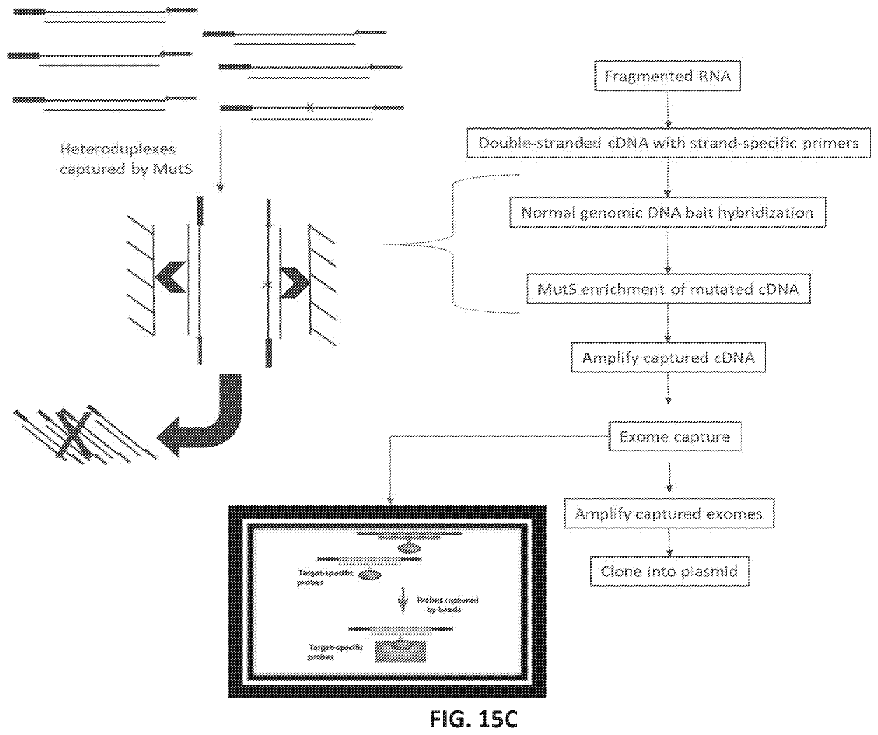

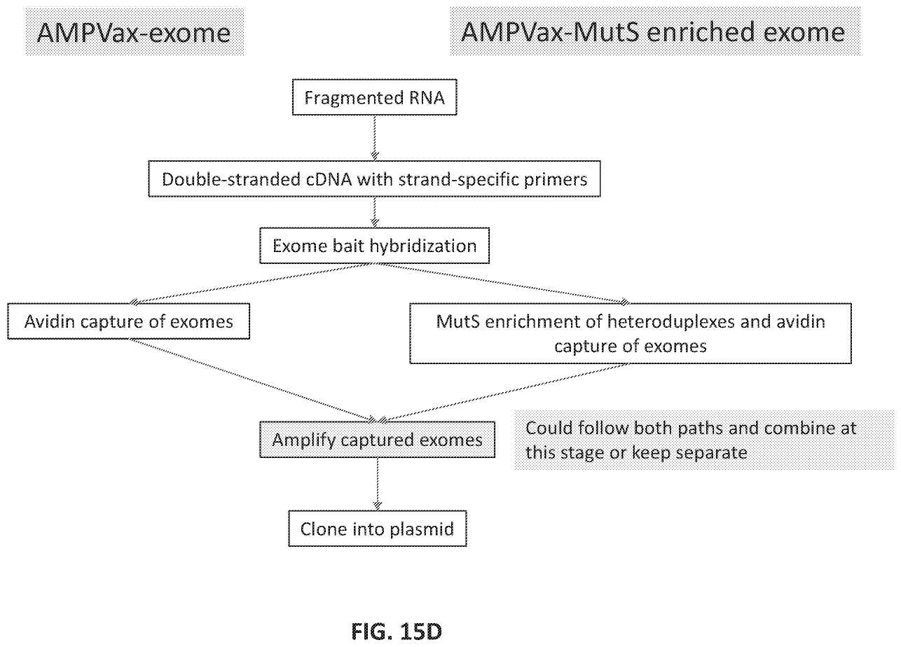

In some embodiments the enriched polynucleotides comprising the mutations in the subject's cancer exome are further modified, for cell-specific targeting. In some embodiments the enriched polynucleotides comprising the mutations in the subject's cancer exome are ligated to polynucleotides encoding one or more polypeptides having effector functions, for example, increasing immune response of the translated products. In some embodiments the enriched polynucleotides can be ligated to polybodies having specialized functions as described earlier. Representative method steps are delineated as diagrammatic representations of FIGS. 14, 15A, 15B, 15C and 15D.

In some embodiments vectors are designed to comprise polynucleotides encoding scaffold proteins comprised in a polybody co-transcribed with the enriched polynucleotide. In some embodiments, the polynucleotide sequence encoding the scaffold protein may further be modified for functional enhancements.

In some embodiments, the MutS enriched polynucleotide fragments are cloned and ligated to polybodies that have specific targeting mechanisms, as described in the preceding sections. In some embodiments, the polybodies comprise mechanisms of targeting to dendritic cells (DCs). The targeting mechanism can target Clec9A protein specifically on the DCs. In some embodiments, the vaccine thus prepared from expressing the MutS enriched polynucleotide fragments, and further comprising polybodies as described herein can be targeted for lymph node accumulation.

### Methods of Treatment and Pharmaceutical Compositions

The neoantigen therapeutics described herein are useful in a variety of applications including, but not limited to, therapeutic treatment methods, such as the treatment of cancer. In some embodiments, the therapeutic treatment methods comprise immunotherapy. In certain embodiments, a neoantigenic peptide is useful for activating, promoting, increasing, and/or enhancing an immune response, redirecting an existing immune response to a new target, increasing the immunogenicity of a tumor, inhibiting tumor growth, reducing tumor volume, increasing tumor cell apoptosis, and/or reducing the tumorigenicity of a tumor. The methods of use can be in vitro, ex vivo, or in vivo methods.

In some aspects, provided herein is a method of treatment comprising providing a plurality of distinct template polynucleotides from a sample comprising diseased cells from a subject with a disease; attaching adaptor sequences to the plurality of distinct template polynucleotides; thereby forming a plurality of distinct adaptor tagged template polynucleotides; amplifying the distinct adaptor tagged template polynucleotides; inserting the amplified distinct adaptor tagged template polynucleotides into a vector, thereby forming a library of recombinant expression constructs; expressing polypeptides encoded by the library of recombinant expression constructs; enriching for the expressed polypeptides; and administering the enriched expressed polypeptides to the subject.

In some aspects, the present disclosure provides methods for activating an immune response in a subject using a neoantigenic peptide or protein described herein. In some embodiments, the present disclosure provides methods for promoting an immune response in a subject using a neoantigenic peptide described herein. In some embodiments, the present disclosure provides methods for increasing an immune response in a subject using a neoantigenic peptide described herein. In some embodiments, the present disclosure provides methods for enhancing an immune response using a neoantigenic peptide. In some embodiments, the activating, promoting, increasing, and/or enhancing of an immune response comprises increasing cell-mediated immunity. In some embodiments, the activating, promoting, increasing, and/or enhancing of an immune response comprises increasing T cell activity or humoral immunity. In some embodiments, the activating, promoting, increasing, and/or enhancing of an immune response comprises increasing CTL or Th activity. In some embodiments, the activating, promoting, increasing, and/or enhancing of an immune response comprises increasing NK cell activity. In some embodiments, the activating, promoting, increasing, and/or enhancing of an immune response comprises increasing T cell activity and increasing NK cell activity. In some embodiments, the activating, promoting, increasing, and/or enhancing of an immune response comprises inhibiting or decreasing the suppressive activity of T regulatory (Treg) cells. In some embodiments, the immune response is a result of antigenic stimulation.

In some embodiments, the present disclosure provides methods of activating, promoting, increasing, and/or enhancing of an immune response using a polybody comprising a neoantigenic peptide described herein. In some embodiments, a method comprises administering to a subject in need thereof a therapeutically effective amount of a polybody comprising a neoantigenic peptide that delivers a neoantigenic peptide to a tumor cell. In some embodiments, a method comprises administering to a subject in need thereof a therapeutically effective amount of a polybody comprising a neoantigenic peptide internalized by an antigen presenting cell, such as a tumor cell. In some embodiments, a method comprises administering to a subject in need thereof a therapeutically effective amount of a vaccine comprising a polybody comprising neoantigenic peptides that are internalized by an antigen presenting cell, and the neoantigenic peptides are processed by the cell.

In some embodiments, the method comprises administering to a subject in need thereof a therapeutically effective amount of a vaccine comprising peptides enriched in neoantigenic peptide or polynucleotide described herein that delivers an exogenous polypeptide comprising at least one neoantigenic peptide to the subject, wherein at least one neoepitope derived from the neoantigenic peptide is presented on the surface of the tumor cell in complex with a MHC class I molecule. In some embodiments, the neoepitope is presented on the surface of the tumor cell in complex with an MHC class II molecule. In some embodiments the method results in induction of, or enhancement of the immune response of the subject to the at least one neoepitope derived from the neoantigenic peptide.

In some embodiments, a method comprises contacting a tumor cell with a polybody comprising a neoantigenic polypeptide described herein that delivers an exogenous polypeptide comprising at least one neoantigenic peptide to the tumor cell, wherein at least one neoepitope derived from the polybody is presented on the surface of the tumor cell. In some embodiments, the neoepitope is presented on the surface of the tumor cell in complex with an MHC class I molecule. In some embodiments, the neoepitope is presented on the surface of the tumor cell in complex with an MHC class II molecule.

In some embodiments, a method comprises administering to a subject in need thereof a therapeutically effective amount of a neoantigenic polypeptide or polynucleotide described herein that delivers an exogenous polypeptide comprising at least one antigenic peptide to a tumor cell, wherein the neoepitope is presented on the surface of the tumor cell, and an immune response against the tumor cell is induced. In some embodiments, the immune response against the tumor cell is increased. In some embodiments, the neoantigenic polypeptide or polynucleotide delivers an exogenous polypeptide comprising at least one neoantigenic peptide to a tumor cell, wherein the neoepitope is presented on the surface of the tumor cell, and tumor growth is inhibited.

In some embodiments, a method comprises administering to a subject in need thereof a therapeutically effective amount of a neoantigenic polypeptide or polynucleotide described herein that delivers an exogenous polypeptide comprising at least one neoantigenic peptide to a tumor cell, wherein the neoepitope derived from the at least one neoantigenic peptide is presented on the surface of the tumor cell, and T cell killing directed against the tumor cell is induced. In some embodiments, T cell killing directed against the tumor cell is enhanced. In some embodiments, T cell killing directed against the tumor cell is increased.

The present disclosure also provides methods for inhibiting growth of a tumor using a polybody vaccine described herein. In certain embodiments, a method of inhibiting growth of a tumor comprises contacting a cell mixture with the polybody vaccine in vitro. For example, an immortalized cell line or a cancer cell line mixed with immune cells (e.g., T cells) is cultured in medium to which the polybody vaccine is added. In some embodiments, a polybody vaccine comprising neoantigens activates killing of the tumor cells.

In certain embodiments, the subject is a human. In certain embodiments, the subject has a tumor or the subject had a tumor which was at least partially removed.

In some embodiments, a method of inhibiting growth of a tumor comprises redirecting an existing immune response to a new target, comprising administering to a subject a therapeutically effective amount of a polybody therapeutic, wherein the existing immune response is against an antigenic peptide delivered to the subject.

In certain embodiments, the tumor comprises cancer stem cells. In certain embodiments, the frequency of cancer stem cells in the tumor is reduced by administration of the polybody. In some embodiments, a method of reducing the frequency of cancer stem cells in a tumor in a subject, comprising administering to the subject a therapeutically effective amount of a polybody therapeutic is provided.

In addition, in some aspects the present disclosure provides a method of reducing the tumorigenicity of a tumor in a subject, comprising administering to the subject a therapeutically effective amount of a neoantigen therapeutic described herein. In certain embodiments, the tumor comprises cancer stem cells. In some embodiments, the tumorigenicity of a tumor is reduced by reducing the frequency of cancer stem cells in the tumor. In some embodiments, the methods comprise using the neoantigen therapeutic described herein. In certain embodiments, the frequency of cancer stem cells in the tumor is reduced by administration of a neoantigen therapeutic described herein.

In some embodiments, the tumor is a solid tumor. In certain embodiments, the tumor is a tumor selected from the group consisting of: colorectal tumor, pancreatic tumor, lung tumor, ovarian tumor, liver tumor, breast tumor, kidney tumor, prostate tumor, neuroendocrine tumor, gastrointestinal tumor, melanoma, cervical tumor, bladder tumor, glioblastoma, and head and neck tumor. In certain embodiments, the tumor is a colorectal tumor. In certain embodiments, the tumor is an ovarian tumor. In some embodiments, the tumor is a breast tumor. In some embodiments, the tumor is a lung tumor. In certain embodiments, the tumor is a pancreatic tumor. In certain embodiments, the tumor is a melanoma tumor. In some embodiments, the tumor is a solid tumor.

The present disclosure further provides methods for treating cancer in a subject comprising administering to the subject a therapeutically effective amount of a neoantigen therapeutic described herein.

In some embodiments, a method of treating cancer comprises redirecting an existing immune response to a new target, the method comprising administering to a subject a therapeutically effective amount of neoantigen therapeutic, wherein the existing immune response is against an antigenic peptide delivered to the cancer cell by the neoantigenic peptide.

The present disclosure provides for methods of treating cancer comprising administering to a subject a therapeutically effective amount of a neoantigen therapeutic described herein (e.g., a subject in need of treatment). In certain embodiments, the subject is a human. In certain embodiments, the subject has a cancerous tumor. In certain embodiments, the subject has had a tumor at least partially removed.

Subjects can be, for example, mammal, humans, pregnant women, elderly adults, adults, adolescents, pre-adolescents, children, toddlers, infants, newborn, or neonates. A subject can be a patient. In some cases, a subject can be a human. In some cases, a subject can be a child (i.e. a young human being below the age of puberty). In some cases, a subject can be an infant. In some cases, the subject can be a formula-fed infant. In some cases, a subject can be an individual enrolled in a clinical study. In some cases, a subject can be a laboratory animal, for example, a mammal, or a rodent. In some cases, the subject can be a mouse. In some cases, the subject can be an obese or overweight subject.

In some embodiments, the subject has previously been treated with one or more different cancer treatment modalities. In some embodiments, the subject has previously been treated with one or more of radiotherapy, chemotherapy, or immunotherapy. In some embodiments, the subject has been treated with one, two, three, four, or five lines of prior therapy. In some embodiments, the prior therapy is a cytotoxic therapy.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, neuroendocrine cancer, bladder cancer, glioblastoma, and head and neck cancer. In certain embodiments, the cancer is pancreatic cancer. In certain embodiments, the cancer is ovarian cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is melanoma. In some embodiments, the cancer is a solid cancer. In some embodiments, the cancer comprises a solid tumor.

In some embodiments, the cancer is a hematologic cancer. In some embodiment, the cancer is selected from the group consisting of: acute myelogenous leukemia (AML), Hodgkin lymphoma, multiple myeloma, T cell acute lymphoblastic leukemia (T-ALL), chronic lymphocytic leukemia (CLL), hairy cell leukemia, chronic myelogenous leukemia (CML), non-Hodgkin lymphoma, diffuse large B-cell lymphoma (DLBCL), mantle cell lymphoma (MCL), and cutaneous T cell lymphoma (CTCL).

In some embodiments, the neoantigen therapeutic is administered as a combination therapy. Combination therapy with two or more therapeutic agents uses agents that work by different mechanisms of action, although this is not required. Combination therapy using agents with different mechanisms of action can result in additive or synergetic effects. Combination therapy can allow for a lower dose of each agent than is used in monotherapy, thereby reducing toxic side effects and/or increasing the therapeutic index of the agent(s). Combination therapy can decrease the likelihood that resistant cancer cells will develop. In some embodiments, combination therapy comprises a therapeutic agent that affects the immune response (e.g., enhances or activates the response) and a therapeutic agent that affects (e.g., inhibits or kills) the tumor/cancer cells.

In some instances, an immunogenic pharmaceutical composition can be administered with an additional agent. The choice of the additional agent can depend, at least in part, on the condition being treated. The additional agent can include, for example, a checkpoint inhibitor agent such as an anti-PD1, anti-CTLA4, anti-PD-L1, anti CD40, or anti-TIM3 agent (e.g., an anti-PD1, anti-CTLA4, anti-PD-L1, anti CD40, or anti-TIM3 antibody); or any agents having a therapeutic effect for a pathogen infection (e.g. viral infection), including, e.g., drugs used to treat inflammatory conditions such as an NSAID, e.g., ibuprofen, naproxen, acetaminophen, ketoprofen, or aspirin. For example, the checkpoint inhibitor can be a PD-1/PD-L1 antagonist selected from the group consisting of: nivolumab (ONO-4538/BMS-936558, MDX1 106, OPDIVO), pembrolizumab (MK-3475, KEYTRUDA), pidilizumab (CT-011), and MPDL3280A (ROCHE). As another example, formulations can additionally contain one or more supplements, such as vitamin C, E or other anti-oxidants.

The methods of the disclosure can be used to treat any type of cancer known in the art. Non-limiting examples of cancers to be treated by the methods of the present disclosure can include melanoma (e.g., metastatic malignant melanoma), renal cancer (e.g., clear cell carcinoma), prostate cancer (e.g., hormone refractory prostate adenocarcinoma), pancreatic adenocarcinoma, breast cancer, colon cancer, lung cancer (e.g., non-small cell lung cancer), esophageal cancer, squamous cell carcinoma of the head and neck, liver cancer, ovarian cancer, cervical cancer, thyroid cancer, glioblastoma, glioma, leukemia, lymphoma, and other neoplastic malignancies.

Additionally, the disease or condition provided herein includes refractory or recurrent malignancies whose growth may be inhibited using the methods of treatment of the present disclosure. In some embodiments, a cancer to be treated by the methods of treatment of the present disclosure is selected from the group consisting of carcinoma, squamous carcinoma, adenocarcinoma, sarcomata, endometrial cancer, breast cancer, ovarian cancer, cervical cancer, fallopian tube cancer, primary peritoneal cancer, colon cancer, colorectal cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, glioblastoma, glioma, squamous cell carcinoma of the head and neck, prostate cancer, pancreatic cancer, mesothelioma, sarcoma, hematological cancer, leukemia, lymphoma, neuroma, and combinations thereof. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma. In some embodiments, a cancer to be treated by the methods of the present disclosure is breast cancer. In some embodiments, a cancer to be treated by the methods of treatment of the present disclosure is triple negative breast cancer (TNBC). In some embodiments, a cancer to be treated by the methods of treatment of the present disclosure is ovarian cancer. In some embodiments, a cancer to be treated by the methods of treatment of the present disclosure is colorectal cancer.

In some embodiments, a patient or population of patients to be treated with a pharmaceutical composition of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, or Merkel cell carcinoma. In some embodiments, a patient or population of patients to be treated with a pharmaceutical composition of the present disclosure have a hematological cancer. In some embodiments, the patient has a hematological cancer such as Diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”). In some embodiments, a patient or population of patients to be treated having the cancer selected from the group consisting of ovarian cancer, lung cancer and melanoma.

Specific examples of cancers that can be prevented and/or treated in accordance with present disclosure include, but are not limited to, the following: renal cancer, kidney cancer, glioblastoma multiforme, metastatic breast cancer; breast carcinoma; breast sarcoma; neurofibroma; neurofibromatosis; pediatric tumors; neuroblastoma; malignant melanoma; carcinomas of the epidermis; leukemias such as but not limited to, acute leukemia, acute lymphocytic leukemia, acute myelocytic leukemias such as myeloblastic, promyelocytic, myelomonocytic, monocytic, erythroleukemia leukemias and myclodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenstrom's macroglobulinemia; monoclonal gammopathy of undetermined significance; benign monoclonal gammopathy; heavy chain disease; bone cancer and connective tissue sarcomas such as but not limited to bone sarcoma, myeloma bone disease, multiple myeloma, cholesteatoma-induced bone osteosarcoma, Paget's disease of bone, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangio sarcoma, neurilemmoma, rhabdomyosarcoma, and synovial sarcoma; brain tumors such as but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, and primary brain lymphoma; breast cancer including but not limited to adenocarcinoma, lobular (small cell) carcinoma, intraductal carcinoma, medullary breast cancer, mucinous breast cancer, tubular breast cancer, papillary breast cancer, Paget's disease (including juvenile Paget's disease) and inflammatory breast cancer; adrenal cancer such as but not limited to pheochromocytom and adrenocortical carcinoma; thyroid cancer such as but not limited to papillary or follicular thyroid cancer, medullary thyroid cancer and anaplastic thyroid cancer; pancreatic cancer such as but not limited to, insulinoma, gastrinoma, glucagonoma, vipoma, somatostatin-secreting tumor, and carcinoid or islet cell tumor; pituitary cancers such as but limited to Cushing's disease, prolactin-secreting tumor, acromegaly, and diabetes insipius; eye cancers such as but not limited to ocular melanoma such as iris melanoma, choroidal melanoma, and cilliary body melanoma, and retinoblastoma; vaginal cancers such as squamous cell carcinoma, adenocarcinoma, and melanoma; vulvar cancer such as squamous cell carcinoma, melanoma, adenocarcinoma, basal cell carcinoma, sarcoma, and Paget's disease; cervical cancers such as but not limited to, squamous cell carcinoma, and adenocarcinoma; uterine cancers such as but not limited to endometrial carcinoma and uterine sarcoma; ovarian cancers such as but not limited to, ovarian epithelial carcinoma, borderline tumor, germ cell tumor, and stromal tumor; cervical carcinoma; esophageal cancers such as but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers such as but not limited to, adenocarcinoma, fungating (polypoid), ulcerating, superficial spreading, diffusely spreading, malignant lymphoma, liposarcoma, fibrosarcoma, and carcinosarcoma; colon cancers; colorectal cancer, KRAS mutated colorectal cancer; colon carcinoma; rectal cancers; liver cancers such as but not limited to hepatocellular carcinoma and hepatoblastoma, gallbladder cancers such as adenocarcinoma; cholangiocarcinomas such as but not limited to pappillary, nodular, and diffuse; lung cancers such as KRAS-mutated non-small cell lung cancer, non-small cell lung cancer, squamous cell carcinoma (epidermoid carcinoma), adenocarcinoma, large-cell carcinoma and small-cell lung cancer; lung carcinoma; testicular cancers such as but not limited to germinal tumor, seminoma, anaplastic, classic (typical), spermatocytic, nonseminoma, embryonal carcinoma, teratoma carcinoma, choriocarcinoma (yolk-sac tumor), prostate cancers such as but not limited to, androgen-independent prostate cancer, androgen-dependent prostate cancer, adenocarcinoma, leiomyosarcoma, and rhabdomyosarcoma; penal cancers; oral cancers such as but not limited to squamous cell carcinoma; basal cancers; salivary gland cancers such as but not limited to adenocarcinoma, mucoepidermoid carcinoma, and adenoidcystic carcinoma; pharynx cancers such as but not limited to squamous cell cancer, and verrucous; skin cancers such as but not limited to, basal cell carcinoma, squamous cell carcinoma and melanoma, superficial spreading melanoma, nodular melanoma, lentigo malignant melanoma, acrallentiginous melanoma; kidney cancers such as but not limited to renal cell cancer, adenocarcinoma, hypernephroma, fibrosarcoma, transitional cell cancer (renal pelvis and/or uterer); renal carcinoma; Wilms' tumor; bladder cancers such as but not limited to transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

Cancers include, but are not limited to, B cell cancer, e.g., multiple myeloma, Waldenstrom's macroglobulinemia, the heavy chain diseases, such as, for example, alpha chain disease, gamma chain disease, and mu chain disease, benign monoclonal gammopathy, and immunocytic amyloidosis, melanomas, breast cancer, lung cancer, bronchus cancer, colorectal cancer, prostate cancer (e.g., metastatic, hormone refractory prostate cancer), pancreatic cancer, stomach cancer, ovarian cancer, urinary bladder cancer, brain or central nervous system cancer, peripheral nervous system cancer, esophageal cancer, cervical cancer, uterine or endometrial cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. Other non-limiting examples of types of cancers applicable to the methods encompassed by the present disclosure include human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, colorectal cancer, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, liver cancer, choriocarcinoma, seminoma, embryonal carcinoma, Wilms' tumor, cervical cancer, bone cancer, brain tumor, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia, and heavy chain disease. In some embodiments, the cancer whose phenotype is determined by the method of the present disclosure is an epithelial cancer such as, but not limited to, bladder cancer, breast cancer, cervical cancer, colon cancer, gynecologic cancers, renal cancer, laryngeal cancer, lung cancer, oral cancer, head and neck cancer, ovarian cancer, pancreatic cancer, prostate cancer, or skin cancer. In other embodiments, the cancer is breast cancer, prostate cancer, lung cancer, or colon cancer. In still other embodiments, the epithelial cancer is non-small-cell lung cancer, nonpapillary renal cell carcinoma, cervical carcinoma, ovarian carcinoma (e.g., serous ovarian carcinoma), or breast carcinoma. The epithelial cancers may be characterized in various other ways including, but not limited to, serous, endometrioid, mucinous, clear cell, brenner, or undifferentiated. In some embodiments, the present disclosure is used in the treatment, diagnosis, and/or prognosis of lymphoma or its subtypes, including, but not limited to, mantle cell lymphoma. Lymphoproliferative disorders are also considered to be proliferative diseases.

In some embodiments, the combination of an agent described herein and at least one additional therapeutic agent results in additive or synergistic results. In some embodiments, the combination therapy results in an increase in the therapeutic index of the agent. In some embodiments, the combination therapy results in an increase in the therapeutic index of the additional therapeutic agent(s). In some embodiments, the combination therapy results in a decrease in the toxicity and/or side effects of the agent. In some embodiments, the combination therapy results in a decrease in the toxicity and/or side effects of the additional therapeutic agent(s).

In certain embodiments, in addition to administering a neoantigen therapeutic described herein, the method or treatment further comprises administering at least one additional therapeutic agent. An additional therapeutic agent can be administered prior to, concurrently with, and/or subsequently to, administration of the agent. In some embodiments, the at least one additional therapeutic agent comprises 1, 2, 3, or more additional therapeutic agents.

Therapeutic agents that can be administered in combination with the neoantigen therapeutic described herein include chemotherapeutic agents. Thus, in some embodiments, the method or treatment involves the administration of an agent described herein in combination with a chemotherapeutic agent or in combination with a cocktail of chemotherapeutic agents. Treatment with an agent can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4th Edition, 2008, M. C. Perry, Editor, Lippincott, Williams & Wilkins, Philadelphia, PA.

Useful classes of chemotherapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor.

Chemotherapeutic agents useful in the present disclosure include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; ibandronate; CPT11; topoisomerase inhibitor RFS 2000; difluoromethylornithine (DMFO); retinoic acid; esperamicins; capecitabine (XELODA); and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON); and anti-androgens such as flutamide, nilutamide, bicalutamide, leuprolide, and goserelin; and pharmaceutically acceptable salts, acids or derivatives of any of the above. In certain embodiments, the additional therapeutic agent is cisplatin. In certain embodiments, the additional therapeutic agent is carboplatin.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In some embodiments, the additional therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6 mercaptopurine, azathioprine, 6-thioguanine, pentostatin, fludarabine phosphate, and cladribine, as well as pharmaceutically acceptable salts, acids, or derivatives of any of these. In certain embodiments, the additional therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, vinblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, the additional therapeutic agent is paclitaxel. In some embodiments, the additional therapeutic agent is albumin-bound paclitaxel.

In some embodiments, an additional therapeutic agent comprises an agent such as a small molecule. For example, treatment can involve the combined administration of an agent of the present disclosure with a small molecule that acts as an inhibitor against tumor-associated antigens including, but not limited to, EGFR, HER2 (ErbB2), and/or VEGF. In some embodiments, an agent of the present disclosure is administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor. In another embodiment, the additional therapeutic agent is chemotherapy or other inhibitors that reduce the number of Treg cells. In certain embodiments, the therapeutic agent is cyclophosphamide or an anti-CTLA4 antibody. In another embodiment, the additional therapeutic reduces the presence of myeloid-derived suppressor cells. In a further embodiment, the additional therapeutic is carbotaxol. In another embodiment, the additional therapeutic agent shifts cells to a T helper 1 response. In a further embodiment, the additional therapeutic agent is ibrutinib.

In some embodiments, an additional therapeutic agent comprises a biological molecule, such as an antibody. For example, treatment can involve the combined administration of an agent of the present disclosure with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

The agents and compositions provided herein may be used alone or in combination with conventional therapeutic regimens such as surgery, irradiation, chemotherapy and/or bone marrow transplantation (autologous, syngeneic, allogeneic or unrelated). A set of tumor antigens can be useful, e.g., in a large fraction of cancer patients.

In some embodiments, at least one or more chemotherapeutic agents may be administered in addition to the composition comprising an immunogenic vaccine. In some embodiments, the one or more chemotherapeutic agents may belong to different classes of chemotherapeutic agents.

Examples of chemotherapy agents include, but are not limited to, alkylating agents such as nitrogen mustards (e.g. mechlorethamine (nitrogen mustard), chlorambucil, cyclophosphamide (Cytoxan®), ifosfamide, and melphalan); nitrosoureas (e.g. N-Nitroso-N-methylurea, streptozocin, carmustine (BCNU), lomustine, and semustine); alkyl sulfonates (e.g. busulfan); tetrazines (e.g. dacarbazine (DTIC), mitozolomide and temozolomide (Temodar®)); aziridines (e.g. thiotepa, mytomycin and diaziquone); and platinum drugs (e.g. cisplatin, carboplatin, and oxaliplatin); non-classical alkylating agents such as procarbazine and altretamine (hexamethylmelamine); anti-metabolite agents such as 5-fluorouracil (5-FU), 6-mercaptopurine (6-MP), capecitabine (Xeloda®), cladribine, clofarabine, cytarabine (Ara-C®), decitabine, floxuridine, fludarabine, nelarabine, gemcitabine (Gemzar®), hydroxyurea, methotrexate, pemetrexed (Alimta®), pentostatin, thioguanine, Vidaza; anti-microtubule agents such as vinca alkaloids (e.g. vincristine, vinblastine, vinorelbine, vindesine and vinflunine); taxanes (e.g. paclitaxel (Taxol®), docetaxel (Taxotere®)); podophyllotoxin (e.g. etoposide and teniposide); epothilones (e.g. ixabepilone (Ixempra®)); estramustine (Emcyt®); anti-tumor antibiotics such as anthracyclines (e.g. daunorubicin, doxorubicin (Adriamycin®, epirubicin, idarubicin); actinomycin-D; and bleomycin; topoisomerase I inhibitors such as topotecan and irinotecan (CPT-11); topoisomerase II inhibitors such as etoposide (VP-16), teniposide, mitoxantrone, novobiocin, merbarone and aclarubicin; corticosteroids such as prednisone, methylprednisolone (Solumedrol®), and dexamethasone (Decadron®); L-asparaginase; bortezomib (Velcade®); immunotherapeutic agents such as rituximab (Rituxan®), alemtuzumab (Campath®), thalidomide, lenalidomide (Revlimid®), BCG, interleukin-2, interferon-alfa and cancer vaccines such as Provenge®; hormone therapeutic agents such as fulvestrant (Faslodex®), tamoxifen, toremifene (Fareston®), anastrozole (Arimidex®), exemestan (Aromasin®), letrozole (Femara®), megestrol acetate (Megace®), estrogens, bicalutamide (Casodex®), flutamide (Eulexin®), nilutamide (Nilandron®), leuprolide (Lupron®) and goserelin (Zoladex®); differentiating agents such as retinoids, tretinoin (ATRA or Atralin®), bexarotene (Targretin®) and arsenic trioxide (Arsenox®); and targeted therapeutic agents such as imatinib (Gleevec®), gefitinib (Iressa®) and sunitinib (Sutent®). In some embodiments, the chemotherapy is a cocktail therapy. Examples of a cocktail therapy includes, but is not limited to, CHOP/R-CHOP (rituxan, cyclophosphamide, hydroxydoxorubicin, vincristine, and prednisone), EPOCH (etoposide, prednisone, vincristine, cyclophosphamide, hydroxydoxorubicin), Hyper-CVAD (cyclophosphamide, vincristine, hydroxydoxorubicin, dexamethasone), FOLFOX (fluorouracil (5-FU), leucovorin, oxaliplatin), ICE (ifosfamide, carboplatin, etoposide), DHAP (high-dose cytarabine [ara-C], dexamethasone, cisplatin), ESHAP (etoposide, methylprednisolone, cytarabine [ara-C], cisplatin) and CMF (cyclophosphamide, methotrexate, fluouracil).

In certain embodiments, an additional therapeutic agent comprises a second immunotherapeutic agent. In some embodiments, the additional immunotherapeutic agent includes, but is not limited to, a colony stimulating factor, an interleukin, an antibody that blocks immunosuppressive functions (e.g., an anti-CTLA-4 antibody, anti-CD28 antibody, anti-CD3 antibody, anti-PD-1 antibody, anti-PD-L1 antibody, anti-TIGIT antibody), an antibody that enhances immune cell functions (e.g., an anti-GITR antibody, an anti-OX-40 antibody, an anti-CD40 antibody, or an anti-4-1BB antibody), a toll-like receptor (e.g., TLR3 TLR7, TLR9), a soluble ligand (e.g., GITRL, GITRL-Fc, OX-40L, OX-40L-Fc, CD40L, CD40L-Fc, 4-1BB ligand, or 4-1BB ligand-Fc), or a member of the B7 family (e.g., CD80, CD86). In some embodiments, the additional immunotherapeutic agent targets CTLA-4, CD28, CD3, PD-1, PD-L1, TIGIT, GITR, OX-40, CD-40, or 4-1BB.

In some embodiments, the additional therapeutic agent is an immune checkpoint inhibitor. In some embodiments, the immune checkpoint inhibitor is an anti-PD-1 antibody, an anti-PD-L1 antibody, an anti-CTLA-4 antibody, an anti-CD28 antibody, an anti-TIGIT antibody, an anti-LAG3 antibody, an anti-TIM3 antibody, an anti-GITR antibody, an anti-4-1BB antibody, or an anti-OX-40 antibody. In some embodiments, the additional therapeutic agent is an anti-TIGIT antibody. In some embodiments, the additional therapeutic agent is an anti-PD-1 antibody selected from the group consisting of: nivolumab (OPDIVO), pembrolizumab (KEYTRUDA), pidilzumab, MEDI0680, REGN2810, BGB-A317, and PDR001. In some embodiments, the additional therapeutic agent is an anti-PD-L1 antibody selected from the group consisting of: BMS935559 (MDX-1105), atexolizumab (MPDL3280A), durvalumab (MEDI4736), and avelumab (MSB0010718C). In some embodiments, the additional therapeutic agent is an anti-CTLA-4 antibody selected from the group consisting of: ipilimumab (YERVOY) and tremelimumab. In some embodiments, the additional therapeutic agent is an anti-LAG-3 antibody selected from the group consisting of: BMS-986016 and LAG525. In some embodiments, the additional therapeutic agent is an anti-OX-40 antibody selected from the group consisting of: MEDI6469, MEDI0562, and MOXR0916. In some embodiments, the additional therapeutic agent is an anti-4-1BB antibody selected from the group consisting of: PF-05082566.

In some embodiments, the neoantigen therapeutic can be administered in combination with a biologic molecule selected from the group consisting of: FLT3 ligand, adrenomedullin (AM), angiopoietin (Ang), BMPs, BDNF, EGF, erythropoietin (EPO), FGF, GDNF, granulocyte colony stimulating factor (G-CSF), granulocyte-macrophage colony stimulating factor (GM-CSF), macrophage colony stimulating factor (M-CSF), stem cell factor (SCF), GDF9, HGF, HDGF, IGF, migration-stimulating factor, myostatin (GDF-8), NGF, neurotrophins, PDGF, thrombopoietin, TGF-α, TGF-β, TNF-α, VEGF, PIGF, gamma-IFN, IL-1, IL-2, IL-3, IL-4, IL-5, IL-6, IL-7, IL-12, IL-15, IL-18, or an agent that reduces myeloid-derived suppressor cells (MDSCs) or immunosuppressive macrophoage populations.

In some embodiments, treatment with a neoantigen therapeutic described herein can be accompanied by surgical removal of tumors, removal of cancer cells, or any other surgical therapy deemed necessary by a treating physician.

In certain embodiments, treatment involves the administration of a neoantigen therapeutic described herein in combination with radiation therapy. Treatment with an agent can occur prior to, concurrently with, or subsequent to administration of radiation therapy. Dosing schedules for such radiation therapy can be determined by the skilled medical practitioner.

Combined administration can include co-administration, either in a single pharmaceutical formulation or using separate formulations, or consecutive administration in either order but generally within a time period such that all active agents can exert their biological activities simultaneously or sequentially, dependent on the agent.

It will be appreciated that the combination of a neoantigen therapeutic described herein and at least one additional therapeutic agent can be administered in any order or concurrently. In some embodiments, the agent will be administered to patients that have previously undergone treatment with a second therapeutic agent. In certain other embodiments, the neoantigen therapeutic and a second therapeutic agent will be administered substantially simultaneously or concurrently. For example, a subject can be given an agent while undergoing a course of treatment with a second therapeutic agent (e.g., chemotherapy). In certain embodiments, a neoantigen therapeutic will be administered within 1 year of the treatment with a second therapeutic agent. It will further be appreciated that the two (or more) agents or treatments can be administered to the subject within a matter of hours or minutes (i.e., substantially simultaneously).

For the treatment of a disease, the appropriate dosage of a neoantigen therapeutic described herein depends on the type of disease to be treated, the severity and course of the disease, the responsiveness of the disease, whether the agent is administered for therapeutic or preventative purposes, previous therapy, the patient's clinical history, and so on, all at the discretion of the treating physician. The neoantigen therapeutic can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual agent. The administering physician can determine optimum dosages, dosing methodologies, and repetition rates.

In some embodiments, a neoantigen therapeutic can be administered at an initial higher “loading” dose, followed by one or more lower doses. In some embodiments, the frequency of administration can also change. In some embodiments, a dosing regimen can comprise administering an initial dose, followed by additional doses (or “maintenance” doses) once a week, once every two weeks, once every three weeks, or once every month. For example, a dosing regimen can comprise administering an initial loading dose, followed by a weekly maintenance dose of, for example, one-half of the initial dose. Or a dosing regimen can comprise administering an initial loading dose, followed by maintenance doses of, for example one-half of the initial dose every other week. Or a dosing regimen can comprise administering three initial doses for 3 weeks, followed by maintenance doses of, for example, the same amount every other week.

As is known to those of skill in the art, administration of any therapeutic agent can lead to side effects and/or toxicities. In some cases, the side effects and/or toxicities are so severe as to preclude administration of the particular agent at a therapeutically effective dose. In some cases, therapy must be discontinued, and other agents can be tried. However, many agents in the same therapeutic class display similar side effects and/or toxicities, meaning that the patient either has to stop therapy, or if possible, suffer from the unpleasant side effects associated with the therapeutic agent.

In some embodiments, the dosing schedule can be limited to a specific number of administrations or “cycles”. In some embodiments, the agent is administered for 3, 4, 5, 6, 7, 8, or more cycles. For example, the agent is administered every 2 weeks for 6 cycles, the agent is administered every 3 weeks for 6 cycles, the agent is administered every 2 weeks for 4 cycles, the agent is administered every 3 weeks for 4 cycles, etc. Dosing schedules can be decided upon and subsequently modified by those skilled in the art.

The present disclosure provides methods of administering to a subject a neoantigen therapeutic described herein comprising using an intermittent dosing strategy for administering one or more agents, which can reduce side effects and/or toxicities associated with administration of an agent, chemotherapeutic agent, etc. In some embodiments, a method for treating cancer in a human subject comprises administering to the subject a therapeutically effective dose of a neoantigen therapeutic in combination with a therapeutically effective dose of a chemotherapeutic agent, wherein one or both of the agents are administered according to an intermittent dosing strategy. In some embodiments, a method for treating cancer in a human subject comprises administering to the subject a therapeutically effective dose of a neoantigen therapeutic in combination with a therapeutically effective dose of a second immunotherapeutic agent, wherein one or both of the agents are administered according to an intermittent dosing strategy. In some embodiments, the intermittent dosing strategy comprises administering an initial dose of a neoantigen therapeutic to the subject, and administering subsequent doses of the agent about once every 2 weeks. In some embodiments, the intermittent dosing strategy comprises administering an initial dose of a neoantigen therapeutic to the subject, and administering subsequent doses of the agent about once every 3 weeks. In some embodiments, the intermittent dosing strategy comprises administering an initial dose of a neoantigen therapeutic to the subject, and administering subsequent doses of the agent about once every 4 weeks. In some embodiments, the agent is administered using an intermittent dosing strategy and the additional therapeutic agent is administered weekly.

The present disclosure provides compositions comprising the neoantigen therapeutic described herein. The present disclosure also provides pharmaceutical compositions comprising a neoantigen therapeutic described herein and a pharmaceutically acceptable vehicle. In some embodiments, the pharmaceutical compositions find use in immunotherapy. In some embodiments, the compositions find use in inhibiting tumor growth. In some embodiments, the pharmaceutical compositions find use in inhibiting tumor growth in a subject (e.g., a human patient). In some embodiments, the compositions find use in treating cancer. In some embodiments, the pharmaceutical compositions find use in treating cancer in a subject (e.g., a human patient).

Formulations are prepared for storage and use by combining a neoantigen therapeutic of the present disclosure with a pharmaceutically acceptable vehicle (e.g., a carrier or excipient). Those of skill in the art generally consider pharmaceutically acceptable carriers, excipients, and/or stabilizers to be inactive ingredients of a formulation or pharmaceutical composition. Exemplary formulations are listed in WO 2015/095811.

Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 22st Edition, 2012, Pharmaceutical Press, London.). In some embodiments, the vehicle is 5% dextrose in water.

The pharmaceutical compositions described herein can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intra-arterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories.

The neoantigenic peptides described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22st Edition, 2012, Pharmaceutical Press, London.

In certain embodiments, pharmaceutical formulations include a neoantigen therapeutic described herein complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations comprising the neoantigenic peptides described herein can be produced. Suitable examples of sustained-release preparations include semi-permeable matrices of solid hydrophobic polymers containing an agent, where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The present disclosure provides methods of treatment comprising an immunogenic vaccine. Methods of treatment for a disease (such as cancer or a viral infection) are provided. A method can comprise administering to a subject an effective amount of a composition comprising an immunogenic antigen. In some embodiments, the antigen comprises a viral antigen. In some embodiments, the antigen comprises a tumor antigen.

Non-limiting examples of vaccines that can be prepared include a peptide-based vaccine, a nucleic acid-based vaccine, an antibody based vaccine, a T cell based vaccine, and an antigen-presenting cell based vaccine.

Vaccine compositions can be formulated using one or more physiologically acceptable carriers including excipients and auxiliaries which facilitate processing of the active agents into preparations which can be used pharmaceutically. Proper formulation can be dependent upon the route of administration chosen. Any of the well-known techniques, carriers, and excipients can be used as suitable and as understood in the art.

In some cases, the vaccine composition is formulated as a peptide-based vaccine, a protein based vaccine, a nucleic acid-based vaccine, an antibody based vaccine, or a cell based vaccine. For example, a vaccine composition can include naked cDNA in cationic lipid formulations; lipopeptides (e.g., Vitiello, A. et al., J. Clin. Invest. 95:341, 1995), naked cDNA or peptides, encapsulated e.g., in poly(DL-lactide-co-glycolide) (“PLG”) microspheres (see, e.g., Eldridge, et al., Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tarn, J. P., J. Immunol. Methods 196:17-32, 1996). Sometimes, a vaccine is formulated as a peptide-based vaccine, or nucleic acid based vaccine in which the nucleic acid encodes the polypeptides. Sometimes, a vaccine is formulated as an antibody based vaccine. Sometimes, a vaccine is formulated as a cell based vaccine.

The amino acid sequence of an identified disease-specific immunogenic neoantigen peptide can be used to develop a pharmaceutically acceptable composition. The source of antigen can be, but is not limited to, natural or synthetic proteins, including glycoproteins, peptides, and superantigens; antibody/antigen complexes; lipoproteins; RNA or a translation product thereof; and DNA or a polypeptide encoded by the DNA. The source of antigen may also comprise non-transformed, transformed, transfected, or transduced cells or cell lines. Cells may be transformed, transfected, or transduced using any of a variety of expression or retroviral vectors known to those of ordinary skill in the art that may be employed to express recombinant antigens. Expression may also be achieved in any appropriate host cell that has been transformed, transfected, or transduced with an expression or retroviral vector containing a DNA molecule encoding recombinant antigen(s). Any number of transfection, transformation, and transduction protocols known to those in the art may be used. Recombinant vaccinia vectors and cells infected with the vaccinia vector, may be used as a source of antigen.

A composition can comprise a synthetic disease-specific immunogenic neoantigen peptide. A composition can comprise two or more disease-specific immunogenic neoantigen peptides. A composition may comprise a precursor to a disease-specific immunogenic peptide (such as a protein, peptide, DNA and RNA). A precursor to a disease-specific immunogenic peptide can generate or be generated to the identified disease-specific immunogenic neoantigen peptide. In some embodiments, a therapeutic composition comprises a precursor of an immunogenic peptide. The precursor to a disease-specific immunogenic peptide can be a pro-drug. In some embodiments, the composition comprising a disease-specific immunogenic neoantigen peptide may further comprise an adjuvant. For example, the neoantigen peptide can be utilized as a vaccine. In some embodiments, an immunogenic vaccine may comprise a pharmaceutically acceptable immunogenic neoantigen peptide. In some embodiments, an immunogenic vaccine may comprise a pharmaceutically acceptable precursor to an immunogenic neoantigen peptide (such as a protein, peptide, DNA and RNA). In some embodiments, a method of treatment comprises administering to a subject an effective amount of an antibody specifically recognizing an immunogenic neoantigen peptide. In some embodiments, a method of treatment comprises administering to a subject an effective amount of a soluble TCR or TCR analog specifically recognizing an immunogenic neoantigen peptide.

The methods described herein are particularly useful in the personalized medicine context, where immunogenic neoantigen peptides are used to develop therapeutics (such as vaccines or therapeutic antibodies) for the same individual. Thus, a method of treating a disease in a subject can comprise identifying an immunogenic neoantigen peptide in a subject according to the methods described herein; and synthesizing the peptide (or a precursor thereof); and administering the peptide or an antibody specifically recognizing the peptide to the subject. In some embodiments, an expression pattern of an immunogenic neoantigen can serve as the essential basis for the generation of patient specific vaccines. In some embodiments, an expression pattern of an immunogenic neoantigen can serve as the essential basis for the generation of a vaccine for a group of patients with a particular disease. Thus, particular diseases, e.g., particular types of tumors, can be selectively treated in a patient group.

In some embodiments, the peptides described herein are structurally normal antigens that can be recognized by autologous anti-disease T cells in a large patient group. In some embodiments, an antigen-expression pattern of a group of diseased subjects whose disease expresses structurally normal neoantigens is determined.

In some embodiments, the peptides described herein comprises a first peptide comprising a first neoepitope of a protein and a second peptide comprising a second neoepitope of the same protein, wherein the first peptide is different from the second peptide, and wherein the first neoepitope comprises a mutation and the second neoepitope comprises the same mutation. In some embodiments, the peptides described herein comprises a first peptide comprising a first neoepitope of a first region of a protein and a second peptide comprising a second neoepitope of a second region of the same protein, wherein the first region comprises at least one amino acid of the second region, wherein the first peptide is different from the second peptide and wherein the first neoepitope comprises a first mutation and the second neoepitope comprises a second mutation. In some embodiments, the first mutation and the second mutation are the same. In some embodiments, the mutation is selected from the group consisting of a point mutation, a splice-site mutation, a frameshift mutation, a read-through mutation, a gene fusion mutation and any combination thereof.

There are a variety of ways in which to produce immunogenic neoantigens. Proteins or peptides may be made by any technique known to those of skill in the art, including the expression of proteins, polypeptides or peptides through standard molecular biological techniques, the isolation of proteins or peptides from natural sources, in vitro translation, or the chemical synthesis of proteins or peptides. In general, such disease specific neoantigens may be produced either in vitro or in vivo. Immunogenic neoantigens may be produced in vitro as peptides or polypeptides, which may then be formulated into a personalized vaccine or immunogenic composition and administered to a subject. In vitro production of immunogenic neoantigens can comprise peptide synthesis or expression of a peptide/polypeptide from a DNA or RNA molecule in any of a variety of bacterial, eukaryotic, or viral recombinant expression systems, followed by purification of the expressed peptide/polypeptide. Alternatively, immunogenic neoantigens can be produced in vivo by introducing molecules (e.g., DNA, RNA, and viral expression systems) that encode an immunogenic neoantigen into a subject, whereupon the encoded immunogenic neoantigens are expressed. In some embodiments, a polynucleotide encoding an immunogenic neoantigen peptide can be used to produce the neoantigen peptide in vitro.

In some embodiments, a polynucleotide comprises a sequence with at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to a polynucleotide encoding an immunogenic neoantigen.

The polynucleotide may be, e.g., DNA, cDNA, PNA, CNA, RNA, single- and/or double-stranded, native or stabilized forms of polynucleotides, or combinations thereof. A nucleic acid encoding an immunogenic neoantigen peptide may or may not contain introns so long as it codes for the peptide. In some embodiments in vitro translation is used to produce the peptide.

Expression vectors comprising sequences encoding the neoantigen, as well as host cells containing the expression vectors, are also contemplated. Expression vectors suitable for use in the present disclosure can comprise at least one expression control element operationally linked to the nucleic acid sequence. The expression control elements are inserted in the vector to control and regulate the expression of the nucleic acid sequence. Examples of expression control elements are well known in the art and include, for example, the lac system, operator and promoter regions of phage lambda, yeast promoters and promoters derived from polyoma, adenovirus, retrovirus or SV40. Additional operational elements include, but are not limited to, leader sequences, termination codons, polyadenylation signals and any other sequences necessary or preferred for the appropriate transcription and subsequent translation of the nucleic acid sequence in the host system. It will be understood by one skilled in the art the correct combination of expression control elements will depend on the host system chosen. It will further be understood that the expression vector should contain additional elements necessary for the transfer and subsequent replication of the expression vector containing the nucleic acid sequence in the host system. Examples of such elements include, but are not limited to, origins of replication and selectable markers.

The neoantigen peptides may be provided in the form of RNA or cDNA molecules encoding the desired neoantigen peptides. One or more neoantigen peptides of the present disclosure may be encoded by a single expression vector. Generally, the DNA is inserted into an expression vector, such as a plasmid, in proper orientation and correct reading frame for expression, if necessary, the DNA may be linked to the appropriate transcriptional and translational regulatory control nucleotide sequences recognized by the desired host (e.g., bacteria), although such controls are generally available in the expression vector. The vector is then introduced into the host bacteria for cloning using standard techniques. Useful expression vectors for eukaryotic hosts, especially mammals or humans include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR 1, pBR322, pMB9 and their derivatives, wider host range plasmids, such as M13 and filamentous single-stranded DNA phages.

In embodiments, a DNA sequence encoding a polypeptide of interest can be constructed by chemical synthesis using an oligonucleotide synthesizer. Such oligonucleotides can be designed based on the amino acid sequence of the desired polypeptide and selecting those codons that are favored in the host cell in which the recombinant polypeptide of interest is produced. Standard methods can be applied to synthesize an isolated polynucleotide sequence encoding an isolated polypeptide of interest.

Suitable host cells for expression of a polypeptide include prokaryotes, yeast, insect or higher eukaryotic cells under the control of appropriate promoters. Prokaryotes include gram negative or gram positive organisms, for example E. coli or bacilli. Higher eukaryotic cells include established cell lines of mammalian origin. Cell-free translation systems can also be employed. Appropriate cloning and expression vectors for use with bacterial, fungal, yeast, and mammalian cellular hosts are well known in the art. Various mammalian or insect cell culture systems can be employed to express recombinant protein. Exemplary mammalian host cell lines include, but are not limited to COS-7, L cells, C127, 3T3, Chinese hamster ovary (CHO), 293, HeLa and BHK cell lines. Mammalian expression vectors can comprise nontranscribed elements such as an origin of replication, a suitable promoter and enhancer linked to the gene to be expressed, and other 5′ or 3′ flanking nontranscribed sequences, and 5′ or 3′ nontranslated sequences, such as necessary ribosome binding sites, a polyadenylation site, splice donor and acceptor sites, and transcriptional termination sequences.

The proteins produced by a transformed host can be purified according to any suitable method. Such standard methods include chromatography (e.g., ion exchange, affinity and sizing column chromatography, and the like), centrifugation, differential solubility, or by any other standard technique for protein purification. Affinity tags such as hexahistidine, maltose binding domain, influenza coat sequence, glutathione-S-transferase, and the like can be attached to the protein to allow easy purification by passage over an appropriate affinity column. Isolated proteins can also be physically characterized using such techniques as proteolysis, nuclear magnetic resonance and x-ray crystallography. Additionally, a specific antibody or fragment thereof, or other binding domain, capable of specifically binding to the immunogen or fragment thereof may used.

A vaccine can comprise an entity that binds a polypeptide sequence described herein. The entity can be an antibody. Antibody-based vaccine can be formulated using any of the well-known techniques, carriers, and excipients as suitable and as understood in the art. In some embodiments, the peptides described herein can be used for making neoantigen specific therapeutics such as antibody therapeutics. For example, neoantigens can be used to raise and/or identify antibodies specifically recognizing the neoantigens. These antibodies can be used as therapeutics. The antibody can be a natural antibody, a chimeric antibody, a humanized antibody, or can be an antibody fragment. The antibody may recognize one or more of the polypeptides described herein. In some embodiments, the antibody can recognize a polypeptide that has a sequence with at most 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity to a polypeptide described herein. In some embodiments, the antibody can recognize a polypeptide that has a sequence with at least 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to a polypeptide described herein. In some embodiments, the antibody can recognize a polypeptide sequence that is at least 30%, 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a length of a polypeptide described herein. In some embodiments, the antibody can recognize a polypeptide sequence that is at most 30%, 40%, 50%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% of a length of a polypeptide described herein.

The present disclosure also contemplates the use of nucleic acid molecules as vehicles for delivering neoantigen peptides/polypeptides to the subject in need thereof, in vivo, in the form of, e.g., DNA/RNA vaccines.

In some embodiments, the vaccine is a nucleic acid vaccine. In some embodiments, neoantigens can be administered to a subject by use of a plasmid. Plasmids may be introduced into animal tissues by a number of different methods, e.g., injection or aerosol instillation of naked DNA on mucosal surfaces, such as the nasal and lung mucosa. In some embodiments, physical delivery, such as with a “gene-gun” may be used. The exact choice of expression vectors can depend upon the peptide/polypeptides to be expressed, and is well within the skill of the ordinary artisan.

In some embodiments, the nucleic acid encodes an immunogenic peptide or peptide precursor. In some embodiments, the nucleic acid vaccine comprises sequences flanking the sequence coding the immunogenic peptide or peptide precursor. In some embodiments, the nucleic acid vaccine comprises more than one immunogenic epitope. In some embodiments, the nucleic acid vaccine is a DNA-based vaccine. In some embodiments, the nucleic acid vaccine is a RNA-based vaccine. In some embodiments, the RNA-based vaccine comprises mRNA. In some embodiments, the RNA-based vaccine comprises naked mRNA. In some embodiments, the RNA-based vaccine comprises modified mRNA (e.g., mRNA protected from degradation using protamine. mRNA containing modified 5′ CAP structure or mRNA containing modified nucleotides). In some embodiments, the RNA-based vaccine comprises single-stranded mRNA.

The polynucleotide may be substantially pure, or contained in a suitable vector or delivery system. Suitable vectors and delivery systems include viral, such as systems based on adenovirus, vaccinia virus, retroviruses, herpes virus, adeno-associated virus or hybrids containing elements of more than one virus. Non-viral delivery systems include cationic lipids and cationic polymers (e.g., cationic liposomes).

One or more neoantigen peptides can be encoded and expressed in vivo using a viral based system. Viral vectors may be used as recombinant vectors in the present disclosure, wherein a portion of the viral genome is deleted to introduce new genes without destroying infectivity of the virus. The viral vector of the present disclosure is a nonpathogenic virus. In some embodiments the viral vector has a tropism for a specific cell type in the mammal. In another embodiment, the viral vector of the present disclosure is able to infect professional antigen presenting cells such as dendritic cells and macrophages. In yet another embodiment of the present disclosure, the viral vector is able to infect any cell in the mammal. The viral vector may also infect tumor cells. Viral vectors used in the present disclosure include but is not limited to Poxvirus such as vaccinia virus, avipox virus, fowlpox virus and a highly attenuated vaccinia virus (Ankara or MVA), retrovirus, adenovirus, baculovirus and the like.

A vaccine can be delivered via a variety of routes. Delivery routes can include oral (including buccal and sub-lingual), rectal, nasal, topical, transdermal patch, pulmonary, vaginal, suppository, or parenteral (including intramuscular, intra-arterial, intrathecal, intradermal, intraperitoneal, subcutaneous and intravenous) administration or in a form suitable for administration by aerosolization, inhalation or insufflation. General information on drug delivery systems can be found in Ansel et al., Pharmaceutical Dosage Forms and Drug Delivery Systems (Lippencott Williams & Wilkins, Baltimore Md. (1999). The vaccine described herein can be administered to muscle, or can be administered via intradermal or subcutaneous injections, or transdermally, such as by iontophoresis. Epidermal administration of the vaccine can be employed.

In some instances, the vaccine can also be formulated for administration via the nasal passages. Formulations suitable for nasal administration, wherein the carrier is a solid, can include a coarse powder having a particle size, for example, in the range of about 10 to about 500 microns which is administered in the manner in which snuff is taken, i.e., by rapid inhalation through the nasal passage from a container of the powder held close up to the nose. The formulation can be a nasal spray, nasal drops, or by aerosol administration by nebulizer. The formulation can include aqueous or oily solutions of the vaccine.

The vaccine can be a liquid preparation such as a suspension, syrup or elixir. The vaccine can also be a preparation for parenteral, subcutaneous, intradermal, intramuscular or intravenous administration (e.g., injectable administration), such as a sterile suspension or emulsion.

The vaccine can include material for a single immunization, or may include material for multiple immunizations (i.e. a ‘multidose’ kit). The inclusion of a preservative is preferred in multidose arrangements. As an alternative (or in addition) to including a preservative in multidose compositions, the compositions can be contained in a container having an aseptic adaptor for removal of material.

The vaccine can be administered in a dosage volume of about 0.5 mL, although a half dose (i.e. about 0.25 mL) can be administered to children. Sometimes the vaccine can be administered in a higher dose e.g. about 1 ml.

The vaccine can be administered as a 1, 2, 3, 4, 5, 6, 7, 8, 9, 10, or more dose-course regimen. Sometimes, the vaccine is administered as a 1, 2, 3, or 4 dose-course regimen. Sometimes the vaccine is administered as a 1 dose-course regimen. Sometimes the vaccine is administered as a 2 dose-course regimen.

The administration of the first dose and second dose can be separated by about 0 day, 1 day, 2 days, 5 days, 7 days, 14 days, 21 days, 30 days, 2 months, 4 months, 6 months, 9 months, 1 year, 1.5 years, 2 years, 3 years, 4 years, or more.

The vaccine described herein can be administered every 1, 2, 3, 4, 5, 6, 7, 8, 9, 10, or more years. Sometimes, the vaccine described herein is administered every 2, 3, 4, 5, 6, 7, or more years. Sometimes, the vaccine described herein is administered every 4, 5, 6, 7, or more years. Sometimes, the vaccine described herein is administered once.

The dosage examples are not limiting and are only used to exemplify particular dosing regiments for administering a vaccine described herein. The effective amount for use in humans can be determined from animal models. For example, a dose for humans can be formulated to achieve circulating, liver, topical and/or gastrointestinal concentrations that have been found to be effective in animals. Based on animal data, and other types of similar data, those skilled in the art can determine the effective amounts of a vaccine composition appropriate for humans.

The effective amount when referring to an agent or combination of agents will generally mean the dose ranges, modes of administration, formulations, etc., that have been recommended or approved by any of the various regulatory or advisory organizations in the medical or pharmaceutical arts (e.g., FDA, AMA) or by the manufacturer or supplier.

In some aspects, the vaccine and kit described herein can be stored at between 2° C. and 8° C. In some instances, the vaccine is not stored frozen. In some instances, the vaccine is stored in temperatures of such as at −20° C. or −80° C. In some instances, the vaccine is stored away from sunlight.

### Kits

The neoantigen therapeutic described herein can be provided in kit form together with instructions for administration. Typically, the kit would include the desired neoantigen therapeutic in a container, in unit dosage form and instructions for administration. Additional therapeutics, for example, cytokines, lymphokines, checkpoint inhibitors, antibodies, can also be included in the kit. Other kit components that can also be desirable include, for example, a sterile syringe, booster dosages, and other desired excipients.

Kits and articles of manufacture are also provided herein for use with one or more methods described herein. The kits can contain one or more neoantigenic polypeptides comprising one or more neoepitopes. The kits can also contain nucleic acids that encode one or more of the peptides or proteins described herein, antibodies that recognize one or more of the peptides described herein, or APC-based cells activated with one or more of the peptides described herein. The kits can further contain adjuvants, reagents, and buffers necessary for the makeup and delivery of the vaccines.

The kits can also include a carrier, package, or container that is compartmentalized to receive one or more containers such as vials, tubes, and the like, each of the container(s) comprising one of the separate elements, such as the peptides and adjuvants, to be used in a method described herein. Suitable containers include, for example, bottles, vials, syringes, and test tubes. The containers can be formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and intended mode of administration and treatment. A kit typically includes labels listing contents and/or instructions for use, and package inserts with instructions for use. A set of instructions will also typically be included.

The present disclosure will be described in greater detail by way of specific examples. The following examples are offered for illustrative purposes, and are not intended to limit the present disclosure in any manner. Those of skill in the art will readily recognize a variety of non-critical parameters that can be changed or modified to yield alternative embodiments according to the present disclosure. All patents, patent applications, and printed publications listed herein are incorporated herein by reference in their entirety.

### EXAMPLES

These examples are provided for illustrative purposes only and not to limit the scope of the claims provided herein.

### Example 1—Polybody Design

### 1.1. Selection Criteria for the Scaffolding Domain

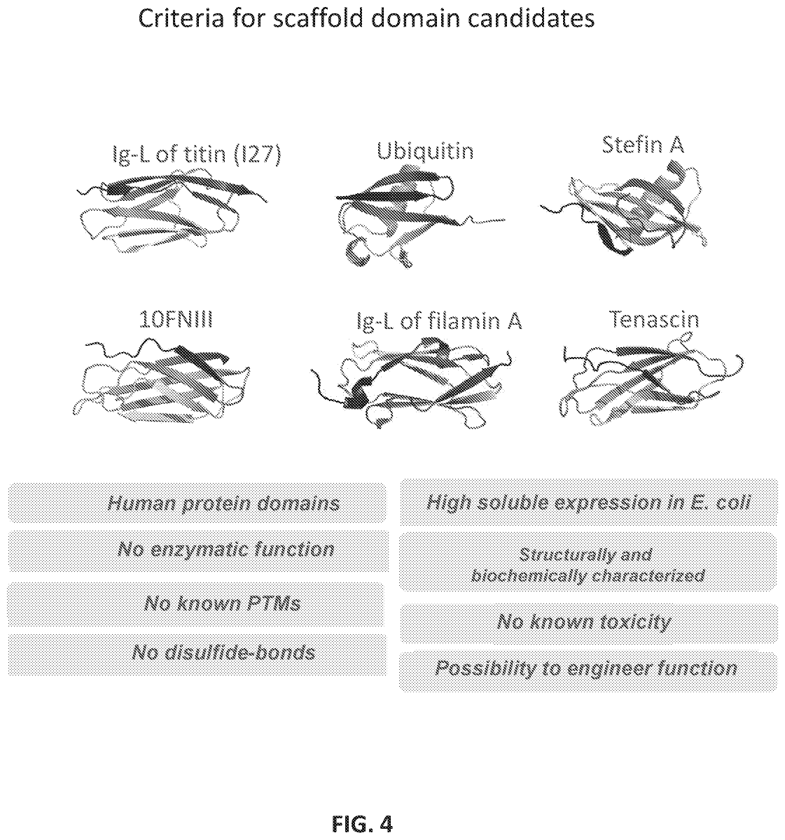

To identify suitable scaffolding domains to design a multidomain polybody, in some cases, several selection criteria can be considered (FIG. 4). In some embodiments, it is desirable to minimize the risk of unwanted immune responses, and in these situations, endogenous human proteins/protein domains or foreign proteins that are known to be non-immunogenic are preferable. In some embodiments, these scaffolds possess no enzymatic activity and allow the fast and cost-effective production in bacterial (like E. coli) or in vitro translation systems for personalized cancer immunotherapies. In some embodiments, scaffold domains having high expression in bacterial or yeast or other host cell expression systems are considered. In some embodiments, structurally and biochemically characterized scaffolds and those having no toxicity when used as a vaccine are considered. In some embodiments, the scaffold domains do not require post-translational modification for correcting folding. In some embodiments, the scaffold domains do not require disulfide-bond formation. For generation of a second-generation polybody, in some cases, scaffold domains that would allow engineering functions are considered. In some cases, these domains can be used to select for binders (agonistic and/antagonistic) against molecular targets and be able to function like antibody-mimetics.

1.2. Selecting Scaffolding Domains with the Highest Solubilization Power

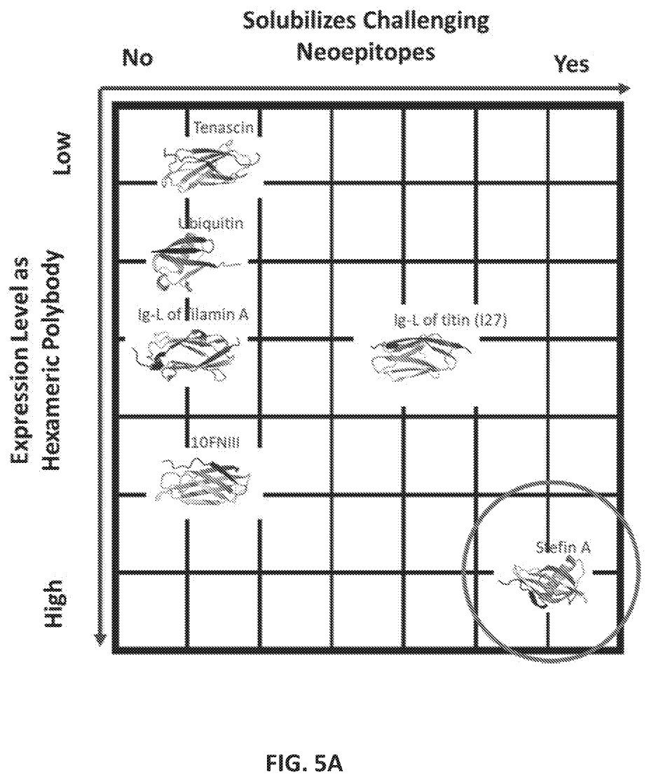

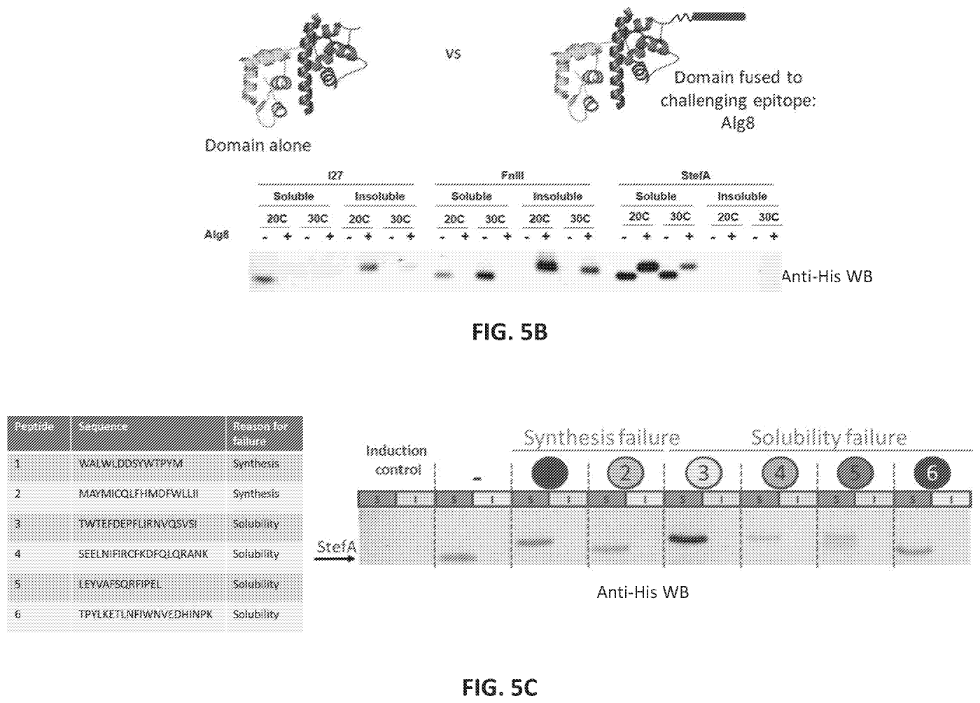

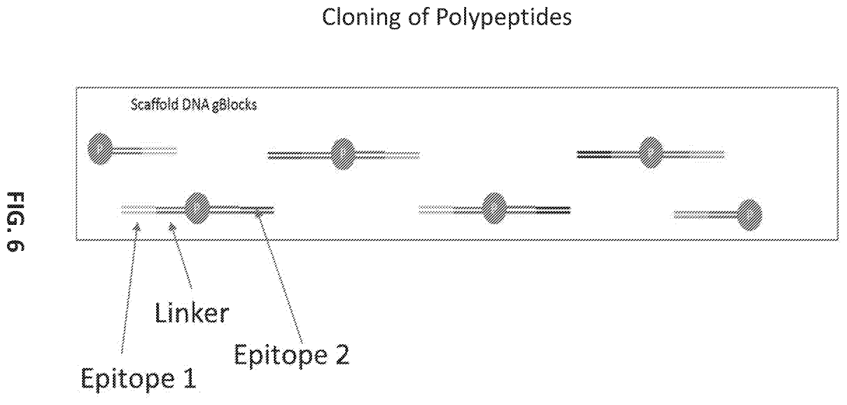

In some embodiment, domains that are able to solubilize and carry a large variety of different epitopes are preferable. To test the efficacy of solubilizing challenging epitopes, stress tests were performed with the Alg8 epitopes from the T3 mouse tumor model that has strong tendency for aggregation and precipitation in aqueous solution. The Alg8 epitopes were C-terminally fused to different monomeric scaffolding domains and tested for their soluble expression in a bacterial expression system. Almost all scaffolding domains expressed well. Under the conditions, Stefin A and titin-127 domains were expressed as a soluble Alg8-fusion protein, and Stefin A had better solubilizing power (FIGS. 5A-5B). Additionally, various peptides were C-terminally fused to a Stefin A scaffolding domain and tested for their soluble expression in the bacterial expression system. The results demonstrated that fusion to Stefin A was capable of solubilizing multiple different peptides that were difficult to synthesize or solubilize alone. FIG. 5C).

### 1.3. Testing Various Polybody Configurations

Polybodies can be generated in multiple configurations that can have distinct characteristic and benefits as a vaccine technology to induce potent anti-tumor immunity. For example, the polybody can have multiple scaffolding domains fused together via linker regions. Constructs with just one, two, four or 6 scaffolds linked to together were generated. The fusion of multiple scaffolds generate molecules with larger molecular weight which might be a desired vaccine function because these molecules can be large enough to traffic into the lymphatic system after subcutaneous injection and therefore accumulate preferentially in the lymph node where they encounter a high density of professional antigen presenting cells. Under certain conditions, titin-127 and Stefin A domains were able to be configured in such a multivalent structure with high efficacy. For another example, epitopes can be inserted between each domain of the polybody or as a string of epitopes between two domains, at C-terminus or N-terminus. In some cases, one of the robust ways to incorporate 5 epitopes into a polybody with 6 scaffolding domains is the insertion of each single epitope between two domains rather than an insertion of a string of 5 epitopes at the ends or middle of the polybody.

In some cases, other factors can affect the ability to generate these polybodies and their immunogenicity as a vaccine technology. For example, the linker regions between each scaffold and epitope can be designed in such a way that they support flexibility as well as solubility. Furthermore, these linker regions can be optimized for efficient cleavage and presentation of the epitope, e.g., by containing cleavage sites for peptidases in the antigen presentation pathway or by adding the sequence context of well-presented epitopes or based on a consensus sequences from spectrometry data from HLA-immunopeptidomes studies (see, e.g., Abelin et al., Immunity, 2017).

### Example 2—Cloning Polybodies

In some embodiments, the design of the polybody structure includes repeating scaffold domains, strung together via linker regions as beads along a string. These domains can contribute to polybody solubility, stability, and/or functionality and can keep the epitopes of interest protected from excessive unwanted degradation. The following approach illustrates an exemplary low throughput cloning of polybodies. A master construct was created, which contained the six scaffold domain genes interspaced by flexible linker regions with unique restriction enzyme sites in the center of the linkers. These restriction enzyme sites enable insertion of neo-epitope DNA sequences using classical restriction-based cloning or Gibson assembly molecular cloning techniques in a stepwise manner.

In some cases, the repetition of identical Stefin A domains can be used to clone the polybody construct in a single reaction needed for high-throughput applications. In some cases, individual Stefin A domains attached with linkers and variable epitope sequences (at 5′ and 3′ end) are synthesizable and can be used to develop a fast cloning approach. For example, as illustrated in FIG. 6, the sequence differences in the epitopes are used for annealing to a complementary region on 5′ end of another DNA piece that encodes a second epitope on its 3′ end which than anneals to the complementary 5′ sequence on the following DNA piece.

### Example 3—Immunological Testing

### 3.1. Lymph Nodes Targeting

One of the major challenges in effective vaccine development is the delivery of antigen to secondary lymphoid organs, where antigen presenting cells can encounter and prime T cells. For example, subcutaneous or intradermal delivery does not always result in effective biodistribution of the delivered components. For vaccines to effectively trigger robust T cell response the antigens may be specifically localized to the lymph nodes, e.g., the axillary and inguinal lymph nodes. One method to achieve lymph node retention is to increase molecule size of the antigens. To have effective lymph node uptake, antigens can be either fused to large carrier molecules or modified so that it can bind to endogenous proteins of enough size. Fusion proteins, nanoparticles or albumin hitchhiking can be used to enhance vaccine lymph node targeting.

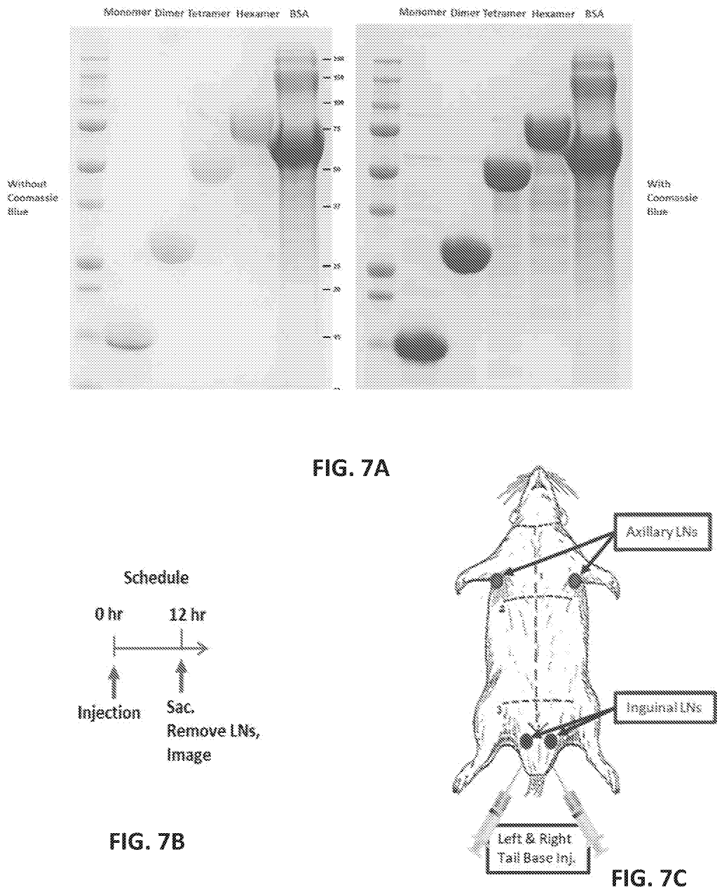

Accordingly, disclosed herein are polybodies with enough size advantage designed to be retained in lymph node (LN), as well as being used to solubilize and stabilize neoepitopes, especially the neoepitopes that are difficult to synthesize or solubilize. To confirm that the polybody was indeed retained in LNs, experiments were designed to compare polybodies vs. synthetic long peptide (SLP) antigens to determine if there is preferential accumulation in the LN (FIG. 7A-FIG. 7C, Table 1).

TABLE 1

| Group | [symbol] | [symbol] | [symbol] | [symbol] | [symbol] | [symbol] | [symbol] |
|---|---|---|---|---|---|---|---|
| 1 | [symbol] | [symbol] | [symbol] | [symbol] | [symbol] | [symbol] | [symbol] |
| 2 | [symbol] | [symbol] | [symbol] | [symbol] | [symbol] | | |
| 3 | [symbol] | [symbol] | [symbol] | [symbol] | [symbol] | | |
| 4 | [symbol] | [symbol] | [symbol] | [symbol] | [symbol] | | |
| 5 | [symbol] | [symbol] | [symbol] | [symbol] | [symbol] | | |
| 6 | [symbol] | [symbol] | [symbol] | [symbol] | [symbol] | | |
| 7 | [symbol] | [symbol] | — | [symbol] | [symbol] | | |

[symbol]

indicates data missing or illegible when filed

Fluorophore labelled antigenic peptide and protein monomer, dimer, tetramer and hexamer were prepared using Albumin from Bovine Serum (BSA) Alexa Fluor™ 647 conjugate from ThermoFisher. Purified Stefin A proteins were labeled with Alexa Fluor™ 647 NHS Ester (Succinimidyl Ester) reactive dye and cleaned with a Zeba desalting column and HPLC was performed (OD at 280 nm and 647 nm).

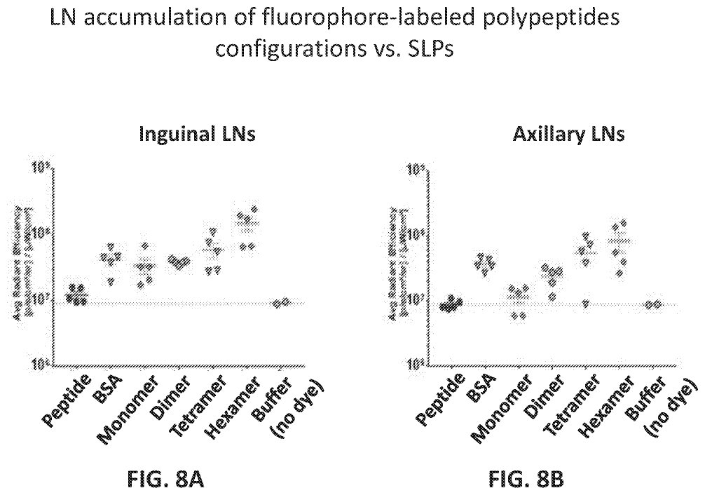

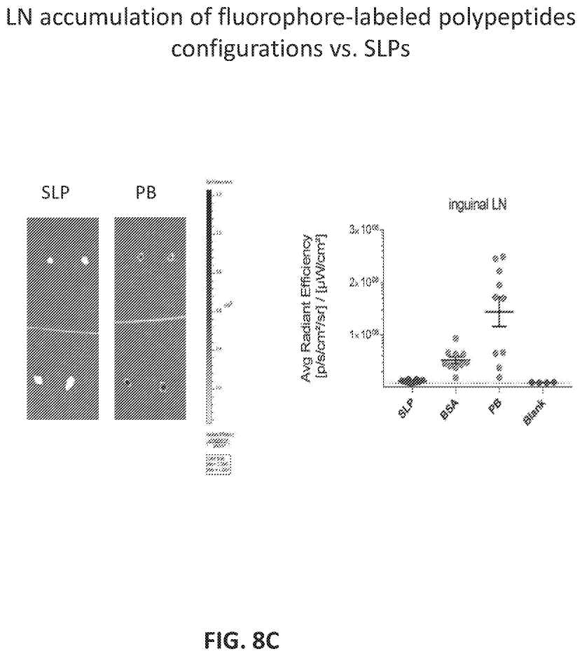

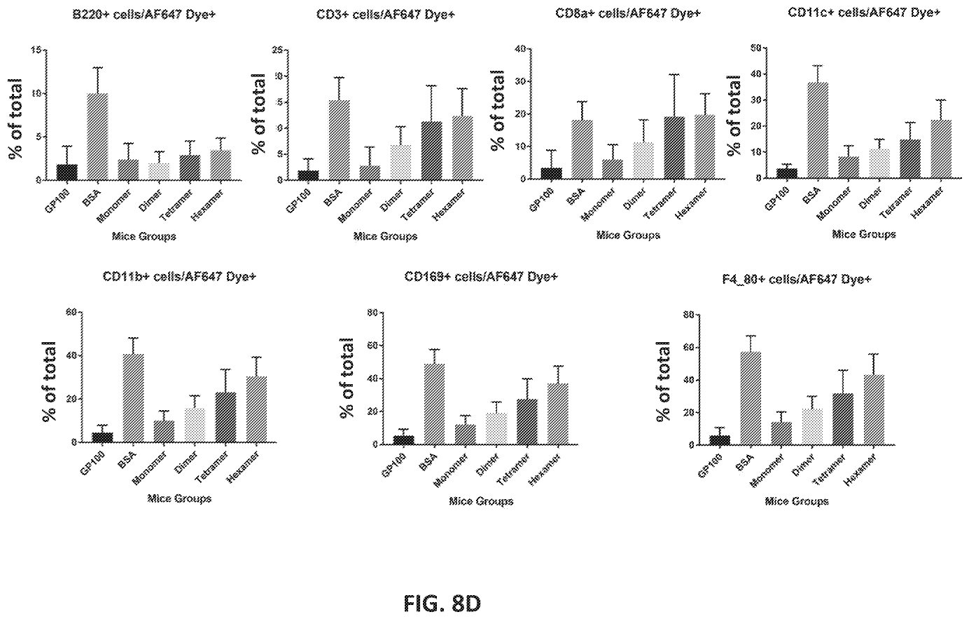

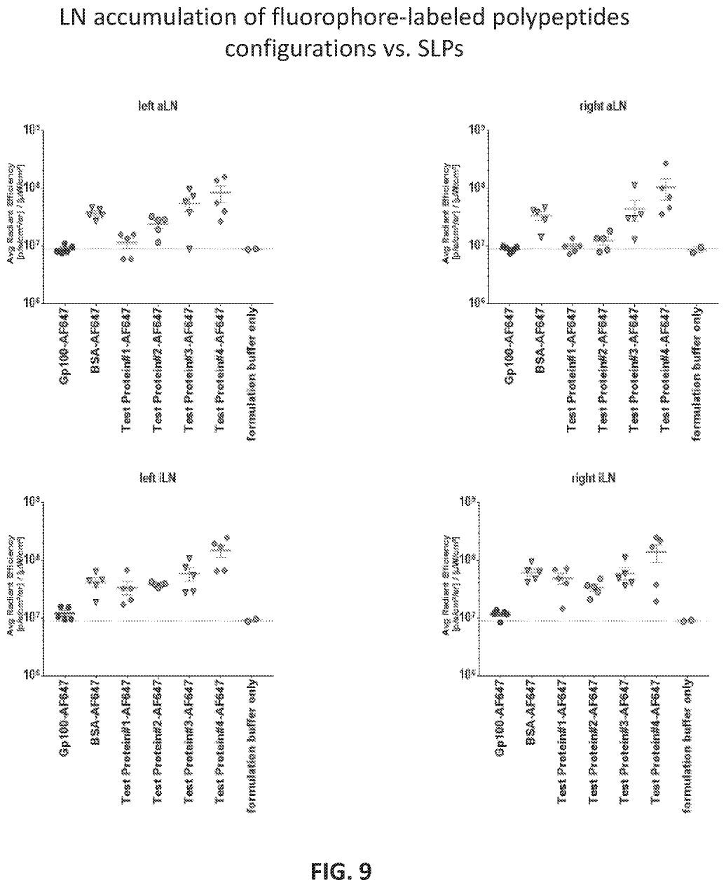

Fluorophore labelled antigenic peptide and protein monomer, dimer, tetramer and hexamer were injected into mice subcutaneously to the left and right tail base, with Alexa647_BSA as a positive control. Twelve (12) hours after dosing, animals were euthanized by CO₂. Left and right axillary and inguinal lymph nodes (aLN-L, aLN-R; iLN-L, iLN-R) were isolated and imaged ex-vivo by Lumina Series III In Vivo Imaging System (IVIS; PerkinElmer). As illustrated in FIG. 8A-FIG. 8C and FIG. 9, there was a positive correlation between number of domains in polybody and the extent of LN accumulation in both inguinal and auxiliary LNs. As the number of domains in a polybody increased, LN accumulation also increased. Hexamer polybody showed LN accumulation that was markedly higher than the positive control BSA, probably due to larger size or its unique chain-like configuration. The molecular weights of the polybodies, the SLP, and the BSA are shown in Table 2 below.

TABLE 2
Molecular weight of Polybodies, SLP, and BSA

| | | Pos. | | | | |
|---|---|---|---|---|---|---|
| | | Control | Polybody Configurations | | | |
| | SLP | BSA | Monomer | Dimer | Tetramer | Hexamer |
| MW (kD) | 3.6 | 68 | 13 | 26 | 52 | 78 |

Lymph nodes were then processed, and cells were resuspended and analyzed with FACS according to Table 3 below. Results of the FACS analysis are shown in FIG. 8D.

TABLE 3

| | | | | | | | Test |
|---|---|---|---|---|---|---|---|
| | | | | | Catalog | | Amount |
| Purpose | Marker | Fluorophore | Clone | Company | # | Laser | (μl) |
| Viability | Live/Dead | APC-Cy7 | N/A | Life | L10119 | Red | 1:1000 in |
| | IR | | | Technologies | | | staining |
| | | | | | | | buffer |
| DCs | CD11c | FITC | HL3 | BD | 553801 | Blue | 2 |
| Dye-label | Polybodies | Alexa-647 | N/A | Neon | N/A | Red | |
| DCs | CD8a | BUV395 | 53.6-7 | BD | 563786 | UV | 2 |
| DCs | CD11b | BV711 | M1/70 | BD | 563168 | Violet | 2 |
| Macrophages | F4/80 | Pacific | BM8 | Biolegend | 123124 | Violet | 2 |
| | | Blue | | | | | |
| T cells | CD3 | PE-Cy7 | 17A2 | BD | 560591 | YG | 2 |
| B-cells | CD45R/ | PerCP- | RA3- | Biolegend | 103236 | Blue | 2 |
| | B220 | Cy5.5 | 6B2 | | | | |
| Macrophages | CD169 | PE | 3D6.112 | Biolegend | 142404 | Blue/ | 2 |
| | (Siglec-1) | | | | | YG | |

### 3.2. Immunogenicity

To compare immunogenicity of neoantigens encoded in constructs comprising polybodies vs synthetic long peptides (SLPs), C57BL/6J mice were injected s.c. into their tail base on both side with 10 μg of SLPs or molar equivalent of polybodies carrying the same epitopes, together with adjuvant. Two rounds of injections for priming and boosting were carried out on day 0 and day 14. The mice were sacrificed on day 21 and their spleens were collected for immune cytokine detection. Blood was also drawn on day 0, 7, 14 and 21 for epitope specific CD8+ cell multimer staining. Peptides used in the study include: GIPVHLELASMTNMELMSSIVHQQVFPT (adpgk) and GRVLELFRAAQLANDVVLQIMELCGATR (Reps1), both of which were from mouse MC38 colon carcinoma tumor model.

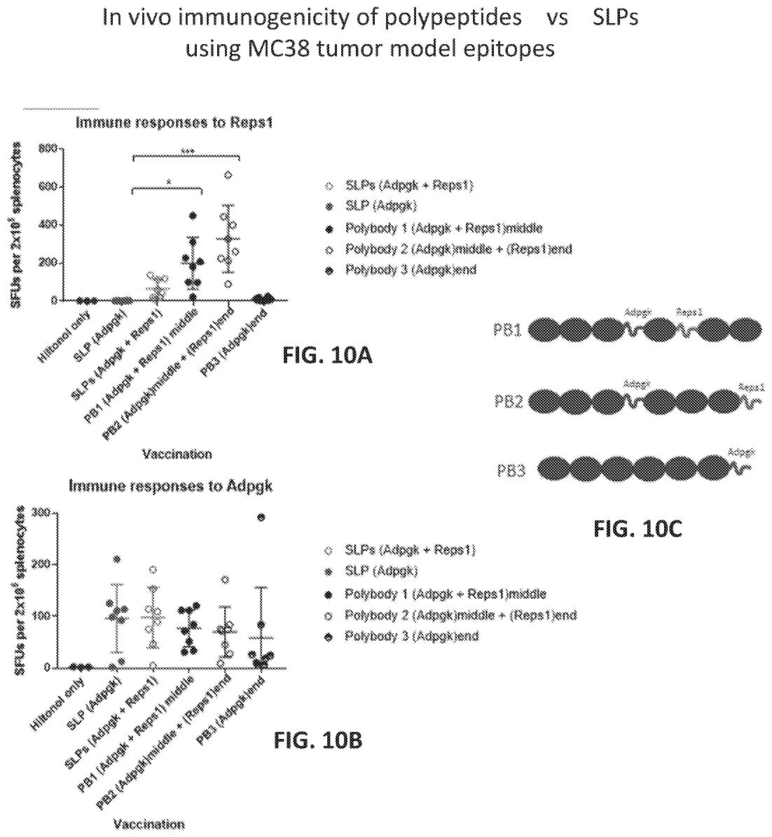

As illustrated in FIG. 10A-FIG. 10C, mice immunized with polybody containing Reps1 showed stronger specific T cell responses comparing with mice immunized with the same SLPs. Vaccination with polybodies containing Reps1 in the middle induced a 2-fold increase in antigen-specific T cell responses as compared to SLPs, while vaccination with polybodies containing Reps1 at the end induces about a 300-fold increase in antigen-specific T cell responses as compared to SLPs. There were also no cross-reactivity observed with adpgk from polybody carrying both epitopes. On the other hand, adpgk-specific T cell responses observed in mice immunized with either SLPs or with polybodies carrying adpgk were similar. Polybodies encoding adpgk alone at the end tend to elicit lower immune responses than the other polybodies or even SLPs, possibly due to end clipping.

### Example 4—Functionalized Polybodies

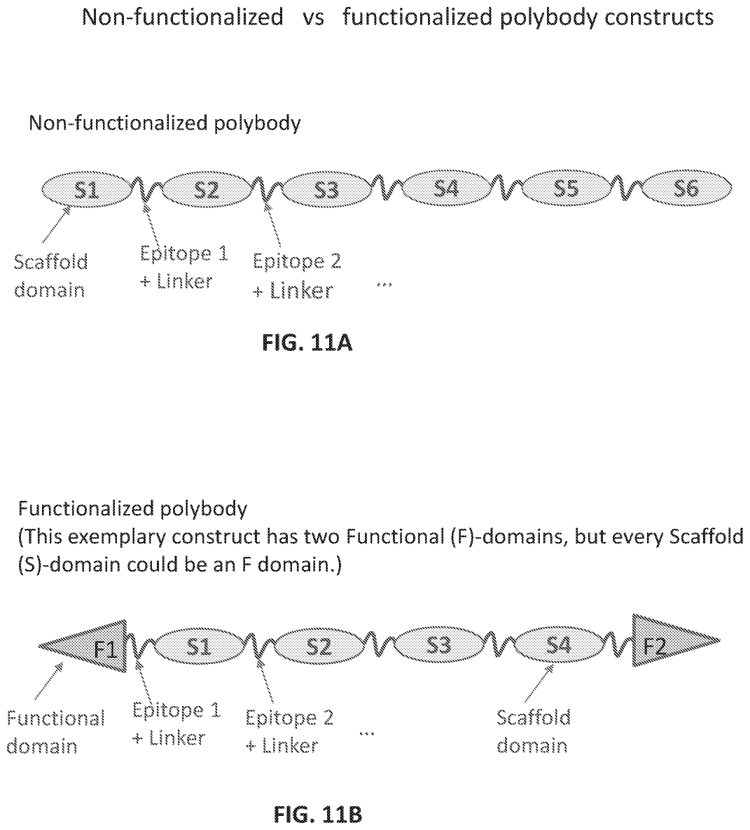

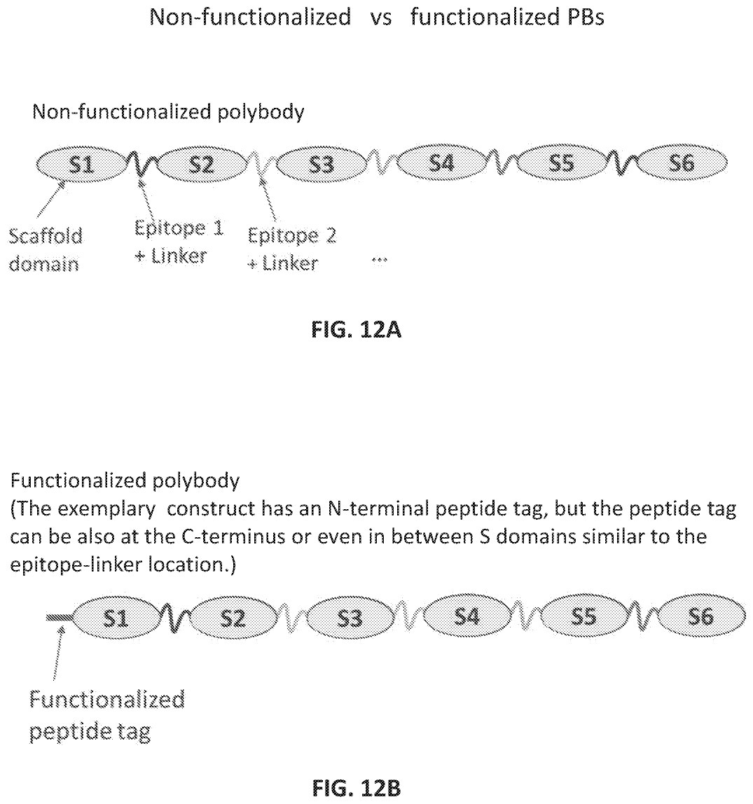

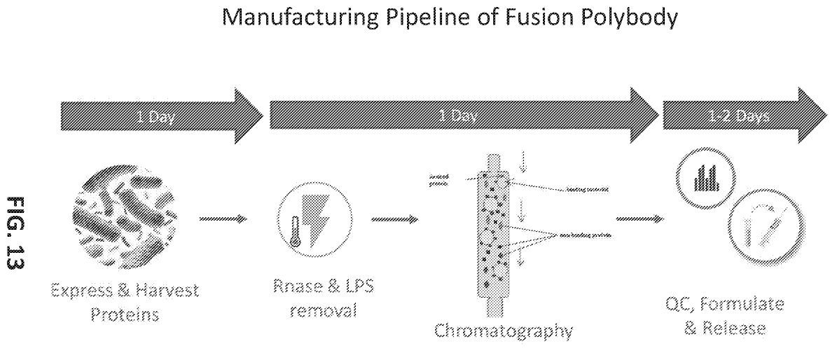

The polybodies can be configured to have functionalities, either individually or in combination with other functions. Polybody constructs comprising epitopes can be generated without additional functional specializations (e.g., FIG. 11A and FIG. 12A). Exemplary functionalized polybody configurations are illustrated in FIG. 11B and FIG. 12B. The functions can include, but are not limited to, dendritic cells targeting, complex formation with adjuvant, and complex formation with reagents that facilitate endosomal escape, cross-presentation, or co-delivery with other drugs. A generalized schematic diagram representing manufacturing pipeline of fusion polybody is demonstrated in FIG. 13.

Dendritic cells (DCs) are professional antigen presenting cells that are critical for capturing, processing and presenting antigens on the cell surface to prime T cells, and then controlling T cell expansion and promoting their effector function. Clec9A is a surface C-type lectin-like molecule that is expressed on the surface of a selective group of DCs. In humans, it is expressed by BDCA3+ myeloid dendritic cells (mDCs), which are essential for antigen cross-presentation. Antigens conjugated to antibodies to Clec9A or Clec9A targeting molecule can cause increased immune response and can enhance antitumor immunity in animal model. Clec9A can be a target for antigen DC targeting to increase the efficiency of cancer vaccines.

The DC-targeting function can be added to the polybodies in multiple ways. For example, one or more of the scaffolding domains can be engineered in such a way that they bind to a DC-receptor like Clec9a. In some embodiments, the DC-targeting peptide sequences can be encoded within the polybody (either at the N- or C-terminus or at some of the normal epitope locations). In some embodiments, DC-targeting peptides can be conjugated to the polybody, e.g., using crosslinkers to target lysine residues on the scaffolding domain. In some embodiments, a DC-targeting peptide sequence can be fused to a peptide sequence that binds the scaffolding domains of the polybody and then this bifunctional peptide can be mixed with the polybody during formulation. In some embodiments, the multivalent nature of the polybody will allow high avidity binding of this DC-targeting peptide via the scaffolding part (e.g., at six sites) and therefore display multiple DC-targeting peptides.

### Example 5—Method of Preparing Enriched Artificial Mini-Proteome Vaccine

In this example a simplified method of generating artificial mini-proteome vaccine (AmpVax) is described. Briefly, total RNA is extracted from FFPE samples. Messenger RNA can be partially fragmented due to FFPE storage or during RNA extraction steps. Using random primer, cDNA strands representing the entire exome, each strand approximately 200 bp long are generated using a commercial kit. Each cDNA strand is ligated to a strand-specific adaptor that selectively allows for expression of the coding strand (FIG. 14 and FIG. 15). The adaptor-ligated, purified cDNA is then hybridized to a reference exome capture probe.

Optionally, mismatched heterodimers are then separated by binding to a MutS protein and passing through a MutS capture column. (FIG. 15, detailed in expanded square section at the bottom). MutS column captures the strands that have a mismatch in the double stranded DNA (depicted in the figure with solid fat arrows), whereas the homoduplexes run through. The MutS column-eluted heterodimers represents polypeptides that are enriched from the cancer exome that comprise at least one point mutation. The enriched cDNAs are cloned in a plasmid for expression.

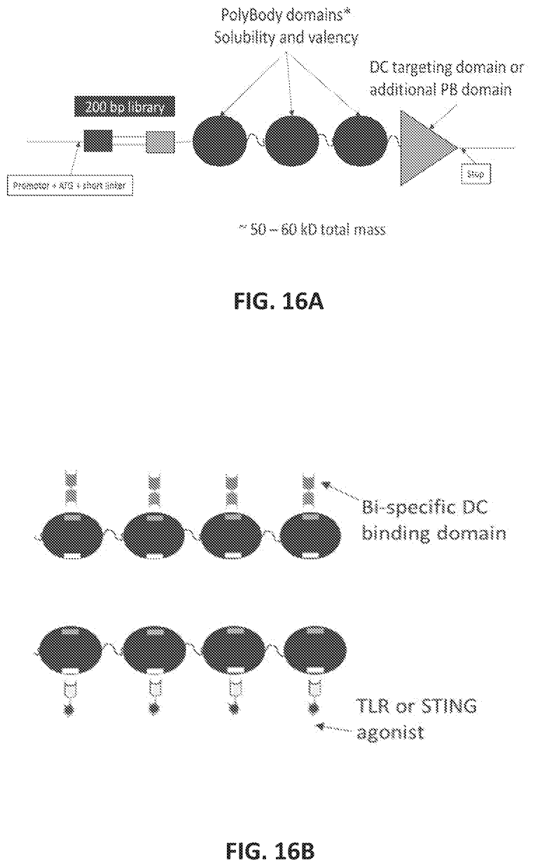

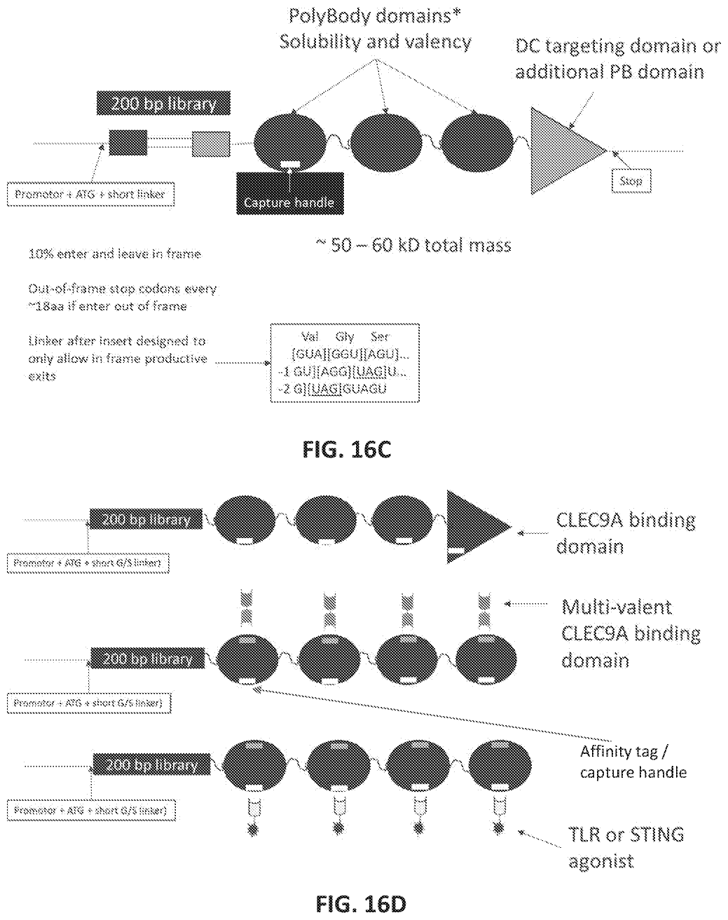

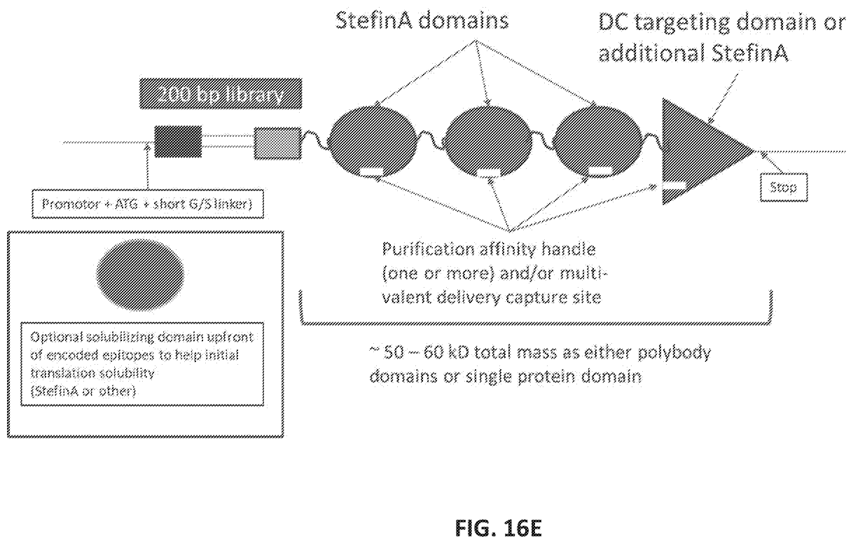

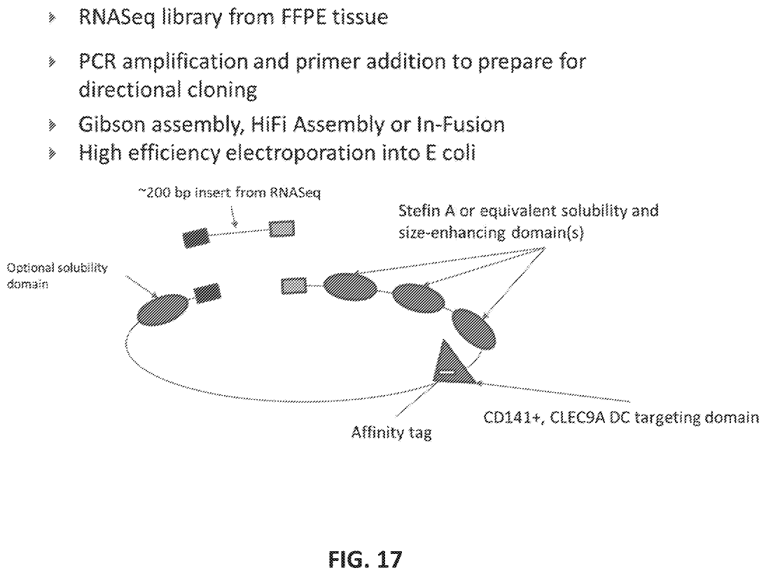

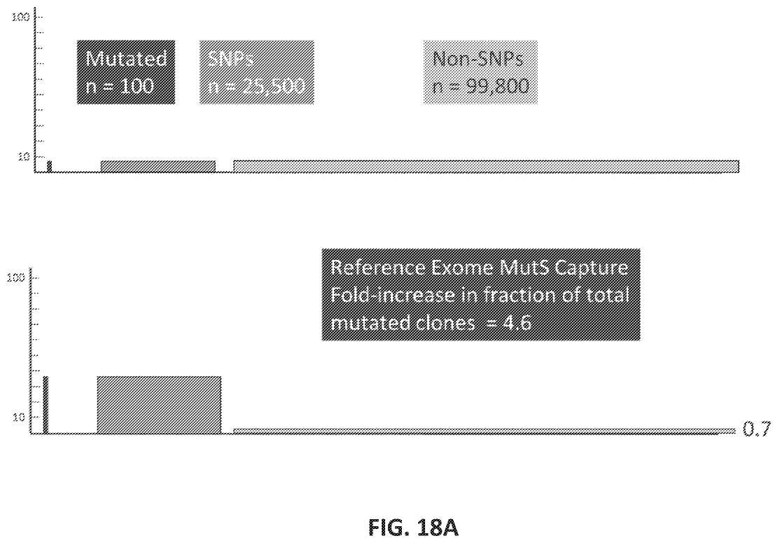

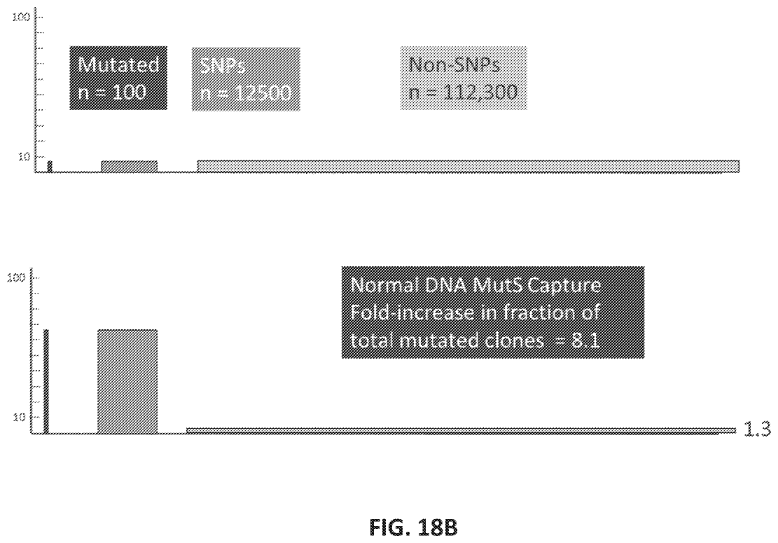

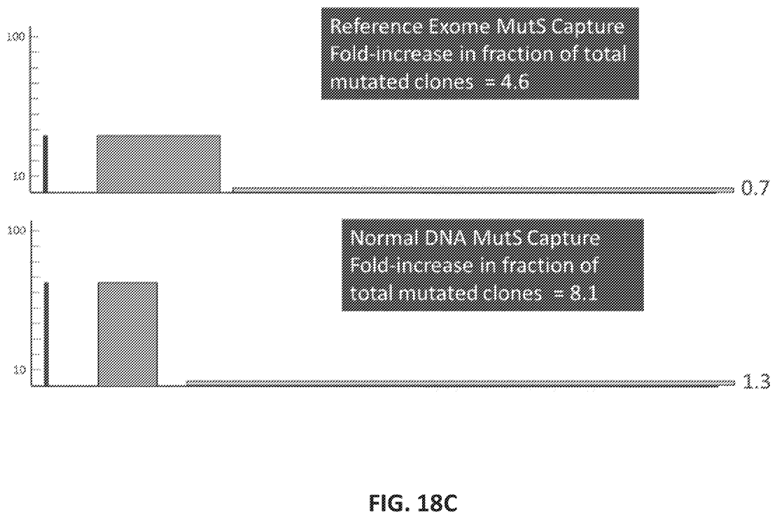

Further modifications to the enriched polypeptide are done by fusion with polynucleotides encoding peptides for generation of polybodies. FIG. 16A and FIG. 16B represent graphically the various modifications that are achieved by creating scaffold proteins that are further fused with DC targeting moieties or with immunogenic proteins such as TLRs, or STING proteins.

1.-4. (canceled)

5. A composition comprising a fusion polypeptide, said fusion polypeptide comprising a polypeptide sequence comprising:
(a) one or more antigen polypeptides sequences, and
(b) one or more scaffold polypeptide sequences,
wherein at least one of the scaffold polypeptide sequences is connected to at least one of the one or more antigen polypeptide sequences through one or more linker sequences, and
wherein the fusion polypeptide is configured to facilitate a cleavage of the linker, a cleavage of at least one of the one or more antigen polypeptides, or presentation of at least one of the one or more antigen polypeptides.

6. The composition of claim 5, wherein the antigen polypeptides are cancer antigens, auto-immune antigens, or viral antigens.

7.-10. (canceled)

11. The composition of claim 5, wherein the scaffold polypeptides are each independently selected from Titin I27, ubiquitin, Stefin A, 10FN-III, Ig-L filamin A, tenascin, fibronectin, a fragment thereof, and a variant thereof.

12.-126. (canceled)

127. A library comprising a plurality of recombinant expression constructs, wherein each expression construct of the plurality comprises:
(a) a promoter sequence;
(b) a sequence encoding a fusion polypeptide comprising:
(i) a start codon downstream of the promoter sequence;
(ii) a first polynucleotide sequence downstream of the start codon, wherein the first polynucleotide sequence comprises a distinct template polynucleotide sequence, wherein the distinct template from polynucleotide sequence (A) is derived from a sample comprising diseased cells from a subject with a disease and (B) encodes a peptide sequence of a protein encoded by the diseased cells from the subject; and
(iii) a second polynucleotide sequence downstream of the first polynucleotide sequence, wherein the second polynucleotide sequence comprises
(A) a frame check sequence and a sequence encoding one or more affinity tags downstream of the frame check sequence, or
(B) a frame check sequence comprising a sequence encoding an affinity tag.

128. The library of claim 127, wherein the frame check sequence operates to terminate translation of the fusion polypeptide when the distinct template polynucleotide sequence or copy thereof is out of frame with the sequence encoding an affinity tag.

129. The library of claim 127, wherein the frame check sequence has a formula of:
N₁-N₂-N₃-N₄-N₅-N₆-N₇-N₈-N₉:
wherein:
each N is independently a nucleic acid selected from the group consisting of A, T, U, C and G;
each of N₁-N₂-N₃ and N₄-N₅-N₆ and N₇-N₈-N₉ is not a stop codon; and
each of N₂-N₃-N₄ and N₆-N₇-Ng is a stop codon.

130. The library of claim 127, wherein the frame check sequence encodes (a) Val-Gly-Ser; and/or (b) a linker that links the first polynucleotide sequence to the sequence encoding the affinity tag.

131.-132. (canceled)

133. The library of claim 127, wherein the affinity tag is (a) a fragment crystallizable region (Fc region) or peptide sequence that binds to an Fc receptor, a Strep-tag, a GST-tag, a His-tag, a peptide sequence that binds to Protein A, a peptide sequence that binds to Protein G or an epitope of an antibody or binding fragment thereof; (b) non-immunogenic; and/or (c) human.

134.-135. (canceled)

136. The library of claim 127, wherein (i) the sequence encoding the affinity tag encodes a size-enhancing polypeptide; and/or (ii) each expression construct of the library comprises a sequence encoding a size enhancing polypeptide.

137. The library of claim 136, wherein the sequence encoding the size enhancing polypeptide is downstream of the first polynucleotide sequence and/or at least one of the sequences encoding the size enhancing polypeptide is upstream of the distinct template polynucleotide sequence.

138. The library of claim 136, wherein the affinity tag is an epitope of a size enhancing polypeptide.

139. The library of claim 136, wherein the sequence encoding the size enhancing polypeptide encodes (a) a plurality of at least 2, 3, 4, 5, 6, 7, 8, 9 or 10 or more size enhancing polypeptides; and/or (b) one or more linkers between two or more of the size enhancing polypeptides.

140.-141. (canceled)

142. The library of claim 139, wherein the molecular weight of the plurality of size enhancing polypeptides is at least 15 kDa, no more than 200 kDa, and/or from 40 kDa to 80 kDa or from 50 kDa to 70 kDa.

143.-145. (canceled)

146. The library of claim 127, wherein the subject is a human.

147. The library of claim 127, wherein the disease is cancer or an autoimmune disease.

148.-152. (canceled)

153. The library of claim 127, wherein each of the distinct template polynucleotides sequences is cDNA, is derived from an RNA molecule, or is derived from genomic DNA (gDNA).

154.-155. (canceled)

156. The library of claim 127, wherein the library comprises a plurality of distinct template polynucleotides that (a) represent at least 5% up to 100% of an exome; (b) encode at least 5% up to 100% of the peptide sequences of a proteome; and/or (c) encode peptide sequences derived from at least 5% of the proteins of a proteome.

157.-158. (canceled)

159. The library of claim 156, wherein the exome or proteome is the exome or proteome of the diseased cells.

160. The library of claim 127, wherein (a) from about 5% to about 25% of polypeptides expressed from the library comprise the affinity tag; (b) from about 20% to about 40% of polypeptides expressed from the library comprise the size-enhancing polypeptide; (c) from about 50% to about 90% of polypeptides expressed from the library that comprise at least 60 amino acid residues which do not comprise the affinity tag; and/or (d) from about 85% to about 95% of the polypeptides expressed from the in frame expression constructs comprise a polypeptide that is attached to the affinity tag.

161.-163. (canceled)

164. The library of claim 127, wherein (a) the library comprises at least 100, 1000, 10000, 100000, 1×10⁶, 1×10⁷, 1×10⁸, 1×10⁹ or up to 1×10¹⁰ distinct template polynucleotide sequences; and/or (b) the distinct template polynucleotides encode peptide sequences derived from at least 10, 50, 100, 500, 1000, 10000 or 100000 distinct proteins.

165. (canceled)

166. The library of claim 127, wherein at least about 10, 100, 500, 1000, 5000 or 10000 of the distinct template polynucleotides encode a mutant peptide sequence or mRNA translation products derived from aberrantly transcribed, spliced or translated mRNA in the diseased cell or from gene rearrangements.

167. The library of claim 166, wherein the mutant peptide sequence is (a) a cancer-cell specific mutant peptide sequence; and/or (b) comprises a point mutation or an indel.

168.-171. (canceled)

172. A polypeptide library encoded by the library of claim 127.

173.-183. (canceled)

184. A personalized recombinant proteome library comprising a plurality of recombinant fusion polypeptides expressed in a host cell, the plurality of recombinant fusion polypeptides comprising a plurality of polypeptide sequences encoded by a plurality of at least 10 distinct template polynucleotide sequences from a sample comprising diseased cells from a subject with a disease, and
wherein each of the polypeptide sequences encoded by the plurality of at least 10 distinct template polynucleotides comprises, in a N to C direction:
(i) a polypeptide sequence encoded by a distinct template polynucleotide of the at least 10 distinct template polynucleotide sequences, and (ii):
(A) a frame check sequence and a sequence encoding an affinity tag downstream of the frame check sequence, or
(B) a frame check sequence comprising a sequence encoding an affinity tag, and/or
(C) a size enhancing polypeptide sequence that is at least 40 kDa.

185.-186. (canceled)

187. A method of treatment comprising administering the polypeptide library of claim 172 to the subject.

188.-223. (canceled)
